(19) United States
(12) Patent Application Publication
(10) Pub. No.: US 2020/0023071 A1
(43) Pub. Date: Jan. 23, 2020

(54) IMMUNOMODULATORY ANTIBODY DRUG CONJUGATES BINDING TO A HUMAN MICA POLYPEPTIDE

(71) Applicant: INNATE PHARMA, MARSEILLE (FR)

(72) Inventors: MATHIEU BLERY, MARSEILLE (FR); DELPHINE BREGEON, MARSEILLE (FR); LAURENT GAUTHIER, MARSEILLE (FR); FLORENCE LHOSPICE, MARSEILLE (FR); LAURENT POUYET, MARSEILLE (FR)

(21) Appl. No.: 16/483,758

(22) PCT Filed: Feb. 5, 2018
(86) PCT No.: PCT/EP2018/052801

§ 371 Date: Aug. 6, 2019

Related U.S. Application Data
(60) Provisional application No. 62/455,019, filed on Feb. 6, 2017.

(51) Int. Cl. A61K 47/68; C07K 16/28; A61P 35/00; A61K 31/551; A61K 38/07

(52) U.S. Cl. CPC A61K 47/6849; C07K 16/2833; A61K 47/6803; A61P 35/00; A61K 2039/505; A61K 38/07; C07K 2317/24; C07K 2317/52; C07K 2317/71; A61K 31/551

(57) ABSTRACT

The present invention provides antigen-binding proteins capable of binding to human MICA polypeptides, conjugated to cytotoxic agents. Said conjugates have increased activity in the treatment of disorders characterized by MICA-expressing cells, particularly tumor cells.

### CROSS-REFERENCE TO RELATED APPLICATIONS

This application claims the benefit of U.S. Provisional Application No. 62/455,019 filed 6 Feb. 2017, which is incorporated herein by reference; including any drawings and sequence listings.

### REFERENCE TO THE SEQUENCE LISTING

The present application is being filed along with a Sequence Listing in electronic format. The Sequence Listing is provided as a file entitled “MICA3_ST25 txt”, created Jan. 26, 2018, which is 78 KB in size. The information in the electronic format of the Sequence Listing is incorporated herein by reference in its entirety.

### FIELD OF THE INVENTION

The present invention provides antigen-binding proteins capable of binding to human MICA polypeptides. The antigen-binding proteins have increased activity in the treatment of disorders characterized by MICA-expressing cells, particularly tumor cells.

### BACKGROUND

The immunoreceptor NKG2D is normally expressed on human T cells (e.g. CD8⁺ T cells, γδ T cells) and NK cells. On pre-activated CD8⁺ cells, NKG2D functions as a synergistic co-stimulator of CD28 and TCR signalling via DAP10 association, whereas in NK cells it functions as a direct activator. Upon ligand engagement, NKG2D therefore conveys directly activating or costimulatory signals via the paired DAP10 adaptor protein, thereby promoting cancer and infectious disease immunity.

Various ligands for human NKG2D (hNKG2D) have been identified and characterized, including the major histocompatibility complex class I-related chain A and B polypeptides (MICA and MICB), the UL16-binding protein (ULBP) family, and the retinoic acid early transcript-1 (RAET1) family. MICA is frequently associated with epithelial tumors, induced by microbial infections, and aberrantly expressed in certain autoimmune disease lesions. The structure of MICA is similar to the protein fold of MHC class I, with an α1α2 platform domain and a membrane-proximal Ig-like α3 domain (Li et al 2001 Nat. Immunol. 2:443). MICA and its close relative MICB, which also serves as a ligand for NKG2D, are both polymorphic and the polymorphism has been shown to affect the affinity for NKG2D (Steinle et al. 2001 Immunogenetics 53:279).

In the mouse, which lacks MHC class I chain (MIC) genes, a family of proteins structurally related to ULBP, the retinoic acid early (RAE-1) molecules function as ligands for NKG2D. RAE-1 expression has been shown to be induced by carcinogens and to stimulate antitumor activities of T cells. Murine NKG2D, however, recognizes human MICA polypeptides (Wiemann (2005) J. Immunol. 175:820-829).

The role MICA in cancer biology has been complicated by the fact that MICA is released as a soluble form from the cell surface of tumor cells (e.g., *019 allele) and on the surface of exosomes (*08 allele) (Ashiru et al (2010) Cancer Res. 70(2):481-489)). Soluble MICA (sMICA) can be detected for example at high levels in sera of patients with gastrointestinal malignancies (Salih et al, 2002 J. Immunol. 169: 4098). The MMPs ADAM10 and ADAM17, as well as the disulfide isomerase Erp5, have been reported to have a role in cleavage and shedding of MICA (Waldhauer (2008) Cancer Research 68 (15) 6368-76; Kaiser et al (2007) Nature 447(7143):482-6; and Salih et al. (2002) J. Immunol 169: 4098-4102).

Reports have emerged that NKG2D on NK cells is downregulated by sMICA (Groh et al. (2002) Nature 419, 734-738; Arreygue-Garcia (2008) BMC 8:16; and Jinushi et al. (2005) J. Hepatol. 43(6)1013-1020), leading to less reactive NK cells. This rationale may have emerged because similar systems have been observed among other protein families such as the Ig-like and the TNF superfamily have been shown to be released as a soluble form and that release of the molecules affects cell-cell interactions by reduction of ligand densities and modulates NK cells bearing the respective receptor (Salih et al. (2002) J. Immunol 169: 4098-4102)).

Several groups have proposed addressing the potential immunosuppression caused by soluble MICA via different approaches. One approach involves antibodies that are intended to bind MICA (sMICA) that do not interfere with the interaction between soluble MICA and NKG2D. (Lu S. et al., (2015) Clin. Cancer Res 21(21):4819-30). Another approach, described in PCT publication no. WO2014/140904, involves antibodies that bind sMICA without binding membrane-bound MICA. Other approaches have been proposed, however without disclosure of antibodies or other therapeutic agents suggesting that these approaches may be difficult. For example, PCT application nos. WO2008/131406 and WO2008/036981 suggest inhibiting the ERp5 enzyme that is involved in proteolytic shedding of MICA within the α3 domain through use of MICA mimetic polypeptides or anti-MICA antibodies that bind the α3 domain of MICA. ERp5 and MIC are proposed to form mixed disulphide complexes on the surface of tumor cells, from which soluble MICA (and MICB) is released after proteolytic cleavage near the cell membrane. However, neither WO2008/131406, WO2008/036981 nor any other groups since their publication have reported any actual antibodies that actually interfered with such proteolytic MICA shedding. Various other α3 domain antibodies have been produced, including the BAM03 antibody available commercially (BamOmab (Tubingen, Germany) and antibodies in PCT application nos. WO2013/117647, WO2013/049527 and WO2015/085210.

More recently, membrane bound MICA has been reported to downmodulate the expression of NKG2D on NK and/or T cells (Von Lilienfeld-Toal et al. (2010) Cancer Immunol. Immunother. Volume 59, Issue 6, pp 829-839). Notably, Wiemann (2005), supra, examined MICA Tg mice and concluded that down-regulation of surface NKG2D on nontransgenic splenocytes was most pronounced after cocultivation with splenocytes from MICA transgenic mice in vitro, and only marginally following treatment with sera from H2Kb-MICA mice, whereas incubation with control cells and sera from non-tgLM, respectively, had no effect and that overall data suggest that reduced surface NKG2D on H2-K-MICA NK cells results in NKG2D dysfunction and that NKG2D downregulation is primarily caused by a persistent exposure to membrane-bound MICA in vivo. WO2013/117647 describes use of antibodies that bind with high affinity to all principal MICA alleles in the human population (and further to MICB) that are able to mediate ADCC towards MICA-expressing tumors, including an extensive set of antibodies, notably antibodies that inhibit the interaction between MICA and NKG2D.

To date many antibodies have been developed to target MICA. However, there remains a need for antibodies capable having improved ability to target MICA in patients having cancer.

### SUMMARY OF THE INVENTION

The invention results, inter alia, from the discovery of anti-MICA antibody-drug conjugates that permit highly-effective targeting and depletion of tumor cells, moreover while avoiding or minimizing increases in circulating sMICA and/or MICA shedding.

By investigating the respective contribution of ADCC and drug conjugate mechanism of action of anti-MICA immunoconjugates in solid tumors, the inventors observed that antibodies with as little as two cytotoxic DNA minor groove binder molecules per antibody were highly efficient in eliminating MICA-expressing solid tumors in immunocompetent mice (patient-derived xenografts). The immunoconjugates, despite a particularly low drug:antibody ratio (DAR) of only 2, displayed maximal efficacy at concentrations well below that at which the ADCC mechanism would be expected to have any significant contribution. Moreover, the antibodies employed in the experiments were modified to lack any effector function.

Furthermore, such anti-MICA immunoconjugates bound the α1α2 platform domain (the α1 and/or α2 domain), and without binding to the membrane-proximal segment within the α3 domain that harbors the proteolytic cleavage site for shedding, and yet even in low doses the immunoconjugates were effective in eliminating tumors in animals whose tumors produced significant circulating (shed) MICA without causing an increase or induction in soluble MICA (sMICA). Use of anti-MICA binding agents at low concentrations, notably below concentrations required to mediate significant ADCC, may avoid stabilization of soluble MICA (e.g., protection from degradation) that may occur at higher concentrations, and can be advantageously combined with certain toxins may provide optimal tumor cell depletion while protecting from sMICA-induced immunosuppression.

MICA, at least in tumor cells, may be underdoing cycling, such that intracellular internalization permits immunoconjugates (e.g. with intracellularly cleavable linkers) to mediate cell death sufficiently rapidly to avoid re-expression of surface MICA and subsequent shedding (into circulation). Advantageously, unlike other MICA binding agents that, when used at concentrations that mediate an anti-tumor effect, will induce or increase shed MICA and/or stabilize shed MICA by protecting it from degradation, the immunoconjugates disclosed herein permit targeting of MICA-bearing cells (e.g. tumor cells) while avoiding sMICA stabilization or increase that could in turn cause NKG2D downmodulation on NK and T cells. Anti-MICA antigen binding proteins, particularly when used at low concentrations or doses, can thus be particularly advantageously used to target MICA-bearing cells while preventing sMICA-mediated immunosuppression and may consequently having an immunomodulatory mechanism of action without requirement of an Fc domain or binding to Fcγ receptors on immune cells. The antibodies therefore need not block ERp5 interactions and/or proteolytic shedding, nor otherwise block the enzymatic cleavage site within the membrane-proximal segment within the α3 domain of MICA that gives rise to shedding from tumor cell; consequently; the antibodies may bind the α1 domain, the α2 domain or the α1α2 domain of MICA (the portion of the MICA protein formed from the α1 domain and α2 domain).

In one embodiment, provided is an antigen binding protein, optionally an antibody or antibody fragment, that binds a human MICA polypeptide, wherein the antigen binding protein is covalently bound to a cytotoxic agent, and wherein the antigen binding protein is capable of causing the death of tumor cells without causing a substantial or detectable increase in and/or stabilization of soluble MICA (e.g. sMICA in circulation).

In one embodiment, provided is an antigen binding protein that binds a human MICA polypeptide, wherein the antigen binding protein is covalently bound to a cytotoxic agent, and wherein the antigen binding protein lacks an Fc domain or comprises an Fc domain of human origin that is modified to reduce binding to human Fcγ receptor.

In one embodiment, provided is an antigen binding protein, optionally an antibody or antibody fragment, that binds a human MICA polypeptide, wherein the antigen binding protein is covalently bound to a cytotoxic agent, wherein the antigen binding protein does not induce MICA shedding when administered to an individual at a dose of less than 1 mg/kg body weight, optionally no more than 0.2 mg/kg body weight. In one embodiment, the antigen binding protein is characterized by the ability to cause the death of MICA-expressing tumor cells when administered at said dose.

In one embodiment, provided is an antigen binding protein, optionally an antibody or antibody fragment, that binds a human MICA polypeptide, wherein the antigen binding protein is covalently bound to a cytotoxic agent, and wherein the antigen binding protein does not stabilize soluble MICA in circulation (or protect sMICA from natural protein degradation processes), as assessed in vitro when incubated with MICA-expressing tumor cells, when the antigen binding protein is present at a concentration corresponding to its EC₅₀ for cytotoxicity towards MICA-expressing tumor cells, optionally HCT116 cells.

In one embodiment, provided is an antigen binding protein of the disclosure, for use in the treatment of cancer, for example a colorectal cancer, renal cell carcinoma, lung cancer (e.g. non-small cell lung carcinoma), melanoma, ovarian cancer, endometrial cancer, pancreatic cancer or head and neck cancer. For example, in one embodiment, provided is an antigen binding protein, optionally an antibody or antibody fragment, that binds a human MICA polypeptide, wherein the antigen binding protein is covalently bound to a cytotoxic agent, for use in the treatment of cancer, wherein the antigen binding protein is capable of being administered (or is administered) to an individual in an amount effective to cause the death of cancer cells without causing a substantial or detectable increase in soluble MICA in circulation. In one embodiment, provided is an antigen binding protein, optionally an antibody or antibody fragment, that binds a human MICA polypeptide and is covalently bound to a cytotoxic agent, for use in the treatment of cancer, wherein the antigen binding protein is administered to an individual in an amount effective to cause the death of cancer cells without causing a substantial or detectable increase in soluble MICA in circulation (e.g. where sMICA is assessed at or within 1 day, 1 week or 1 month from the start of treatment), optionally, wherein the amount effective to cause the death of cancer cells is less than the amount effective to mediate depletion of cancer cells via ADCC.

In one aspect of any embodiment herein, the antigen binding protein is furthermore capable of causing the death of tumor cells without causing a substantial or detectable increase in soluble MICB and/or MICB stabilization in circulation.

In one aspect of any embodiment herein, the antigen binding protein or antibody lacks an Fc domain or comprises an Fc domain or portion thereof having decreased or lack of ability to recruit immune effector cells via an Fc domain).

In one aspect of any embodiment herein, the cytotoxic agent is a DNA minor groove binding agent, optionally a pyrrolobenzodiazepine (e.g. a pyrrolobenzodiazepine dimer).

In one aspect of any embodiment herein, the antigen binding protein has an EC₅₀ for cytotoxicity towards MICA-expressing tumor cells of less than 0.01 μg/ml, optionally less than 0.001 μg/ml, optionally between 0.0005 and 0.01 μg/ml, optionally between 0.001 and 0.01 μg/ml, as assessed in vitro by incubation MICA-expressing tumor cells (optionally HCT116 cells) with the antigen binding protein, optionally further under the conditions described in Example 3.

In one aspect of any embodiment herein, the antigen binding protein binds the α1 and/or α2 domain of MICA.

In one aspect of any embodiment herein, the antigen binding protein comprising a cytotoxic agent covalently bound thereto can also be referred to as an immunoconjugate comprising an antigen binding protein covalently bound to a cytotoxic agent, wherein the antigen binding protein binds a human MICA polypeptide.

In one aspect of any embodiment herein, the amount effective to cause the death of cancer cells without causing a substantial or detectable increase in soluble MICA in circulation (or more generally the amount of the antigen binding protein or immunoconjugate administered) is between 0.01 and 1 mg/kg body weight, optionally between 0.01 and 0.2 mg/kg body weight, or optionally between 0.05 and 1 mg/kg body weight. Optionally, in any embodiment, the antigen binding protein is administered in an amount that provides a concentration (e.g. a trough concentration between successive administrations) in circulation and/or tumor tissue that is less than the concentration effective to mediate depletion of cancer cells via ADCC; optionally in an amount that provides a concentration (e.g. a trough concentration between successive administrations) in circulation and/or tumor tissue that is less than the EC₅₀, optionally the EC₂₀, for induction of ADCC towards MICA-expression tumor cells; or optionally in an amount that provides a concentration (e.g. a trough concentration between successive administrations) in circulation and/or tumor tissue that provides less than 90%, 80%, 50% or 20% saturation of MICA on MICA-expressing tumor cells (and optionally further MICB on MICB-expressing tumor cells).

In one aspect of any embodiment herein, the antigen binding protein or immunoconjugate is administered at least twice, optionally at least 3, 4 or 5 times (e.g. the treatment with antigen binding protein comprises an administration cycle comprising at least 2, 3, 4 or 5 successive administrations of the antigen binding protein).

In one aspect of any embodiment herein, an immunoconugate (an anti-MICA immunoconjugate) comprises an antibody that binds to human MICA (and optionally further to human MICB) conjugated to a cytotoxic agent, wherein the antibody comprises an Fc domain of human origin that is modified to reduce binding to human Fcγ receptors. In one embodiment, the antibody or antigen binding protein has decreased effector function and/or Fcγ receptor binding compared to a wild type human IgG1 antibody. In one embodiment, immunoconjugate comprises an Fc domain lacking effector function or having decreased (e.g. compared to a parental Fc domain; a native human IgG1 Fc domain).

In one embodiment, the antigen binding protein binds the MICA*001, *004, *007 and *008 alleles of the human MICA polypeptide (e.g. as expressed at the surface of a cell). In one embodiment, antigen binding protein further binds the human MICB polypeptide.

In one embodiment, the antigen binding protein competes for binding or that binds the same epitope on MICA as the antibodies comprising the heavy and light chain CDR1, 2 and 3 of any of antibodies disclosed herein. In one embodiment, the antigen binding protein competes for binding to MICA with antibody 19E9, 18E8, 6E4 or 9C10. In one embodiment, the antigen binding protein (e.g. antibody) comprises the heavy and light chain CDR1, 2 and 3 of any of the antibodies disclosed herein, or an amino acid sequence at least 50%, 60%, 70% or 80% identical to such CDR.

In one embodiment, provided is a composition of immunoconjugates according to the disclosure, wherein each immunoconjugate has about 6 or 8 cytotoxic agent molecules (e.g., the composition is characterized by a DAR of from about 5 to about 9, about 5.5 to 8.5, about 6 or about 8), optionally wherein the immunoconjugate has decreased or lacks effector function.

In one embodiment, provided is a composition of immunoconjugates according to the disclosure, wherein each immunoconjugate has about 4 cytotoxic agent molecules (e.g., the composition is characterized by a DAR of from about 3.2. to 4.2, about 3.5 to 4.2, about 3.6 to 4.0, or about 3.8 to 4.0), optionally wherein the immunoconjugate has decreased or lacks effector function. In one embodiment, provided is provided is a composition of anti-MICA immunoconjugates, wherein each immunoconjugate has no more than about 4 cytotoxic agent molecules (e.g., the composition is characterized by a DAR of no more than 4.0), optionally wherein the immunoconjugate has decreased or lacks effector function.

In one embodiment, provided is a composition of anti-MICA immunoconjugates according to the disclosure, wherein each immunoconjugate has about 2 cytotoxic agent molecules (e.g., the composition is characterized by a DAR of from about 1.5. to 2.5, about 1.5 to 2.0, about 1.6 to 2.0, or about 1.8 to 2.0), optionally wherein the immunoconjugate has decreased or lacks effector function. In one embodiment, provided is a composition of anti-MICA immunoconjugates, wherein each immunoconjugate has no more than about 2 cytotoxic agent molecules (e.g., the composition is characterized by a DAR of no more than 2.0), optionally wherein the immunoconjugate has decreased or lacks effector function (e.g., the immunoconjugate.

In one embodiment, each molecule of cytotoxic agent is a DNA minor groove binding agent, optionally a pyrrolobenzodiazepine, optionally a pyrrolobenzodiazepine dimer or trimer. Optionally, the molecules of cytotoxic agent within an anti-MICA immunoconjugate share the same chemical structure.

In one embodiment, an anti-MICA immunoconjugate comprises an antibody that binds to human MICA (and optionally further to human MICB) conjugated to a DNA minor groove binding agent, optionally a pyrrolobenzodiazepine, optionally a pyrrolobenzodiazepine dimer or trimer, wherein the antibody comprises an Fc domain of human origin that is modified to reduce binding to human Fcγ receptors, wherein the antibody is conjugated to no more than 4 molecules of the DNA minor groove binding agent.

In one embodiment, the antibody is conjugated to 4 molecules of the DNA minor groove binding agent. In one embodiment, the antibody is conjugated to no more than 2 molecules of the DNA minor groove binding agent. In one embodiment, the antibody is conjugated to 2 molecules of the DNA minor groove binding agent. In one embodiment, provided is a composition of said anti-MICA immunoconjugates, wherein the composition is characterized by a DAR of from 1.5. to 2.5, 1.5 to 2.0, 1.6 to 2.0, or 1.8 to 2.0. In one embodiment, provided is a composition of said anti-MICA immunoconjugates, wherein the composition is characterized by a DAR of from 3.2. to 4.2, about 3.5 to 4.2, about 3.6 to 4.0, or about 3.8 to 4.0

The studies presented herein were conducted using a drug conjugation methodology that permits precise numbers of cytotoxic agents to be conjugated to an antibody, such that substantially all antibodies in a mixture bear the same number of cytotoxic moieties (e.g., a drug:antibody ratio (DAR) of about 2 or 4), and since only specific pre-defined amino acid residues are conjugated the compositions were free of antibody species having greater than the specified number of cytotoxic agent molecules. The methodology permits antibodies and their mechanism of action be compared without bias from differing distributions of DARs within the different antibody samples. Thus, in one embodiment, a composition of the anti-MICA immunoconjugate comprises a plurality of antibodies that binds to human MICA (and optionally further to human MICB) each conjugated to a cytotoxic agent (e.g. DNA minor groove binding agent), wherein at least 80%, optionally at least 90%, of the antibodies or antibody fragments in said composition are conjugated to 2 molecules of cytotoxic agents (e.g. DNA minor groove binding agents). In another embodiment, the anti-MICA immunoconjugate comprises an antibody that binds to human MICA (and optionally further to human MICB) conjugated to a cytotoxic agent (e.g. DNA minor groove binding agent), wherein at least 80%, optionally at least 90%, of the antibodies or antibody fragments in said composition are conjugated to 4 molecules of cytotoxic agents (e.g. DNA minor groove binding agents).

The antibodies may therefore be advantageously used as immunoconjugates that lack Fcγ-mediated effector activity (e.g., do not mediate ADCC), thereby benefitting from potent direct anti-tumor activity despite the presence of tumor cells that shed MICA and/or despite the presence of soluble MICA, and without potential immune toxicity related to Fc receptor binding of immunoconjugates.

In one aspect, as illustrated herein in the antibodies in the Examples using the anti-MICA antibodies immunoconjugates with stoichiometrically functionalized acceptor glutamines wherein substantially all antibodies in a mixture have a particular DAR (e.g., a DAR of 2 or 4), the antibodies are capable of potently inducing tumor cell death as immunoconjugates despite of lack of any Fc-mediated effector function. The antibodies of the disclosure can comprise an Fc domain (or portion thereof) of human IgG1, IgG2, IgG3 or IgG4 isotype modified to reduce or abolish binding to one or more (or all of) human Fcγ receptors (e.g., CD16A, CD16B, CD32A, CD32b, CD64). Such an antibody will minimize take-up by non-tumoral Fcγ receptor-expressing cells, potentially enhancing the patients' immune function, which may in turn be beneficial both for patients having immune infiltrated tumor and patients having non-immune infiltrated tumors.

In one embodiment, as illustrated by antibodies used in the Examples herein, the antibody retains binding to human FcRn proteins thereby providing a vivo half-life. In one embodiment, the anti-MICA immunoconjugates is characterized by low toxicity, e.g. toward immune cells, B cell, etc.

In one aspect of any embodiment herein, the antibody is capable, upon binding to MICA on the surface of a tumor cell, to undergo intracellular internalization.

In one aspect, an anti-MICA immunoconjugate is represented by Formula I:

Ab-(X-Z)ₘ (Formula I)

wherein,

Ab is an anti-MICA antigen binding protein, optionally an antibody or antibody fragment, e.g., an antigen binding protein, antibody or antibody fragment of the disclosure (e.g., an antigen binding protein or antibody lacking an Fc domain or comprising an Fc domain of human origin that is modified to reduce binding to human Fcγ receptors), optionally an antigen binding protein or antibody that binds the α1 and/or α2 domain of MICA (and optionally further MICB), optionally an antigen binding protein or antibody that competes for binding or that binds the same epitope on MICA as the antibodies comprising the heavy and light chain CDR1, 2 and 3 of any of antibodies disclosed herein, optionally an antibody that competes for binding to MICA with antibody 19E9, 18E8, 9C10 or 6E4;

X is a moiety which connects Ab and Z, e.g., the residue of a linker following covalent linkage to one or both of Ab and Z;

Z is a cytotoxic agent (which is to be delivered to a MICA-expressing cell), optionally a DNA minor groove binding agent, optionally a pyrrolobenzodiazepine (e.g. a pyrrolobenzodiazepine dimer or trimer); and

m ranges from about 1 to about 6, optionally from about 1 to about 4, optionally from about 2 to about 4, optionally wherein m is 2 or 4.

In one embodiment, X comprises a linker (e.g., a peptidyl linker) that is cleaved by an intracellular peptidase or protease enzyme, optionally, a lysosomal or endosomal protease.

In one embodiment, the cytotoxic agent is a DNA minor groove binding agent, optionally a pyrrolobenzodiazepine, optionally a pyrrolobenzodiazepine dimer or trimer.

In one embodiment of any aspect herein, an immunoconjugate comprises a cytotoxic agent covalently bound to an amino acid residue (e.g. a lysine residue, a non-natural amino acid residue, or an acceptor glutamine residue (Q)) within or appended to a Fc region of an antibody (e.g., within a peptide or enzymatic recognition tag fused to the C-terminus of a heavy and/or light chain). In one embodiment, the amino acid residue within a Fc region is at a residue in the CH2 and/or CH3 domain, optionally at Kabat position 295 and/or 297 (Kabat EU numbering) of a heavy chain. Optionally, the glycan naturally present at residue N297 is absent or modified (e.g. truncated), wherein the antibody has decreased binding to human Fcγ receptors.

In one aspect, provided is an anti-MICA antibody or antibody fragment of the disclosure (e.g., an antibody lacking an Fc domain or comprising an Fc domain of human origin that is modified to reduce binding to human Fcγ receptors), wherein the antibody comprises a functionalized amino acid residue (B) comprising the structure:

(B)-L″-Y-Z

or a pharmaceutically acceptable salt or solvate thereof,

wherein:

B is an amino acid residue present within or appended to a constant region of the antibody or antibody fragment;

L″ is a linker covalently bonded to the amino acid residue B;

Y is a spacer system; and

Z comprises a cytotoxic agent, optionally a DNA minor groove binding agent, optionally a pyrrolobenzodiazepine.

In one aspect, provided is an anti-MICA antibody or antibody fragment of the disclosure (e.g., an antibody lacking an Fc domain or comprising an Fc domain of human origin that is modified to reduce binding to human Fcγ receptors), wherein the antibody comprises a functionalized amino acid residue (B) comprising the structure comprising the structure:

(B)-L″-RR′—Y-Z

or a pharmaceutically acceptable salt or solvate thereof,

wherein:
B is an amino acid residue present within or appended to a constant region of the antibody or antibody fragment;
L″ is a linker covalently bonded to the amino acid residue B;
(RR′) is an addition product between a reactive moiety R and a complementary reactive moiety R′;
Y is a spacer system; and
Z comprises a cytotoxic agent, optionally a DNA minor groove binding agent, optionally a pyrrolobenzodiazepine.

In one aspect, provided is an anti-MICA antibody or antibody fragment of the disclosure (e.g., an antibody lacking an Fc domain or comprising an Fc domain of human origin that is modified to reduce binding to human Fcγ receptors), wherein the antibody (or antibody fragment) comprises a functionalized acceptor glutamine residue (Q) comprising the structure:

(Q)-L″-Y-Z

or a pharmaceutically acceptable salt or solvate thereof,

wherein:

Q is a glutamine residue present within or appended to a constant region of the antibody or antibody fragment;

L″ is a lysine-based linker in which the nitrogen atom is covalently bonded to the γ carbon of Q as a secondary amine;

Y is a spacer system; and

Z is a cytotoxic agent, optionally a DNA minor groove binding agent.

In one aspect, provided is a composition comprising a plurality of anti-MICA antibodies or antibody fragments (e.g. according to any aspect of the disclosure), wherein at least 90% of the antibodies or antibody fragments in said composition have (m) functionalized acceptor glutamine residues (Q) per antibody or fragment, wherein m is an integer selected from 2 or 4, wherein each of the functionalized acceptor glutamine residues has the structure:

(Q)-L″-Y-Z

or a pharmaceutically acceptable salt or solvate thereof,

wherein:

Q is a glutamine residue present within or appended to a constant region of the antibody or antibody fragment;

L″ is a lysine-based linker in which the nitrogen atom is covalently bonded to the γ carbon of Q as a secondary amine;

Y is a spacer system; and

Z is a cytotoxic agent, optionally a DNA minor groove binding agent.

In one aspect, provided is an anti-MICA antibody or antibody (e.g. according to any aspect of the disclosure), wherein the antibody (or antibody fragment) comprises a functionalized acceptor glutamine residue (Q) comprising the structure:

(Q)-L″-RR′—Y-Z

or a pharmaceutically acceptable salt or solvate thereof,

wherein:

Q is a glutamine residue present within or appended to a constant region of the antibody or antibody fragment;

L″ is a lysine-based linker in which the nitrogen atom is covalently bonded to the γ carbon of Q as a secondary amine;

(RR′) is an addition product between a reactive moiety R and a complementary reactive moiety R′;

Y is a spacer system; and

Z is a cytotoxic agent, optionally a DNA minor groove binding agent, optionally a pyrrolobenzodiazepine (e.g., a pyrrolobenzodiazepine multimer, a pyrrolobenzodiazepine dimer, a pyrrolobenzodiazepine trimer).

In one aspect, provided is a composition comprising a plurality of anti-MICA antibodies or antibody fragments (e.g. according to any aspect of the disclosure), wherein at least 90% of the antibodies or antibody fragments in said composition have (m) functionalized acceptor glutamine residues (Q) per antibody or fragment, wherein m is an integer selected from 2 or 4, wherein each of the functionalized acceptor glutamine residues has the structure:

(Q)-L″-RR′—Y-Z

or a pharmaceutically acceptable salt or solvate thereof,

wherein:

Q is a glutamine residue present within or appended to a constant region of the antibody or antibody fragment;

L″ is a lysine-based linker in which the nitrogen atom is covalently bonded to the γ carbon of Q as a secondary amine;

(RR′) is an addition product between a reactive moiety R and a complementary reactive moiety R′;

Y is a spacer system; and

Z is a cytotoxic agent, optionally a DNA minor groove binding agent, optionally a pyrrolobenzodiazepine (e.g., a pyrrolobenzodiazepine multimer, a pyrrolobenzodiazepine dimer, a pyrrolobenzodiazepine trimer).

In one embodiment of the above structures, the antibodies or antibody fragments binds the α1 and/or α2 domain of MICA (and optionally further MICB).

In one embodiment of the above structures, the antibodies or antibody fragments bind the same epitope as an antibody comprising the respective VH and VL of 19E9, 18E8, 2C10 or 6E4.

In one aspect, an antibody or antibody fragment competes for binding to MICA with any of 19E9, 18E8, 2C10 or 6E4, and/or binds to the same epitope on MICA as any of 19E9, 18E8, 2C10 or 6E4.

Advantageously, the epitope bound by the antibody is present on human MICA polypeptide as expressed on the cell surface, optionally as expressed on the cell surface of cancer cells, e.g. HCT116 cells.

In one aspect, an anti-MICA antigen binding protein, antibody or antibody fragment may be characterized as binding to the predominant MICA alleles from each of two major MICA groups that are determined to represent the main families of MICA: Group 1 alleles that bind NKG2D strongly (including MICA*001, *002, *007, *012, *017 and *018) and Group 2 that bind NKG2D weakly (MICA*004, *006, *008, *009 and *019). By binding to an epitope present on the subset MICA *001, *004, *007 and *008 or *001, *004, *007, *008 and *019, the alleles of both groups that are present in almost all individuals are covered.

In one embodiment, an antigen binding protein, antibody or antibody fragment that is capable of binding MICA alleles has an EC₅₀ for binding to a human MICA*001 that differs by less than 2-log, optionally less than 1-log from its binding affinity for human MICA*004, *007 and/or *008, as determined by flow cytometry for binding to cells expressing at their surface the respective MICA polypeptide cells transfected with one of the respective MICA alleles but that do not express the other MICA alleles). In one embodiment, the antibody has an EC₅₀ for binding to human MICA*004, polypeptide that differs by no more than 1 log, 0.5 log, 0.3 log or 0.2 log, as determined by flow cytometry for binding to cells expressing at their surface human *007 and/or *008.

In one embodiment, an antibody, optionally a tetrameric antibody comprises two Ig heavy chains and two Ig light chains. Preferably the antibody has binding affinity (K_{D}), optionally wherein binding affinity is monovalent, optionally wherein binding affinity is bivalent, for a human MICA polypeptide, optionally to one or all of MICA *001, *004, *007 and *008 polypeptides, of no more than 10⁻⁸ M, preferably less than 10⁻⁹ M, preferably less than 10⁻¹⁰ M, or preferably less than 10⁻¹¹M, as determined by, e.g., surface plasmon resonance (SPR) screening (such as by analysis with a BIAcore™ SPR analytical device).

In one embodiment, an antigen binding protein, antibody or antibody fragment that is capable of binding MICA alleles will bind to epitopes on the MICA protein within the α1 and/or α2 domains that are optimal binding regions for activity yet still are found across principal MICA alleles. The epitopes can optionally be on the lateral side of the α1 and/or α2 domains (e.g. on the non-NKG2D binding face), or optionally on the NKG2D binding face.

In one embodiment, an antigen binding protein, antibody or antibody fragment that is capable of binding MICA comprises a human Fc domain of an isotype that naturally is bound by human FcγR (e.g., FcγRIIIA) but comprising one or more amino acid modifications that decreases (e.g. reduces or substantially abolishes) binding to the human FcγR polypeptides CD16A, CD16B, CD32A, CD32B and CD64.

In one embodiment, an antigen binding protein, antibody or antibody fragment that is capable of binding MICA comprises a human Fc domain of an isotype that naturally is bound by human FcγR (e.g., FcγRIIIA) but which Fc domain lacks or has modified or truncated N297-linked glycosylation (Kabat EU numbering) that decreases (e.g. reduces or substantially abolishes) binding to the human FcγR polypeptides CD16A, CD16B, CD32A, CD32B and CD64.

In one aspect of any embodiment herein, an antibody that binds human MICA comprises an Fc domain that is modified (compared to a wild-type Fc domain of the same isotype) to reduce binding between the Fc domain and human CD16A, CD16B, CD32A, CD32B and/or CD64 polypeptides, wherein the Fc domain comprises an amino acid substitution (e.g. compared to a reference Fc domain, e.g. a human IgG1 Fc domain) in a heavy chain constant region at Kabat positions 234, 235 and 331, optionally at Kabat positions 234, 235, 237 and 331, or optionally at Kabat positions 234, 235, 237, 330 and 331. In one embodiment, the antibody comprises an amino acid substitution in a heavy chain constant region at any three, four, five or more of residues selected from the group consisting of: 234, 235, 237, 322, 330 and 331 (Kabat numbering). In one embodiment, the antibody comprises an amino acid substitution in a heavy chain constant region at any one two or three of residues 234, 235 and 237, and an amino acid substitution in a heavy chain constant region at any one two or three of residues 322, 330 and 331 (Kabat numbering); optionally the antibody further comprises an amino acid substitution in a heavy chain constant region that results in reduced or abolished glycosylation at Kabat residue N297, optionally the antibody comprises a substitution at residue N297, optionally to glutamine optionally to a residue other than glutamine or asparagine. Optionally, a phenylalanine or an alanine is present at Kabat position 234. Optionally, a glutamic acid is present at position 235. Optionally, an alanine is present at position 237. Optionally, a serine is present at position 330. Optionally, a serine is present at position 331. In one aspect of any embodiment herein, an antibody that binds human MICA comprises a human IgG1 Fc domain comprising L234A/L235E/N297X/P331S substitutions, L234F/L235E/N297X/P331S substitutions, L234A/L235E/G237A/N297X/P331S substitutions, or L234A/L235E/G237A/N297X/A330S/P331S substitutions, wherein X is any amino acid other than an asparagine.

In one embodiment, provided is a method of producing and/or testing a MICA-binding agent (e.g., an antibody; an immunoconjugate comprising an antigen binding protein that binds MICA conjugated to a cytotoxic agent), said method comprising: (i) preparing a composition of MICA-binding agents (ii) assessing whether the composition is effective in mediating the depletion of MICA-expressing target cells (e.g. tumor cells), and (iii) further whether the composition induces (a) MICA stabilization (e.g., protection from protein degradation) and/or (b) an increase in soluble MICA when brought into contact with MICA-expressing target cells (e.g. tumor cells). The method may optionally further comprise selecting a MICA-binding agent that is effective in directly depleting MICA-expressing target cells and does not induce MICA shedding and/or cause an increase in soluble MICA when brought into contact with MICA-expressing target cells. Step (ii) may optionally comprise bringing composition of MICA-binding agent into contact with MICA-expressing target cells, optionally in the absence of immune effector cells (e.g., T cells, NK cells). In one embodiment, the antibody lacks an Fc domain or comprises an Fc domain lacking effector function.

In one embodiment, provided is a method of producing and/or testing an anti-MICA immunoconjugate, said method comprising: (i) preparing a composition of anti-MICA immunoconjugates comprising an antigen binding protein that binds MICA conjugated to a cytotoxic agent, and wherein the composition is characterized by a DAR of 1.5. to 2.5, 1.5 to 2.0, 1.6 to 2.0, or 1.8 to 2.0, or optionally wherein the composition is characterized by a DAR of 3.2. to 4.2, about 3.5 to 4.2, about 3.6 to 4.0, or about 3.8 to 4.0, and (ii) assessing whether the composition is effective in directly depleting MICA-expressing target cells (e.g. tumor cells), optionally further whether the composition induces sMICA stabilization and/or an increase in soluble MICA. Step (i) may optionally comprise any of the methods of producing immunoconjugates described herein. Step (ii) may optionally comprise bringing composition of anti-MICA immunoconjugates into contact with MICA-expressing target cells, optionally in the absence of immune effector cells (e.g., T cells, NK cells). In one embodiment, the antibody lacks an Fc domain or comprises an Fc domain lacking effector function.

In another embodiment, provided is a method of producing an antibody that binds a MICA polypeptide comprising culturing a recombinant host cell expressing an anti-MICA antibody described herein, recovering anti-MICA antibody produced by said host cell, conjugating the antibody to a cytotoxic agent (e.g., a DNA minor groove binding agent, a pyrrolobenzodiazepine, etc.), and optionally further comprising formulating said antibody (e.g., with a pharmaceutical excipient) for administration to a human subject. Optionally, the method comprises conjugating the antibody to 2 molecules of cytotoxic agent. Optionally, the composition obtained is characterized by a DAR of 1.5. to 2.5, 1.5 to 2.0, 1.6 to 2.0, or 1.8 to 2.0. Optionally, the method comprises conjugating the antibody to 4 molecules of cytotoxic agent. Optionally, the composition obtained is characterized by a DAR of 3.2. to 4.2, about 3.5 to 4.2, about 3.6 to 4.0, or about 3.8 to 4.0. Optionally, the method further comprises assessing the DAR, and if the DAR corresponds to a pre-determined specification (e.g. a DAR or DAR range as disclosed herein, a DAR of about 2 or 4, etc.), further processing the composition for use as a medicament, optionally formulating said antibody (e.g., with a pharmaceutical excipient) for administration to a human subject.

In another embodiment, provided is a method of producing an antibody that binds a MICA polypeptide, comprising the steps of:

a) providing an antibody or antibody fragment comprising an acceptor glutamine residue;

b) reacting said antibody with a linking reagent comprising a primary amine and a moiety of interest (Z), wherein Z is a cytotoxic agent, optionally a DNA minor groove binding agent, optionally a water-soluble pyrrolobenzodiazepine, in the presence of a TGase, under conditions sufficient to obtain an antibody comprising an acceptor glutamine linked to said moiety of interest (Z), via the linking reagent, optionally wherein the reaction mixture is free of organic solvent or contains less than 10% (v/v), optionally further less than 5%, 4%, 3% or 2% (v/v) organic solvent. In one embodiment, the antibody or antibody fragment comprises two acceptor glutamines (e.g. one acceptor glutamine on each heavy chain or on each light chain), wherein each of the two acceptor glutamines is linked to said moiety of interest (Z). In one embodiment, the antibody or antibody fragment comprises four acceptor glutamines (e.g. two acceptor glutamines on each heavy chain or on each light chain), wherein each of the two acceptor glutamines is linked to said moiety of interest (Z).

In another embodiment, provided is a method of producing an antibody that binds a MICA polypeptide, comprising the steps of:

a) providing an antibody or antibody fragment comprising an acceptor glutamine, wherein the antibody or antibody fragment lacks an Fc domain or comprises an Fc domain characterized by reduced or abolished binding to one or more human Fcγ receptors;

b) reacting said antibody with a linking reagent comprising a primary amine and a moiety of interest (Z), wherein Z is a cytotoxic agent, optionally a DNA minor groove binding agent, optionally a water-soluble pyrrolobenzodiazepine, in the presence of a TGase, under conditions sufficient to obtain an antibody comprising an acceptor glutamine linked to said moiety of interest (Z), via the linking reagent, optionally wherein the reaction mixture is free of organic solvent or contains less than 10% (v/v), optionally further less than 5%, 4%, 3% or 2% (v/v) organic solvent. In one embodiment, the antibody or antibody fragment comprises two acceptor glutamines (e.g. one acceptor glutamine on each heavy chain or on each light chain), wherein each of the two acceptor glutamines is linked to said moiety of interest (Z). In one embodiment, the antibody or antibody fragment comprises four acceptor glutamines (e.g. two acceptor glutamines on each heavy chain or on each light chain), wherein each of the two acceptor glutamines is linked to said moiety of interest (Z).

In another embodiment, provided is a method of producing an antibody that binds a MICA polypeptide, comprising the steps of:

a) providing an antibody having at least one acceptor glutamine residue; and

b) reacting said antibody with a linker comprising a primary amine and a reactive group (R), in the presence of a TGase, under conditions sufficient to obtain an antibody comprising an acceptor glutamine linked to a reactive group (R) via said linker, wherein the reaction mixture is free of organic solvent or contains less than 10% (v/v) or contains less than 5%, 4%, 3% or 2% (v/v) organic solvent; and

(c) reacting, in the presence of organic solvent, optionally in the presence of at least 2%, 3%, 4%, 5% or 10% (v/v) organic solvent:
(i) an antibody of step (b) comprising an acceptor glutamine linked to a reactive group (R) via a linker comprising a primary amine, with
(ii) a compound comprising (a) a moiety (Z), wherein Z is a cytotoxic agent, optionally a DNA minor groove binding agent, optionally a highly hydrophobic or non-water-soluble pyrrolobenzodiazepine, and (b) a reactive group (R′) capable of reacting with reactive group R,
under conditions sufficient to obtain an antibody comprising an acceptor glutamine linked to moiety (Z) via a linker comprising a primary amine. In one embodiment, the antibody or antibody fragment comprises two acceptor glutamines (e.g. one acceptor glutamine on each heavy chain or on each light chain), wherein each of the two acceptor glutamines is linked to said moiety (Z). In one embodiment, the antibody or antibody fragment comprises four acceptor glutamines (e.g. two acceptor glutamines on each heavy chain or on each light chain), wherein each of the two acceptor glutamines is linked to said moiety (Z).

In one embodiment of any aspect, Z is a pyrrolobenzodiazepine dimer or trimer, e.g. a C8/C8′-linked pyrrolobenzodiazepine dimer or trimer.

In one embodiment of any aspect, the acceptor glutamine is positioned within the Fc domain, optionally within the CH2 or CH3 domain of each heavy chain. In one embodiment of any aspect, the antibody comprises one or two acceptor glutamines within the Fc domain, optionally within the CH2 or CH3 domain of each heavy chain. In one embodiment of any aspect, the antibody comprises an acceptor glutamine at Kabat position 295 and/or 297 of each heavy chain. In one embodiment of any aspect, the antibody comprises a peptide tag comprising an acceptor glutamine positioned within or appended to the Fc domain, optionally within the CH2 domain. In one embodiment of any aspect, the antibody comprises a peptide tag comprising an acceptor glutamine fused to the C-terminus of each heavy chain and/or to the C-terminus of each light chain.

In one embodiment, the antibodies are capable of directly inducing (e.g., in the absence of immune effector cells) at least 20%, 30%, 50% or 60% cell death, e.g., in an in vitro assay, of MICA-expressing cells. In one embodiment, the antibodies have an EC₅₀ for cytotoxicity towards MICA-expressing tumor cells of less than 0.01 μg/ml, optionally less than 0.001 μg/ml, optionally between 0.0005 and 0.01 μg/ml, optionally between 0.001 and 0.01 μg/ml, as assessed in vitro by incubation MICA-expressing tumor cells (optionally HCT116 cells) with the antibodies, optionally further under the conditions described in Example 3.

In one aspect, provided is a method of treatment using a depleting antibody that specifically binds MICA (and optionally further MICB) as disclosed herein. The antibodies can be used as prophylactic or therapeutic treatment; in any of the embodiments herein, a therapeutically effective amount of the antibody can be interchanged with a prophylactically effective amount of an antibody.

In one aspect, provided is a method of treating an individual having a cancer with an immunoconjugate of the disclosure, wherein the individual has a solid tumor, optionally a solid tumor characterized by an immunosuppressive or non-inflammatory phenotype, optionally a tumor (or tumor environment) characterized no or low numbers of tumor infiltrating lymphocytes, optionally a tumor (or tumor environment) characterized significant or elevated numbers of myeloid derived suppressor cells (MDSC). Optionally, treating the individual with an immunoconjugate of the disclosure reduces the immunosuppressive or non-inflammatory phenotype or the tumor or tumor environment. Optionally, treating the individual with an immunoconjugate of the disclosure enhances the activity or number of NKG2D-expressing tumor infiltrating lymphocytes. Optionally, treating the individual with an immunoconjugate of the disclosure enhances the activity of an immunomodulatory agent, optionally an agent that mediates ADCC or an agent that neutralizes the activity of PD-1. Optionally, the individual is treated with the immunoconjugate of the disclosure in combination with an immunomodulatory agent, optionally an agent that mediates ADCC or an agent that neutralizes the activity of PD-1.

In one aspect, provided is a method of treating an individual having a cancer with an immunoconjugate of the disclosure, wherein the individual has a solid tumor characterized by MICA and/or MICB expressing cells, e.g. malignant cells or immunosuppressive cells such as MDSC. In one embodiment, the MICA and/or MICB expressing cells are in tumor tissue and/or tumor-adjacent tissue.

In one aspect, the anti-MICA agents that eliminate sMICA producing cells in tumor or tumor-adjacent tissue without increasing or stabilizing sMICA in circulation are advantageous to prevent and/or reduce tumor-mediated immunosuppression. In one aspect provided is a method of treating an individual having a cancer that is poorly responsive to (which has not or which is predicted to not sufficiently or completely respond to) treatment with an immunomodulatory agent, e.g. an agent that mediates ADCC or agent that neutralizes the inhibitory activity of human PD-1, e.g. that inhibits the interaction between PD-1 and PD-L1, for example by binding to PD-L1 or PD-1, the method comprising administering to the individual an anti-MICA agent of the disclosure (optionally further in combination with the immunomodulatory agent.

The methods of treatment the anti-MICA antibody can be used to a treat an individual in combination with a second therapeutic agent, including an anti-cancer agent when used to treat cancer (e.g., chemotherapeutic drugs, tumor vaccines, antibodies that bind to tumor-specific antigens on tumor cells, antibodies that deplete tumor cells, antibodies that potentiate immune responses, etc.).

These and additional advantageous aspects and features of the invention may be further described elsewhere herein.

### BRIEF DESCRIPTION OF THE DRAWINGS

FIG. 1 shows the cytotoxicity of MMAE-immunoconjuates towards HCT116 human colon cancer cells as a function of the concentration of the immunoconjugate, as well as the IC₅₀ (μg/ml) determination of MMAE-immunoconjugates. The MMAE-immunoconjugates did not show an ability at any concentration to deplete HCT116 cells compared to isotype control antibody conjugated to MMAE.

FIG. 2 shows the cytotoxicity of PBD-immunoconjuates towards HCT116 cells as a function of the concentration of the immunoconjugate, as well as the IC₅₀ (μg/ml) determination of PBD-immunoconjugates. PBD-immunoconjugates show strong ability to deplete HCT116 cells.

FIG. 3 shows the anti-tumor activity of anti-MICA-PBD immunoconjugates in a murine model using human colon cancer cells. The PBD-immunoconjugates showed strong anti-tumoral activity even at the low doses, including at each of 0.05 mg/kg, 0.1 mg/kg or 0.2 mg/kg body weight, and effectively prevented increase in tumor volume in all mice and led to elimination of tumors. Of note, in the setting of repeat administration (the 0.05 mg/kg dose), even the lowest dose led to substantially full elimination of tumors.

FIG. 4 shows sMICA levels in mice were engrafted s.c. with a Raji-soluble MICA*001 tumor cell line engineered to secrete the extracellular domain of MICA*001 without expressing MICA*001 at the cell surface. Upon treatment with anti-MICA antibody, more soluble MICA is detected in the anti-MICA antibody-treated group, indicating that soluble MICA half-life in the blood circulation is increased upon binding to anti-MICA antibodies.

FIG. 5 shows the anti-tumor activity of anti-MICA-PBD immunoconjugates in a murine patient-derived xenograft model using human breast cancer cells. The anti-MICA-PBD immunoconjugate (19E9-PBD) showed strong anti-tumoral activity despite a dose of only 0.05 mg/kg once weekly. The standard of care (bevacizumab; Avastin™) showed a partial slowing in growth of tumor volume, and this despite a dose 200 fold higher than that of 19E9-PBD.

FIG. 6 shows sMICA concentration in blood from the PDX mice for each treatment was assessed in blood sampled just before mouse sacrifice. Unlike what can be observed with treatment with anti-MICA antibody as human IgG1 at 10 mg/kg, the anti-MICA-PBD immunoconjugates administered at a dose of 0.05 mg/g did not cause an increase in soluble MICA in circulation.

FIGS. 7, 8, 9 and 10, respectively show that BTG was able to couple the acceptor glutamine at residue 295 substantially completely, obtaining a drug:antibody ratio of 2.0 for each of antibodies humADC2-1, humADC2-2, humADC-3 and humADC2-4. LC/MS analysis of the glycosylated starting antibody humADC2 (top panel), the deglycosylated antibody humADC2 (middle panel), and the antibody humADC2 coupled to NH₂—PEG-N3 (one NH₂-PEG-N3 on each acceptor glutamine per heavy chain).

### DETAILED DESCRIPTION OF THE INVENTION

The immunoconjugates of the invention are able to directly and specifically target and deplete MICA-expressing cells as well as MICB-expressing cells, notably tumor cells. Provided are a number of antibodies for use in such immunoconjugates, including antibodies which bind both Group 1 and Group 2 alleles and moreover across the principal human MICA alleles within these groups, permitting substantially all individuals in the population to be treated, and optionally furthermore advantageously with the same treatment regimen. Optionally, the antibodies bind the α1α2 domain of MICA (the portion of the MICA protein formed from the α1 domain and α2 domain).

MICA (PERB11.1) refers to MHC class I polypeptide-related sequence A (see, e.g., UniProtKB/Swiss-Prot Q29983), its gene and cDNA and its gene product, or naturally occurring variants thereof. Nomenclature of MICA genes and proteins, together with reference to accession number of sequence for different alleles are described in Frigoul A. and Lefranc, M-P. Recent Res. Devel. Human Genet., 3(2005): 95-145 ISBN: 81-7736-244-5, the disclosure of which is incorporated herein by reference. MICA genes and protein sequence, including polymorphisms at the protein and DNA level, are also available from http://www.ebi.ac.uk/ipd/imgt/hla/align.html maintained by Cancer Research UK and the European Bioinformatics Institute (EBI).

The amino acid sequences of MICA were first described in Bahram et al (1994) Proc. Nat. Acad. Sci. 91: 6259-6263 and Bahram et al. (1996) Immunogenetics 44:80-81, the disclosures of which are incorporated herein by reference. The MICA gene is polymorphic, displaying an unusual distribution of a number of variant amino acids in their extracellular α1, α2, and α3 domains. To further define the polymorphism of MICA, Petersdorf et al. (1999) examined its alleles among 275 individuals with common and rare HLA genotypes. The amino acid sequence of the extracellular α1, α2, and α3 domains of human MICA are shown in SEQ ID NOS 1-5. The full MICA sequence further comprises a leader sequence of 23 amino acids, as well as a transmembrane domain and a cytoplasmic domain. The amino acid sequence of extracellular α1, α2, and α3 domains of selected human MICA alleles are shown in SEQ ID NOS 1-5. The amino acid sequence of MICA*001 is shown in SEQ ID NO 1, corresponding to Genbank accession no. AAB41060. The amino acid sequence of human MICA allele MICA*004 is shown in SEQ ID NO 2, corresponding to Genbank accession no. AAB41063. The amino acid sequence of human MICA allele MICA*007 is shown in SEQ ID NO 3, corresponding to Genbank accession no. AAB41066. The amino acid sequence of human MICA allele MICA*008 is shown in SEQ ID NO 4, corresponding to Genbank accession no. AAB41067. The amino acid sequence of human MICA allele MICA*019 is shown in SEQ ID NO 5, corresponding to Genbank accession no. AAD27008.

MICB (also known as PERB11.2) refers to MHC class I polypeptide-related sequence B (See, e.g., UniProtKB/Swiss-Prot Q29980). The amino acid sequence of an exemplary human MICB polypeptide is shown Genbank accession no. CAI18747 (SEQ ID NO: 6).

| MICA | | |
|---|---|---|
| Allele | SEQ ID | Amino acid sequence |
| MICA*001 | 1 | EPHSLRYNLT VLSWDGSVQS GFLTEVHLDG QPFLRCDRQK CRAKPQGQWA |
| | | EDVLGNKTWD RETRDLTGNG KDLRMTLAHI KDQKEGLHSL QEIRVCEIHE |
| | | DNSTRSSQHF YYDGELFLSQ NLETKEWTMP QSSRAQTLAM NVRNFLKEDA |
| | | MKTKTHYHAM HADCLQELRR YLKSGVVLRR TVPPMVNVTR SEASEGNITV |
| | | TCRASGFYPW NITLSWRQDG VSLSHDTQQW GDVLPDGNGT YQTWVATRIC |
| | | QGEEQRFTCY MEHSGNHSTH PVPS |
| MICA*004 | 2 | EPHSLRYNLT VLSWDGSVQS GFLAEVHLDG QPFLRYDRQK CRAKPQGQWA |
| | | EDVLGNKTWD RETRDLTGNG KDLRMTLAHI KDQKEGLHSL QEIRVCEIHE |
| | | DNSTRSSQHF YYDGELFLSQ NVETEEWTVP QSSRAQTLAM NVRNFLKEDA |
| | | MKTKTHYHAM HADCLQELRR YLESSVVLRR RVPPMVNVTR SEASEGNITV |
| | | TCRASSFYPR NITLTWRQDG VSLSHDTQQW GDVLPDGNGT YQTWVATRIC |
| | | QGEEQRFTCY MEHSGNHSTH PVPS |
| MIGA*007 | 3 | EPHSLRYNLT VLSWDGSVQS GFLAEVHLDG QPFLRCDRQK CRAKPQGQWA |
| | | EDVLGNKTWD RETRDLTGNG KDLRMTLAHI KDQKEGLHSL QEIRVCEIHE |
| | | DNSTRSSQHF YYDGELFLSQ NLETEEWTMP QSSRAQTLAM NVRNFLKEDA |
| | | MKTKTHYHAM HADCLQELRR YLKSGVVLRR TVPPMVNVTR SEASEGNITV |
| | | TCRASGFYPW NITLSWRQDG VSLSHDTQQW GDVLPDGNGT YQTWVATRIC |
| | | QGEEQRFTCY MEHSGNHSTH PVPS |
| MICA*008 | 4 | EPHSLRYNLT VLSWDGSVQS GFLAEVHLDG QPFLRYDRQK CRAKPQGQWA |
| | | EDVLGNKTWD RETRDLTGNG KDLRMTLAHI KDQKEGLHSL QEIRVCEIHE |
| | | DNSTRSSQHF YYDGELFLSQ NLETEEWTVP QSSRAQTLAM NVRNFLKEDA |
| | | MKTKTHYHAM HADCLQELRR YLESGVVLRR TVPPMVNVTR SEASEGNITV |
| | | TCRASSFYPR NIILTWRQDG VSLSHDTQQW GDVLPDGNGT YQTWVATRIC |
| | | RGEEQRFTCY MEHSGNHSTH PVPS |
| MICA*019 | 5 | EPHSLRYNLT VLSWDGSVQS GFLAEVHLDG QPFLRYDRQK CRAKPQGQWA |
| | | EDVLGNKTWD RETRDLTGNG KDLRMTLAHI KDQKEGLHSL QEIRVCEIHE |
| | | DNSTRSSQHF YYDGELFLSQ NLETEEWTVP QSSRAQTLAM NVRNFLKEDA |
| | | MKTKTHYHAM HADCLQELRR YLESSVVLRR TVPPMVNVTR SEASEGNITV |
| | | TCRASSFYPR NIILTWRQDG VSLSHDTQQW GDVLPDGNGT YQTWVATRIC |
| | | RGEEQRFTCY MEHSGNHSTH PVPS |
| MICB | 6 | MGLGRVLLFL AVAFPFAPPA AAAEPHSLRY NLMVLSQDGS VQSGFLAEGH |
| | | LDGQPFLRYD RQKRRAKPQG QWAEDVLGAK TWDTETEDLT ENGQDLRRTL |
| | | THIKDQKGGL HSLQEIRVCE IHEDSSTRGS RHFYYDGELF LSQNLETQES |
| | | TVPQSSRAQT LAMNVTNFWK EDAMKTKTHY RAMQADCLQK LQRYLKSGVA |
| | | IRRTVPPMVN VTCSEVSEGN ITVTCRASSF YPRNITLTWR QDGVSLSHNT |
| | | QQWGDVLPDG NGTYQTWVAT RIRQGEEQRF TCYMEHSGNH GTHPVPSGKA |
| | | LVLQSQRTDF PYVSAAMPCF VIIIILCVPC CKKKTSAAEG PELVSLQVLD |
| | | QHPVGTGDHR DAAQLGFQPL MSATGSTGST EGA |

The MICA gene encodes a protein that belongs to the MhcSF and to the IgSF. This protein is a transmembrane MHC-I-alpha-like (I-alpha-like) chain, which comprises three extracellular domains, two distal G-like domains, G-alpha1-like (also referred to as “D1” or “α1”) and G-alpha2-like (also referred to as “D2” or “α2”), and a C-like-domain (also referred to as “D3” or “α3”) proximal to the cell membrane, and three regions, a connecting-region, a transmembrane-region and a cytoplasmic-region (labels according to the IMGT Scientific Chart of the IMGT (international ImMunoGeneTics information System®), http://imgt.org and LeFranc et al. In Silico Biology, 2005; 5:45-60). The MICA mature protein including leader, ECD, TM and CY domains, is made up of 360 to 366 amino acids, the difference arising from a microsatellite polymorphism in the transmembrane region. The α1, α2 and α3 can be defined according to any suitable numbering system (e.g., the IMGT numbering system). In one embodiment, the α1 domain comprises residue positions 1 to 88 of the MICA polypeptide of SEQ ID NO 1; the α2 domain comprises residue positions 89 to 181 of the MICA polypeptide of SEQ ID NO 1; and the α3 domain comprises residue positions 182 to 274 of the MICA polypeptide of SEQ ID NO 1. The α1 and α2 domains each comprise A, B, C and D strands, AB, BC and CD turns, and a helix. The α3 domain comprises A, B, C, D, E, F and G strands, a BC loop, a CD strand, a DE-turn and an FG loop. The MICA protein is highly glycosylated with eight potential glycosylation sites, two in α1, one in α2 and five in the α3 domain, including 0-glycans (N-acetyllactosamine linked to serine or threonine) and/or N-glycans. While MICA is expressed constitutively in certain cells, low levels of MICA expression do not usually give rise to host immune cell attack. However, on MICA is upregulated on rapidly proliferating cells such as tumor cells. MICA is the most highly expressed of all NKG2D ligands, and it has been found across a wide range of tumor types (e.g., carcinomas in general, bladder cancer, melanoma, lung cancer, hepatocellular cancer, glioblastoma, prostate cancer, hematological malignancies in general, acute myeloid leukemia, acute lymphatic leukemia, chronic myeloid leukemia and chronic lymphatic leukemia. Recently, Tsuboi et al. (2011) (EMBO J: 1-13) reported that the 0-glycan branching enzyme, core2 β-1,6-N-acetylglucosaminyltransferase (C2GnT) is active in MICA-expressing tumor cells and that MICA from tumor cells contains core2 O-glycan (an O-glycan comprising an N-acetylglucosamine branch connected to N-acetylgalactosamine).

Bauer et al Science 285: 727-729, 1999 provided a role for MICA as a stress-inducible ligand for NKG2D. As used herein, “MICA” refers to any MICA polypeptide, including any variant, derivative, or isoform of the MICA gene or encoded protein(s) to which they refer. The MICA gene is polymorphic, displaying an unusual distribution of a number of variant amino acids in their extracellular alpha-1, alpha-2, and alpha-3 domains. Various allelic variants have been reported for MICA polypeptides (e.g., MICA), each of these are encompassed by the respective terms, including, e.g., human MICA polypeptides MICA*001, MICA*002, MICA*004, MICA*005, MICA*006, MICA*007, MICA*008, MICA*009, MICA*010, MICA*011, MICA*012, MICA*013, MICA*014, MICA*015, MICA*016, MICA*017, MICA*018, MICA*019, MICA*020, MICA*022, MICA*023, MICA*024, MICA*025, MICA*026, MICA*027, MICA*028, MICA*029, MICA*030, MICA*031, MICA*032, MICA*033, MICA*034, MICA*035, MICA*036, MICA*037, MICA*038, MICA*039, MICA*040, MICA*041, MICA*042, MICA*043, MICA*044, MICA*045, MICA*046, MICA*047, MICA*048, MICA*049, MICA*050, MICA*051, MICA*052, MICA*053, MICA*054, MICA*055, MICA*056 and further MICA alleles MICA*057-MICA*087.

As used herein, “NKG2D” and, unless otherwise stated or contradicted by context, the terms “hNKG2D,” “NKG2-D,” “CD314,” “D12S2489E,” “KLRK1,” “killer cell lectin-like receptor subfamily K, member 1,” or “KLRK1,” refer to a human killer cell activating receptor gene, its cDNA (e.g., GenBank Accession No. NM_007360), and its gene product (Gen Bank Accession No. NP_031386), or naturally occurring variants thereof. In NK and T cells, hNKG2D can form heterodimers or higher order complexes with proteins such as DAP10 (GenBank Accession No. AAG29425, AAD50293). Any activity attributed herein to hNKG2D, e.g., cell activation, antibody recognition, etc., can also be attributed to hNKG2D in the form of a heterodimer such as hNKG2D-DAP10, or higher order complexes with these two (and/or other) components.

The 3D structure of MICA in complex with NKG2D has been determined (see, e.g., Li et al., Nat. Immunol. 2001; 2:443-451; code 1hyr, and in IMGT/3Dstructure-DB (Kaas et al. Nucl. Acids Res. 2004; 32:D208-D210)). When MICA is in complex with a NKG2D homodimer, the residues 63 to 73 (IGMT numbering) of MICA α2 are ordered, adding almost two turns of helix. The two monomers of NKG2D equally contribute to interactions with MICA, and seven positions in each NKG2D monomer interact with one of the MICA α1 or α2 helix domains.

### Definitions

As used in the specification, “a” or “an” may mean one or more. As used in the claim(s), when used in conjunction with the word “comprising”, the words “a” or “an” may mean one or more than one. As used herein “another” may mean at least a second or more.

Where “comprising” is used, this can optionally be replaced by “consisting essentially of”, or optionally by “consisting of”.

Whenever within this whole specification “treatment of a proliferative disease” or “treatment of a tumor”, or “treatment of cancer” or the like is mentioned with reference to anti-MICA binding agent (e.g., antibody), there is meant: (a) method of treatment of a proliferative disease, said method comprising the step of administering (for at least one treatment) an anti-MICA binding agent, (preferably in a pharmaceutically acceptable carrier material) to a warm-blooded animal, especially a human, in need of such treatment, in a dose that allows for the treatment of said disease (a therapeutically effective amount), preferably in a dose (amount) as specified to be preferred hereinabove and herein below; (b) the use of an anti-MICA binding agent for the treatment of a proliferative disease, or an anti-MICA binding agent, for use in said treatment (especially in a human); (c) the use of an anti-MICA binding agent, for the manufacture of a pharmaceutical preparation for the treatment of a proliferative disease, a method of using an anti-MICA binding agent for the manufacture of a pharmaceutical preparation for the treatment of a proliferative disease, comprising admixing an anti-MICA binding agent with a pharmaceutically acceptable carrier, or a pharmaceutical preparation comprising an effective dose of an anti-MICA binding agent that is appropriate for the treatment of a proliferative disease; or (d) any combination of a), b), and c), in accordance with the subject matter allowable for patenting in a country where this application is filed.

The terms “cancer” and “tumor” as used herein are defined as a new growth of cells or tissue comprising uncontrolled and progressive multiplication. In a specific embodiment, upon a natural course the cancer is fatal.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG can readily be employed because they are the most common antibodies in the physiological situation and because they are most easily made in a laboratory setting. The antibody can be a monoclonal antibody. Particularly preferred are humanized, chimeric, human, or otherwise-human-suitable antibodies. “Antibodies” also includes any fragment or derivative of any of the herein described antibodies.

The term “hypervariable region” when used herein refers to the amino acid residues of an antibody that are responsible for antigen binding. The hypervariable region generally comprises amino acid residues from a “complementarity-determining region” or “CDR” (e.g., residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g., residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

When an antibody is said to “compete with” a particular monoclonal antibody (e.g., antibody 19E9), it means that the antibody competes with the monoclonal antibody in a binding assay using either recombinant MICA molecules or surface expressed MICA molecules. For example, if a test antibody reduces the binding of 19E9 to a MICA polypeptide or MICA-expressing cell in a binding assay, the antibody is said to “compete” with 19E9.

The term “affinity”, as used herein, means the strength of the binding of an antibody to an epitope. The affinity of an antibody is given by the dissociation constant K_{D}, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Kₐ is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of surface plasmon resonance (SPR) screening (such as by analysis with a BIAcore™ SPR analytical device).

The term “epitope” refers to an antigenic determinant, and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

The term “depleting”, with respect to MICA-expressing cells means a process, method, or compound that can kill, eliminate, lyse or induce such killing, elimination or lysis, so as to negatively affect the number of MICA-expressing cells present in a sample or in a subject.

The terms “immunoconjugate” and “antibody conjugate” are used interchangeably and refer to an antigen binding agent, e.g., an antibody binding protein or an antibody that is conjugated to another moiety (e.g., a cytotoxic agent). An immunoconjugate comprising an antigen binding agent conjugated to a cytotoxic agent can also be referred to as a “antibody drug conjugate” or an “ADC”.

The term “agent” is used herein to denote a chemical compound, a mixture of chemical compounds, a biological macromolecule, or an extract made from biological materials. The term “therapeutic agent” refers to an agent that has biological activity.

The term “cytotoxic agent” encompasses any compound that can slow down, halt, or reverse the proliferation of cells, decrease their activity in any detectable way, or directly or indirectly kill them. Cytotoxic agents may cause cell death primarily by interfering directly with the cell's functioning, and include, but are not limited to, DNA binding agents, for example DNA minor groove binding agents (e.g., pyrrolobenzodiazepines), spliceosome inhibitors (e.g., thailanstatins), proteasome inhibitors, tubulin polymerization inhibitors (e.g. tubulysins) and topoisomerase inhibitors. Delivery of a cytotoxic agent may be accomplished by administration of a sufficient amount of immunoconjugate comprising an antibody or antigen binding fragment and a cytotoxic agent.

A “humanized” or “human” antibody refers to an antibody in which the constant and variable framework region of one or more human immunoglobulins is fused with the binding region, e.g., the CDR, of an animal immunoglobulin. Such antibodies are designed to maintain the binding specificity of the non-human antibody from which the binding regions are derived, but to avoid an immune reaction against the non-human antibody. Such antibodies can be obtained from transgenic mice or other animals that have been “engineered” to produce specific human antibodies in response to antigenic challenge (see, e.g., Green et al. (1994) Nature Genet 7:13; Lonberg et al. (1994) Nature 368:856; Taylor et al. (1994) Int Immun 6:579, the entire teachings of which are herein incorporated by reference). A fully human antibody also can be constructed by genetic or chromosomal transfection methods, as well as phage display technology, all of which are known in the art (see, e.g., McCafferty et al. (1990) Nature 348:552-553). Human antibodies may also be generated by in vitro activated B cells (see, e.g., U.S. Pat. Nos. 5,567,610 and 5,229,275, which are incorporated in their entirety by reference).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, Md., also referred to as “Kabat” or “Kabat EU”).

The term “antibody-dependent cell-mediated cytotoxicity” or “ADCC” is a term well understood in the art, and refers to a cell-mediated reaction in which non-specific cytotoxic cells that express Fc receptors (FcRs) recognize bound antibody on a target cell and subsequently cause lysis of the target cell. Non-specific cytotoxic cells that mediate ADCC include natural killer (NK) cells, macrophages, monocytes, neutrophils, and eosinophils.

The term “complement-dependent cytotoxicity” or “CDC” is a term well understood in the art, and refers to the ability of a molecule to lyse a target in the presence of complement. The complement activation pathway is initiated by the binding of the first component of the complement system (C1q) to a molecule (e.g., an antibody) complexed with a cognate antigen.

The term “internalization”, used interchangeably with “intracellular internalization”, refers to the molecular, biochemical and cellular events associated with the process of translocating a molecule from the extracellular surface of a cell to the intracellular surface of a cell. The processes responsible for intracellular internalization of molecules are well-known and can involve, inter alia, the internalization of extracellular molecules (such as hormones, antibodies, and small organic molecules); membrane-associated molecules (such as cell-surface receptors); and complexes of membrane-associated molecules bound to extracellular molecules (for example, a ligand bound to a transmembrane receptor or an antibody bound to a membrane-associated molecule). Thus, “inducing and/or increasing internalization” comprises events wherein intracellular internalization is initiated and/or the rate and/or extent of intracellular internalization is increased.

The term “shedding”, when referring to MICA, refers to release of a soluble extracellular domain (ECD) fragment of MICA from the cell surface of a cell which expresses MICA. Such shedding may be caused by proteolytic cleavage (e.g. through the action of matrix metalloproteinases (MMPs), e.g. ADAM10 and/or ADAM17) of cell surface MICA resulting in release of an ECD fragment from the cell surface, or the soluble ECD or fragment thereof may be encoded by an alternate transcript.

The term “transglutaminase”, used interchangeably with “TGase” or “TG”, refers to an enzyme capable of cross-linking proteins through an acyl-transfer reaction between the γ-carboxamide group of peptide-bound glutamine and the ε-amino group of a lysine or a structurally related primary amine such as amino pentyl group, e.g., a peptide-bound lysine, resulting in a ε-(γ-glutamyl)lysine isopeptide bond. TGases include, inter alia, bacterial transglutaminase (BTG) such as the enzyme having EC reference EC 2.3.2.13 (protein-glutamine-γ-glutamyltransferase).

The term “acceptor glutamine residue”, when referring to a glutamine residue of an antibody, means a glutamine residue that is recognized by a TGase and can be cross-linked by a TGase through a reaction between the glutamine and a lysine or a structurally related primary amine such as amino pentyl group. Preferably the acceptor glutamine residue is a surface-exposed glutamine residue.

The term “TGase recognition tag” refers to a sequence of amino acids comprising an acceptor glutamine residue and that when incorporated into (e.g., appended to) a polypeptide sequence, under suitable conditions, is recognized by a TGase and leads to cross-linking by the TGase through a reaction between an amino acid side chain within the sequence of amino acids and a reaction partner. The recognition tag may be a peptide sequence that is not naturally present in the polypeptide comprising the enzyme recognition tag.

By “amino acid modification” herein is meant an amino acid substitution, insertion, and/or deletion in a polypeptide sequence. An example of amino acid modification herein is a substitution. By “amino acid modification” herein is meant an amino acid substitution, insertion, and/or deletion in a polypeptide sequence. By “amino acid substitution” or “substitution” herein is meant the replacement of an amino acid at a given position in a protein sequence with another amino acid. For example, the substitution Y50W refers to a variant of a parent polypeptide, in which the tyrosine at position 50 is replaced with tryptophan. A “variant” of a polypeptide refers to a polypeptide having an amino acid sequence that is substantially identical to a reference polypeptide, typically a native or “parent” polypeptide. The polypeptide variant may possess one or more amino acid substitutions, deletions, and/or insertions at certain positions within the native amino acid sequence.

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. Any compound, e.g., antibody, described herein can optionally be described as being isolated, purified or biologically pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

An antibody that “binds” a polypeptide or epitope designates an antibody that binds said determinant with specificity and/or affinity.

Producing Anti-MICA Antigen binding proteins

Antigen binding domains used in the proteins described herein can be readily derived from any of a variety of immunoglobulin or non-immunoglobulin scaffolds, for example affibodies based on the Z-domain of staphylococcal protein A, engineered Kunitz domains, monobodies or adnectins based on the 10th extracellular domain of human fibronectin Ill, anticalins derived from lipocalins, DARPins (designed ankyrin repeat domains, multimerized LDLR-A module, avimers or cysteine-rich knottin peptides. See, e.g., Gebauer and Skerra (2009) Current Opinion in Chemical Biology 13:245-255, the disclosure of which is incorporated herein by reference. The hypervariable regions, heavy and light chain CDRs, heavy and light chain variable regions, and protein (e.g., antibodies) that comprise them, will bind human (and optionally further non-human primate) MICA expressed on the surface of a cell, e.g., a tumor cell. When used in therapy for the elimination of MICA-expressing tumor cells or for elimination of other MICA-expressing cells that can contribute to cancer progression (e.g. tumor resident macrophages) the antibodies will typically be capable of directly causing the death of MICA-expressing tumor cells when conjugated to a cytotoxic moiety as disclosed herein, and notably without any requirement for ability to mediate ADCC and/or CDC toward the MICA-expressing cells.

In one embodiment, an antigen binding protein or antibody competes for binding to the MICA polypeptide with any one or more of antibodies 6E4, 20C6, 16A8, 9C10, 19E9, 12A10, 10A7, 18E8, 10F3, 15F9 or 14B4. In one embodiment, an antigen binding protein or antibody recognizes, binds to, or has immunospecificity for substantially or essentially the same, or the same, epitope or “epitopic site” on a MICA polypeptide as antibody 6E4, 20C6, 16A8, 9C10, 19E9, 12A10, 10A7, 18E8, 10F3, 15F9 or 14B4.

In one embodiment, the antigen binding protein or antibody binds substantially the same epitope on MICA as antibody 6E4, 20C6, 16A8, 9C10, 19E9, 12A10, 10A7, 18E8, 10F3, 15F9 or 14B4. In another embodiment, the antigen binding protein or antibodies at least partially overlaps, or includes at least one residue in the segment corresponding to residues 1-88, residues 89-181, or residues 182-274 of a MICA polypeptide comprising an amino acid sequence of SEQ ID NOS: 1 to 5. In one embodiment, all key residues of the epitope is in a segment corresponding to residues 1-88, residues 89-181, or residues 182-274 of a MICA polypeptide comprising an amino acid sequence of SEQ ID NOS: 1 to 5. In one embodiment, an antibody binds an epitope spanning the junction of (a) the α1 and/or α2 domain and (b) the α3 domain, wherein all key residues of the epitope is in a segment corresponding to residues 1-181 (e.g., residues 1-88 (optionally 1-85) or 89-181 (optionally 86-181)) of a MICA polypeptide comprising an amino acid sequence of SEQ ID NOS: 1 to 5.

In one embodiment, the antigen binding protein or antibodies bind an epitope comprising 1, 2, 3, 4, 5, 6, 7 or more residues in the segment corresponding to residues 1-88 (optionally 1-85) or residues 89-181 (optionally 86-181) of a MICA polypeptide comprising an amino acid sequence of SEQ ID NOS: 1 to 5. Preferably the residues bound by the antibody are present on the surface of the of the MICA polypeptide, e.g. in a MICA polypeptide expressed on the surface of a cell.

In one embodiment, an antibody binds an epitope comprising 1, 2, 3, 4, 5, or 6 or more residues selected from the group consisting of Q48, W49, E51, D52, V53 and L54.

In one embodiment, an antibody binds an epitope comprising 1, 2, 3, 4, 5, or 6 or more residues selected from the group consisting of N56, K57, T58, R61 and R64.

In one embodiment, an antibody binds an epitope comprising 1, 2, 3, 4, 5, or 6 or more residues selected from the group consisting of K81, D82, Q83, K84, H109, Y111, D113, L116, S133, R134, T137, M140, N141, R143 and N144.

In one embodiment, an antibody binds an epitope comprising 1, 2, 3, 4, 5, or 6 or more residues selected from the group consisting of K81, D82, Q83, K84, H109, Y111, D113, L116, Q131, S132, Q136, M140, N141, R143 and N144.

In one embodiment, an antibody binds an epitope comprising 1, 2, 3, 4, 5, or 6 or more residues selected from the group consisting of E100, D101, N102, S103, T104, R105, N121, E123, T124 and E126.

In one embodiment, the antibodies bind an epitope comprising 1, 2, 3, 4, 5, or 6 or more residues selected from the group consisting of R6, N8, E97, H99, E100, D101, N102, S103, T104, R105, E115, L178, R179 and R180.

Optionally, the epitope of an antigen binding protein or antibody of the invention may be entirely within the α1 and/or α2 domains of MICA. Optionally, further, the antibodies can be characterized as not substantially binding to the α3 domain region required for MICA shedding.

In one embodiment, the antigen binding proteins or antibodies of the invention bind one or more amino acids present on the surface of the MICA polypeptide alleles *001, *004 and *008 (and optionally further *007 and *019).

Binding of anti-MICA agent to cells transfected with the MICA mutants can be measured and compared to the ability of anti-MICA agent to bind wild-type MICA polypeptide (e.g., any one or more of SEQ ID NOS: 1 to 5). A reduction in binding between an anti-MICA agent and a mutant MICA polypeptide means that there is a reduction in binding affinity (e.g., as measured by known methods such FACS testing of cells expressing a particular mutant, or by Biacore testing of binding to mutant polypeptides) and/or a reduction in the total binding capacity of the anti-MICA agent (e.g., as evidenced by a decrease in Bmax in a plot of anti-MICA agent concentration versus polypeptide concentration). A significant reduction in binding indicates that the mutated residue is directly involved in binding to the anti-MICA agent or is in close proximity to the binding protein when the anti-MICA agent is bound to MICA.

In some embodiments, a significant reduction in binding means that the binding affinity and/or capacity between an anti-MICA antibody and a mutant MICA polypeptide is reduced by greater than 40%, greater than 50%, greater than 55%, greater than 60%, greater than 65%, greater than 70%, greater than 75%, greater than 80%, greater than 85%, greater than 90% or greater than 95% relative to binding between the antibody and a wild type MICA polypeptide. In certain embodiments, binding is reduced below detectable limits. In some embodiments, a significant reduction in binding is evidenced when binding of an anti-MICA antibody to a mutant MICA polypeptide is less than 50% (e.g., less than 45%, 40%, 35%, 30%, 25%, 20%, 15% or 10%) of the binding observed between the anti-MICA antibody and a wild-type MICA polypeptide.

In some embodiments, anti-MICA antibodies are provided that exhibit significantly lower binding for a mutant MICA polypeptide in which a residue in a segment corresponding to residues 1-88 (optionally 1-85), residues 89-181 (optionally 86-181), or residues 182-274 (or a subsequence thereof) in a wild-type MICA polypeptide (e.g., comprising a sequence of SEQ ID NOS: 1 to 5) is substituted with a different amino acid. In some embodiments, anti-MICA antibodies are provided that exhibit significantly lower binding for a mutant MICA polypeptide in which a residue in a segment corresponding to residues 1-88 (optionally 1-85), residues 89-181 (optionally 86-181), or residues 182-274 (or a subsequence thereof) in a wild-type MICA polypeptide (e.g., comprising a sequence of SEQ ID NOS: 1 to 5) is substituted with a different amino acid.

Epitopes on MICA protein bound by antibodies 6E4, 20C6, 16A8, 9C10, 19E9, 12A10, 10A7, 18E8, 10F3, 15F9 or 14B4, and mutant anti-MICA proteins to which the antibodies have decreased binding is described in PCT publication no. WO2013/117647. In some embodiments, anti-MICA antibodies are provided that exhibit significantly lower binding for a mutant MICA polypeptide in which a residue selected from the group consisting of R6, N8, Q48, W49, E51, D52, V53, L54, N56, K57, T58, R61, R64, K81, D82, Q83, K84, E97, H99, E100, D101, N102, S103, T104, R105, H109, Y111, D113, E115, L116, N121, E123, T124, E126, Q131, S132, S133, R134, Q136, T137, M140, N141, R143, N144, L178, R179, R180, S224, H225, D226, T227, Q228, Q229, W230 and D232 is substituted with a different amino acid, compared to a wild-type MICA polypeptide.

In some embodiments, anti-MICA antibodies are provided that exhibit significantly lower binding for a mutant MICA polypeptide in which:
(a) 1, 2, 3, 4 or more residues selected from the group consisting of Q48, W49, E51, D52, V53 and L54;
(b) 1, 2, 3, 4 or more residues selected from the group consisting of N56, K57, T58, R61 and R64;
(c) 1, 2, 3, 4 or more residues selected from the group consisting of K81, D82, Q83, K84, H109, Y111, D113, L116, S133, R134, T137, M140, N141, R143 and N144;
(d) 1, 2, 3, 4 or more residues selected from the group consisting of K81, D82, Q83, K84, H109, Y111, D113, L116, Q131, S132, Q136, M140, N141, R143 and N144;
(e) 1, 2, 3, 4 or more residues selected from the group consisting of E100, D101, N102, S103, T104, R105, N121, E123, T124 and E126;
(f) 1, 2, 3, 4 or more residues selected from the group consisting of R6, N8, E97, H99, E100, D101, N102, S103, T104, R105, E115, L178, R179 and R180; or
(g) 1, 2, 3, 4 or more residues selected from the group consisting of S224, H225, D226, T227, Q228, Q229, W230 and D232,
are substituted with a different amino acid, compared to a wild-type MICA polypeptide.

In some embodiments, anti-MICA antibodies are provided that exhibit significantly lower binding for a mutant MICA polypeptide comprising amino acid substitutions at the below listed residues, compared to a wild-type MICA polypeptide (residues listed with reference to SEQ ID NO: 1:

(a) R6 and N8;

(b) N56, K57, T58;

(c) R61 and R64;

(d) K81, D82;

(e) Q83, K84;

(f) E97, H99;

(g) E100, D101, N102;

(h) S103, T104, R105;

(i) D113, E115;

(j) N121, E123;

(k) T124 and E126;

(l) H109, Y111, L116;

(m) Q131, S132, Q136;

(n) S133, R134, T137;

(o) M140, N141, R143 and N144;

(p) S224, H225 and D226;

(q) T227, Q228 and Q229; or

(r) W230 and D232.

In any embodiment, a R6, N8, Q48, W49, E51, D52, V53, L54, N56, K57, T58, R61, R64, K81, D82, Q83, K84, E97, H99, E100, D101, N102, S103, T104, R105, H109, Y111, D113, E115, L116, N121, E123, T124, E126, Q131, S132, S133, R134, Q136, T137, M140, N141, R143, N144, L178, R179, R180, S194, E195, N197, S224, H225, D226, T227, Q228, Q229, W230 or D232 substitution may be specified as being a R6A, NBA, W14A, Q48A, W49S, E51S, D52A, V53S, L54A, N56A, K57S, T58A, R61A, R64A, K81A, D82A, Q83A, K84A, E85A, E97A, H99A, E100A, D101S, N102A, S103A, T104S, R105A, H109A, Y111A, D113A, E115A, L116A, N121A, E123S, T124A, E126A, Q131A, S132A, S133A, R134S, Q136S, T137A, M140S, N141A, R143S, N144A, L178A, R179S, R180A, S224A, H225S, D226A, T227A, Q228S, Q229A, W230A or D232A substitution, respectively.

Antibodies may be produced by a variety of techniques known in the art. Typically, they are produced by immunization of a non-human animal, preferably a mouse, with an immunogen comprising a MICA polypeptide, preferably a human MICA polypeptide. The MICA polypeptide may comprise the full length sequence of a human MICA polypeptide, or a fragment or derivative thereof, typically an immunogenic fragment, i.e., a portion of the polypeptide comprising an epitope exposed on the surface of cells expressing a MICA polypeptide, for example the epitope recognized by the 19E9 antibody. Such fragments typically contain at least about 7 consecutive amino acids of the mature polypeptide sequence, even more preferably at least about 10 consecutive amino acids thereof. Fragments typically are essentially derived from the extra-cellular domain of the receptor. In one embodiment, the immunogen comprises a wild-type human MICA polypeptide in a lipid membrane, typically at the surface of a cell. In a specific embodiment, the immunogen comprises intact cells, particularly intact human cells, optionally treated or lysed. In another preferred embodiment, the polypeptide is a recombinant MICA polypeptide. In a specific embodiment, the immunogen comprises intact MICA-expressing cells.

The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, N.Y. (1988), the entire disclosure of which is herein incorporated by reference).

Antibodies may also be produced by selection of combinatorial libraries of immunoglobulins, as disclosed for instance in (Ward et al. Nature, 341 (1989) p. 544, the entire disclosure of which is herein incorporated by reference).

The identification of one or more antibodies that compete for binding to MICA, with monoclonal antibody 6E4, 20C6, 16A8, 9C10, 19E9, 12A10, 10A7, 18E8, 10F3, 15F9 or 14B4 can be readily determined using any one of a variety of immunological screening assays in which antibody competition can be assessed. Many such assays are routinely practiced and are well known in the art (see, e.g., U.S. Pat. No. 5,660,827, which is incorporated herein by reference).

For example, where the test antibodies to be examined are obtained from different source animals, or are even of a different Ig isotype, a simple competition assay may be employed in which the control (19E9, for example) and test antibodies are admixed (or pre-adsorbed) and applied to a sample containing MICA polypeptides. Protocols based upon western blotting and the use of BIACORE analysis are suitable for use in such competition studies.

In certain embodiments, one pre-mixes the control antibodies (19E9, for example) with varying amounts of the test antibodies (e.g., about 1:10 or about 1:100) for a period of time prior to applying to the MICA antigen sample. In other embodiments, the control and varying amounts of test antibodies can simply be admixed during exposure to the MICA antigen sample. As long as one can distinguish bound from free antibodies (e.g., by using separation or washing techniques to eliminate unbound antibodies) and 19E9 from the test antibodies (e.g., by using species-specific or isotype-specific secondary antibodies or by specifically labeling 19E9 with a detectable label) one can determine if the test antibodies reduce the binding of 19E9 to the antigens. The binding of the (labeled) control antibodies in the absence of a completely irrelevant antibody can serve as the control high value. The control low value can be obtained by incubating the labeled (19E9) antibodies with unlabelled antibodies of exactly the same type (19E9), where competition would occur and reduce binding of the labeled antibodies. A test antibody may for example reduce the binding of 19E9 to MICA antigens by at least about 50%, such as at least about 60%, or more preferably at least about 80% or 90% (e.g., about 65-100%), at any ratio of 19E9:test antibody between about 1:10 and about 1:100. Optionally, such test antibody will reduce the binding of 19E9 to the MICA antigen by at least about 90% (e.g., about 95%).

Competition can also be assessed by, for example, a flow cytometry test. In such a test, cells bearing a given MICA polypeptide can be incubated first with 19E9, for example, and then with the test antibody labeled with a fluorochrome or biotin. The antibody is said to compete with 19E9 if the binding obtained upon preincubation with a saturating amount of 19E9 is about 80%, preferably about 50%, about 40% or less (e.g., about 30%, 20% or 10%) of the binding (as measured by mean of fluorescence) obtained by the antibody without preincubation with 19E9. Alternatively, an antibody is said to compete with 19E9 if the binding obtained with a labeled 19E9 antibody (by a fluorochrome or biotin) on cells preincubated with a saturating amount of test antibody is about 80%, preferably about 50%, about 40%, or less (e.g., about 30%, 20% or 10%) of the binding obtained without preincubation with the test antibody.

A simple competition assay in which a test antibody is pre-adsorbed and applied at saturating concentration to a surface onto which a MICA antigen is immobilized may also be employed. The surface in the simple competition assay is preferably a BIACORE chip (or other media suitable for surface plasmon resonance analysis). The control antibody (e.g., 19E9) is then brought into contact with the surface at a MICA-saturating concentration and the MICA and surface binding of the control antibody is measured. This binding of the control antibody is compared with the binding of the control antibody to the MICA-containing surface in the absence of test antibody. In a test assay, a significant reduction in binding of the MICA-containing surface by the control antibody in the presence of a test antibody indicates that the test antibody recognizes substantially the same region on MICA as the control antibody such that the test antibody “cross-reacts” with the control antibody. A test antibody may for example reduce the binding of control (such as 19E9) antibody to a MICA antigen by at least about 30% or more, preferably about 40%. Optionally, such a test antibody will reduce the binding of the control antibody (e.g., 19E9) to the MICA antigen by at least about 50% (e.g., at least about 60%, at least about 70%, or more). It will be appreciated that the order of control and test antibodies can be reversed: that is, the control antibody can be first bound to the surface and the test antibody is brought into contact with the surface thereafter in a competition assay. Preferably, the antibody having higher affinity for the MICA antigen is bound to the surface first, as it will be expected that the decrease in binding seen for the second antibody (assuming the antibodies are cross-reacting) will be of greater magnitude. Further examples of such assays are provided in, e.g., Saunal (1995) J. Immunol. Methods 183: 33-41, the disclosure of which is incorporated herein by reference.

The antibodies will bind to MICA-expressing cells from an individual or individuals with a cancer characterized by expression of MICA-positive cells, e.g. in the tumor tissue and/or tumor adjacent tissue, i.e., an individual that is a candidate for treatment with one of the herein-described methods using an anti-MICA antibody. Accordingly, once an antibody that specifically recognizes MICA on cells is obtained, it can optionally be tested for its ability to bind to MICA-positive cells (e.g., cancer cells). In particular, prior to treating a patient with one of the present antibodies, one may optionally test the ability of the antibody to bind malignant cells taken from the patient, e.g., in a blood sample or tumor biopsy, to maximize the likelihood that the therapy will be beneficial in the patient.

In one embodiment, the antibodies are validated in an immunoassay to test their ability to bind to MICA-expressing cells, e.g., malignant cells. For example, a blood sample or tumor biopsy is performed and tumor cells are collected. The ability of a given antibody to bind to the cells is then assessed using standard methods well known to those in the art. To assess the binding of the antibodies to the cells, the antibodies can either be directly or indirectly labeled. When indirectly labeled, a secondary, labeled antibody is typically added.

Determination of whether an antibody binds within an epitope region can be carried out in ways known to the person skilled in the art. As one example of such mapping/characterization methods, an epitope region for an anti-MICA antibody may be determined by epitope “foot-printing” using chemical modification of the exposed amines/carboxyls in the MICA protein. One specific example of such a foot-printing technique is the use of HXMS (hydrogen-deuterium exchange detected by mass spectrometry) wherein a hydrogen/deuterium exchange of receptor and ligand protein amide protons, binding, and back exchange occurs, wherein the backbone amide groups participating in protein binding are protected from back exchange and therefore will remain deuterated. Relevant regions can be identified at this point by peptic proteolysis, fast microbore high-performance liquid chromatography separation, and/or electrospray ionization mass spectrometry. See, e.g., Ehring H, Analytical Biochemistry, Vol. 267 (2) pp. 252-259 (1999) Engen, J. R. and Smith, D. L. (2001) Anal. Chem. 73, 256A-265A. Another example of a suitable epitope identification technique is nuclear magnetic resonance epitope mapping (NMR), where typically the position of the signals in two-dimensional NMR spectra of the free antigen and the antigen complexed with the antigen binding peptide, such as an antibody, are compared. The antigen typically is selectively isotopically labeled with 15N so that only signals corresponding to the antigen and no signals from the antigen binding peptide are seen in the NMR-spectrum. Antigen signals originating from amino acids involved in the interaction with the antigen binding peptide typically will shift position in the spectrum of the complex compared to the spectrum of the free antigen, and the amino acids involved in the binding can be identified that way. See, e.g., Ernst Schering Res Found Workshop. 2004; (44): 149-67; Huang et al., Journal of Molecular Biology, Vol. 281 (1) pp. 61-67 (1998); and Saito and Patterson, Methods. 1996 June; 9 (3): 516-24.

Epitope mapping/characterization also can be performed using mass spectrometry methods. See, e.g., Downard, J Mass Spectrom. 2000 April; 35 (4): 493-503 and Kiselar and Downard, Anal Chem. 1999 May 1; 71 (9): 1792-1801. Protease digestion techniques also can be useful in the context of epitope mapping and identification. Antigenic determinant-relevant regions/sequences can be determined by protease digestion, e.g., by using trypsin in a ratio of about 1:50 to MICA or o/n digestion at and pH 7-8, followed by mass spectrometry (MS) analysis for peptide identification. The peptides protected from trypsin cleavage by the anti-MICA binder can subsequently be identified by comparison of samples subjected to trypsin digestion and samples incubated with antibody and then subjected to digestion by e.g., trypsin (thereby revealing a footprint for the binder). Other enzymes like chymotrypsin, pepsin, etc., also or alternatively can be used in similar epitope characterization methods. Moreover, enzymatic digestion can provide a quick method for analyzing whether a potential antigenic determinant sequence is within a region of the MICA polypeptide that is not surface exposed and, accordingly, most likely not relevant in terms of immunogenicity/antigenicity.

Site-directed mutagenesis is another technique useful for elucidation of a binding epitope. For example, in “alanine-scanning”, each residue within a protein segment is re-placed with an alanine residue, and the consequences for binding affinity measured. If the mutation leads to a significant reduction in binding affinity, it is most likely involved in binding. Monoclonal antibodies specific for structural epitopes (i.e., antibodies which do not bind the unfolded protein) can be used to verify that the alanine-replacement does not influence over-all fold of the protein. See, e.g., Clackson and Wells, Science 1995; 267:383-386; and Wells, Proc Natl Acad Sci USA 1996; 93:1-6.

Electron microscopy can also be used for epitope “foot-printing”. For example, Wang et al., Nature 1992; 355:275-278 used coordinated application of cryoelectron micros-copy, three-dimensional image reconstruction, and X-ray crystallography to determine the physical footprint of a Fab-fragment on the capsid surface of native cowpea mosaic virus.

Other forms of “label-free” assay for epitope evaluation include surface plasmon resonance (SPR, BIACORE) and reflectometric interference spectroscopy (RifS). See, e.g., Fägerstam et al., Journal Of Molecular Recognition 1990; 3:208-14; Nice et al., J. Chromatogr. 1993; 646:159-168; Leipert et al., Angew. Chem. Int. Ed. 1998; 37:3308-3311; Kroger et al., Biosensors and Bioelectronics 2002; 17:937-944.

It should also be noted that an antibody binding the same or substantially the same epitope as an antibody can be identified in one or more of the exemplary competition assays described herein.

Upon immunization and production of antibodies in a vertebrate or cell, particular selection steps may be performed to isolate antibodies as claimed. In this regard, in a specific embodiment, provided are methods of producing such antibodies, comprising: (a) immunizing a non-human mammal with an immunogen comprising a MICA polypeptide; and (b) preparing antibodies from said immunized animal; and (c) selecting antibodies from step (b) that are capable of binding MICA.

In one aspect an antibody can have an average disassociation constant (K_{D}) of no more than 1×10⁻⁸ M, optionally less than 1×10⁻⁹ M with respect to human MICA, as determined by, e.g., surface plasmon resonance (SPR) screening (such as by analysis with a BIAcore™ SPR analytical device). In a more particular exemplary aspect, provided are anti-MICA antibodies that have a KD of about 1×10⁻⁸ M to about 1×10⁻¹⁰ M, or about 1×10⁻⁹ M to about 1×10⁻¹¹ M, for MICA. SPR can be carried out for examples according to the conditions described in PCT publication no. WO2013/117647.

Antibodies can be characterized for example by a mean KD of no more than about (i.e., better affinity than) 100, 60, 10, 5, or 1 nanomolar, preferably sub-nanomolar or optionally no more than about 500, 200, 100 or 10 picomolar. KD can be determined for example for example by immobilizing recombinantly produced human MICA proteins on a chip surface, followed by application of the antibody to be tested in solution. In one embodiment, the method further comprises a step (d), selecting antibodies from (b) that are capable of competing for binding to MICA with antibody 19E9.

In one aspect of any of the embodiments, the antibodies prepared according to the present methods are monoclonal antibodies. In another aspect, the non-human animal used to produce antibodies is a mammal, such as a rodent, bovine, porcine, fowl, horse, rabbit, goat, or sheep. Antibodies of the invention can optionally be specified to be antibodies other than any of antibodies 6E4, 20C6, 16A8, 9C10, 19E9, 12A10, 10A7, 18E8, 10F3, 15F9 or 1464, or derivatives thereof, e.g., that comprise their respective heavy and light chain CDRs or the antigen binding region in whole or in part.

DNA encoding an antibody that binds an epitope present on MICA polypeptides is isolated from a hybridoma and placed into an expression vector(s), which is then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. As described elsewhere in the present specification, such DNA sequences can be modified for any of a large number of purposes, e.g., for humanizing antibodies, producing fragments or derivatives, or for modifying the sequence of the antibody, e.g., in the antigen binding site in order to optimize the binding specificity of the antibody. In one embodiment, provided is an isolated nucleic acid sequence encoding a light chain and/or a heavy chain of an antibody, as well as a recombinant host cell comprising (e.g., in its genome) such nucleic acid.

Once an antibody is obtained it will generally be assessed for its activity. The antibody can be conjugated to a cytotoxic agent, e.g., as further described herein, and assessed for the ability to deplete a MICA-expressing target cell (e.g., a HCT116 cell) and/or to bind a MICA-expressing target cell, e.g. a C1R (or other) cell made to express at its surface a MICA*001 polypeptide, a C1R (or other) cell made to express at its surface a MICA*004 polypeptide, a C1R (or other) cell made to express at its surface a MICA*007 polypeptide, and/or a C1R (or other) cell made to express at its surface a MICA*008 polypeptide, and optionally further a C1R (or other) cell made to express at its surface a MICAB polypeptide.

The antibody can be characterized by a binding affinity (K_{D}), optionally wherein binding affinity is monovalent or bivalent, for a human MICA and/or MICB polypeptide (e.g. any one or more or all MICA and/or MICB alleles referred to herein, e.g. a MICA*001 polypeptide, a MICA*004 polypeptide, MICA*007 polypeptide and a MICA*008 polypeptide) of less than 10⁻⁹ M, preferably less than 10⁻¹⁰ M, preferably less than 10⁻¹¹ M, preferably less than 10⁻¹² M, or preferably less than 10⁻¹³M. Preferably the antibody is a tetrameric antibody comprising two Ig heavy chains and two Ig light chains and the K_{D} is bivalent. Optionally, the antibody has an EC₅₀ for binding to cells made to express a MICA*001 polypeptide, cells made to express a MICA*004 polypeptide, cells made to express a MICA*007 polypeptide and cells made to express a MICA*008 polypeptide (and optionally further to cells made to express a MICB polypeptide), of no more than 2, 1, 0.5 or 0.1 μg/ml. Examples of suitable cells are C1R-MICA cells. Optionally, EC₅₀ for binding to cells is determined according to flow cytometry and EC₅₀ is computed using a 4-parameter model, optionally EC₅₀ for binding to cells is determined as described in PCT publication no. WO2013/117647, the disclosure of which is incorporated herein by reference.

In one embodiment, the cells made to express MICA or MICB are C1R transfectant cells prepared as described in Salih et al. (2003) Blood 102(4): 1389-91396, the disclosure of which is incorporated herein by reference. For example, C1R cells (ATCC under reference CRL-1993™), widely used transfection recipients, transfected with RSV.5neo described in Long E. O. et al., Hum. Immunol. 31:229-235(1991); the nucleic acid sequence is available in GenBank (NCBI) under Accession number M83237 (record dated 2001).

Testing the ability of an immunoconjugate to deplete a target cell (e.g. by direct cytotoxicity) can be carried out by any of a number of assays, known in the art, for determining whether an immunoconjugate exerts a cytostatic or cytotoxic effect on a desired cell line. For example, the cytotoxic or cytostatic activity of an immunoconjugate can be measured by: exposing mammalian cells expressing MICA in a cell culture medium; culturing the cells for a period from about 6 hours to about 1 week; and assessing cell viability.

In one example, cell confluence in wells of 96 well plates is measured over time using the IncuCyte Zoom apparatus (Essen Biosciences, Ann Arbor, Mich.), wherein target cells (e.g. tumor cells) are seeded in culture medium and incubated for 4 hours at 37° C. in the presence of different final concentrations of immunoconjugates, followed by incubation of cells at 37° C. for one week, during which time four pictures/wells are taken every 6 hours. Percentage of cell confluence values are obtained using IncuCyte Zoom software and Area Under the Curve vs. concentration of immunoconjugates (Log [ADCs]) as well as EC₅₀ values are obtained using GraphPad Prism software.

Generally any suitable cell-based in vitro assays can be used to measure viability (proliferation) and/or induction of apoptosis (caspase activation). For example, cell viability can be measured by assessing ATP production by MICA-expressing cells in culture.

In other examples, a thymidine incorporation assay may be used. For example, cancer cells expressing MICA at a density of 5,000 cells/well of a 96-well plated can be cultured for a 72-hour period and exposed to 0.5 μCi of ³H-thymidine during the final 8 hours of the 72-hour period. The incorporation of ³H-thymidine into cells of the culture is measured in the presence and absence of the immunoconjugate.

In another example determining cytotoxicity, necrosis or apoptosis (programmed cell death) can be measured. Necrosis is typically accompanied by increased permeability of the plasma membrane; swelling of the cell, and rupture of the plasma membrane. Apoptosis is typically characterized by membrane blebbing, condensation of cytoplasm, and the activation of endogenous endonucleases. Determination of any of these effects on cancer cells indicates that an immunoconjugate is useful in the treatment of cancers.

Cell viability can also be measured by determining in a cell the uptake of a dye such as neutral red, trypan blue, or ALAMAR™ blue. In such an assay, the cells are incubated in media containing the dye, the cells are washed, and the remaining dye, reflecting cellular uptake of the dye, is measured spectrophotometrically. The protein-binding dye sulforhodamine B (SRB) can also be used to measure cytotoxicity. Alternatively, a tetrazolium salt, such as MTT or WST, is used in a quantitative colorimetric assay for mammalian cell survival and proliferation by detecting living, but not dead, cells.

Apoptosis can be quantitated by measuring, for example, DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays. Apoptosis can also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). Cells also can be labeled with a DNA dye (e.g., acridine orange, ethidium bromide, or propidium iodide) and the cells observed for chromatin condensation and margination along the inner nuclear membrane. Other morphological changes that can be measured to determine apoptosis include, e.g., cytoplasmic condensation, increased membrane blebbing, and cellular shrinkage.

The presence of apoptotic cells can be measured in both the attached and “floating” compartments of the cultures. For example, both compartments can be collected by removing the supernatant, trypsinizing the attached cells, combining the preparations following a centrifugation wash step (e.g., 10 minutes at 2000 rpm), and detecting apoptosis (e.g., by measuring DNA fragmentation). (See, e.g., Piazza et al., (1995), Cancer Research 55: 3110-16).

In any embodiment, direct depletion of or cytotoxicity towards MICA-expressing target cells can be specified to be in the absence of immune effector cells, and/or at a concentration below that at which the antibody is capable of exerting significant ADCC and/or CDC.

Exemplary Antibody sequences

The amino acid sequence of the heavy and light chain variable region of exemplary antibodies 6E4, 20C6, 16A8, 9C10, 19E9 (including exemplary humanized variants 19E9-1, 19E9-2 and 19E9-3), 12A10, 10A7, 18E8, 10F3, 15F9 and 14B4 are shown in Table 1. In a specific embodiment, the immunoconjugate comprises an antibody that binds essentially the same epitope or determinant as any of the monoclonal antibodies 6E4, 20C6, 16A8, 9C10, 19E9, 12A10, 10A7, 18E8, 10F3, 15F9 or 1464; optionally the antibody comprises an antigen binding region (e.g. hypervariable region) of antibody 6E4, 20C6, 16A8, 9C10, 19E9, 12A10, 10A7, 18E8, 10F3, 15F9 or 1464. In any of the embodiments herein, antibody 6E4, 20C6, 16A8, 9C10, 19E9, 12A10, 10A7, 18E8, 10F3, 15F9 or 14B4 can be characterized by the amino acid sequences thereof (e.g. CDR sequences) and/or nucleic acid sequence encoding it. In one embodiment, the monoclonal antibody comprises the Fab or F(ab′)₂ portion. According to one embodiment, an antibody comprises the three CDRs of the heavy chain variable region of 6E4, 20C6, 16A8, 9C10, 19E9, 12A10, 10A7, 18E8, 10F3, 15F9 or 1464, and the three of the CDRs of the light chain variable region of the respective 6E4, 20C6, 16A8, 9C10, 19E9, 12A10, 10A7, 18E8, 10F3, 15F9 or 14B4 antibody. Optionally any one or more of said light or heavy chain CDRs may contain one, two, three, four or five or more amino acid modifications (e.g. substitutions, insertions or deletions). According to one embodiment, an antibody comprises a VH domain of antibody 19E9-1, 19E9-2 or 19E9-1 antibody and a VL domain of the respective 19E9-1, 19E9-2 or 19E9-1 antibody. Optionally, provided is an antibody where any of the light and/or heavy chain variable regions comprising part or all of an antigen binding region of the antibody are fused to an immunoglobulin constant region of the human IgG type, optionally a human constant region, optionally a human IgG1, IgG2, IgG3 or IgG4 isotype, comprising amino acid modification(s) that results in reduced binding to human CD16A, CD16B, CD32A, CD32B and/or CD64 (e.g. compared to the same antibody with a wild-type constant region).

In another aspect, the antibody comprises: a HCDR1 region comprising an amino acid sequence SDYAWN, GYSITSD or GYSITSDYAWN, or a sequence of at least 4, 5, 6, 7, 8, 9 or 10 contiguous amino acids thereof, wherein one or more of these amino acids may be substituted by a different amino acid; a HCDR2 region comprising an amino acid sequence FVSYSGTTKYNPSLKS or FVSYSGTTK, or a sequence of at least 4, 5, 6, 7, 8, 9 or 10 contiguous amino acids thereof, wherein one or more of these amino acids may be substituted by a different amino acid; a HCDR3 region comprising an amino acid sequence GYGFDY, or a sequence of at least 4, 5, 6, 7, 8, 9 or 10 contiguous amino acids thereof, wherein one or more of these amino acids may be substituted by a different amino acid; a LCDR1 region comprising an amino acid sequence SATSSISSIYFH, or a sequence of at least 4, 5, 6, 7, 8, 9 or 10 contiguous amino acids thereof, wherein one or more of these amino acids may be substituted by a different amino acid; a LCDR2 region comprising an amino acid sequence RTSNLAS, or a sequence of at least 4, 5, 6, 7, 8, 9 or 10 contiguous amino acids thereof, wherein one or more of these amino acids may be substituted by a different amino acid; a LCDR3 region comprising an amino acid sequence QQGTTIPFT, or a sequence of at least 4, 5, 6, 7, 8, 9 or 10 contiguous amino acids thereof, wherein one or more of these amino acids may be deleted or substituted by a different amino acid.

In a specific embodiment, the immunoconjugate comprises an antibody that binds human MICA, comprising:

(a) the heavy chain CDR 1, 2 and 3 (HCDR1, HCDR2, HCDR3) amino acid sequences as shown in Table 2, optionally wherein one, two, three or more amino acids in a CDR may be substituted by a different amino acid; and/or

(b) the respective light chain CDR 1, 2 and 3 (LCDR1, LCDR2, LCDR3) amino acid sequences as shown in in Table 2, optionally wherein one, two, three or more amino acids in a CDR may be substituted by a different amino acid.

In a specific embodiment, the immunoconjugate comprises an antibody that binds human MICA, comprising: the heavy chain CDR 1, 2 and 3 (HCDR1, HCDR2, HCDR3) amino acid sequences as shown in in Table 2, optionally wherein one two, three or more amino acids in a CDR may be substituted by a different amino acid; and the respective light chain CDRs 1, 2 and 3 (LCDR1, LCDR2, LCDR3) amino acid sequences as shown in in Table 2, optionally wherein one, two, three or more amino acids in a CDR may be substituted by a different amino acid.

In a specific embodiment, the immunoconjugate comprises an antibody that binds human MICA, comprising: the heavy chain variable region which is at least 60%, 70%, 80%, 85%, 90% or 95% identical to the heavy chain variable region having an amino acid sequence of Table 1, wherein one, two, three or more amino acids may be substituted by a different amino acid; and the light chain variable region which is at least 60%, 70%, 80%, 85%, 90% or 95% identical to the respective light chain variable region having an amino acid sequence of Table 1, wherein one, two, three or more amino acids may be substituted by a different amino acid.

In any aspect of any of the embodiments herein, any of the CDRs 1, 2 and 3 of the heavy and light chains may be characterized by a sequence of at least 4, 5, 6, 7, 8, 9 or 10 contiguous amino acids thereof, and/or as having an amino acid sequence that shares at least 50%, 60%, 70%, 80%, 85%, 90% or 95% sequence identity with the particular CDR or set of CDRs of the VH/VL of Table 1 or as set out in Table 2.

In other examples, the immunoconjugate comprises an antibody that competes for binding to a MICA polypeptide with any one or any combination of monoclonal antibodies 6E4, 20C6, 16A8, 9C10, 19E9, 12A10, 10A7, 18E8, 10F3, 15F9 and 14B4 (e.g. an antibody having the VH and the VL amino acid sequence listed in the Table 1 below).

In another embodiment, the immunoconjugate comprises an antibody that binds human MICA, comprising: the heavy chain CDR 1, 2 and 3 (HCDR1, HCDR2, HCDR3) amino acid sequences of the 6E4, 20C6, 16A8, 9C10, 19E9, 12A10, 10A7, 18E8, 10F3, 15F9 or 14B4 VH domain as shown in Table 1, optionally wherein one two, three or more amino acids in a CDR may be substituted by a different amino acid; and the light chain CDRs 1, 2 and 3 (LCDR1, LCDR2, LCDR3) amino acid sequences of the respective 6E4, 2006, 16A8, 9C10, 19E9, 12A10, 10A7, 18E8, 10F3, 15F9 or 14B4 VL domain shown in Table 1, optionally wherein one, two, three or more amino acids in a CDR may be substituted by a different amino acid.

The sequences of the CDRs can be determined according to any desired numbering scheme, for example AbM (Oxford Molecular's AbM antibody modelling software definition), Kabat and Chothia definitions systems.

TABLE 1

| Antibody | | |
|---|---|---|
| portion | SEQ ID NO | Sequence |
| 6E4 VH | 7 | EVQLVESGGALVKPGGSLKLSCAASGFTFSYYAMSWVRQTPEKRLEWVATI |
| | | WRGGNYIYYTDSVKGRFTISRDNAKNTLYLQMTSLRSEDTAMFYCASISDY |
| | | DGAWLAYWGQGTLVTV |
| 6E4 VL | 8 | EVLMTQTPLSLPVSLGDQASISCRSSQSIIHTNGNTYLEWYLQKPGQSPKL |
| | | LIYKISNRFSGVPDRFSGSGSGTDFTLKISRVEAEDLGVYYCFQGSHVPWT |
| | | FGGGTKLEIK |
| 20C6 VH | 9 | QITLKESGPGILKPSQTLSLTCSFSGFSLSTSGMGVGWIRQPSGKGLEWLA |
| | | HIWWDDDKYYNPSLKSQLTISKDTSRNQVFLRITSVDTADTATYYCARRTQ |
| | | GYFDYWGQGTTLVTVSS |
| 20C6 VL | 10 | DIVMTQSPATLSVTPGDRVSLSCRASQSISDYLHWYQQKSHESPRLLIKYA |
| | | SQSISGIPSRFSGSGSGSDFTLSINSVEPEDVGVYYCQNGHSFPWTFGGGT |
| | | KLEIK |
| 16A8 VH | 11 | EVQLVESGGGLVKPGGSLKLSCAASGFTFSRYAMSWVRQTPEKRLEWVATI |
| | | FSGGSYTYYPDSVKGRFTISRDNANNTLYLQMSSLKAEDTAMYFCARPNWE |
| | | RTFDYWGQGTTLTVSS |
| 16A8 VL | 12 | DIVMTQSPSSLAMSVGQKVTMSCKSSQSLLNSSNQKNYLAWYQQKPGQSPK |
| | | LLVYFASTRESGVPDRFMGSGSGTDFTLTISSVQAEDLADYFCQQHYSTPP |
| | | TFGGGTKLEIK |
| 19E9 VH | 13 | MRVLILLWLFTAFPGLLSDVQLQESGPGLVKPSQSLSLTCTVTGYSITSDY |
| | | AWNWIRQFPGNKLEWMGFVSYSGTTKYNPSLKSRISITRDTSENQFFLQLN |
| | | SVTSEDTATYYCARGYGFDYWGQGTTLTVSS |
| 19E9 VL | 14 | EIVLTQSPTTMAASPGEKITITCSATSSISSIYFHWYQQRPGFSPKLLIYR |
| | | TSNLASGVPARFSGSGSGTSYSLTIGTMEAEDVATYYCQQGTTIPFTFGSG |
| | | TKLEIK |
| 19E9-1 VH | 15 | QVQLQESGPGLVKPSETLSLTCTVSGYSITSDYAWNWIRQPPGKGLEWIGF |
| | | VSYSGTTKYNPSLKSRVTISRDTSKNQFSLKLSSVTAADTAVYYCARGYGF |
| | | DYWGQGTTVTVSS |
| 19E9-1 VL | 16 | EIVLTQSPATLSLSPGERATLSCSATSSISSIYFHWYQQKPGQAPRLLIYR |
| | | TSNLASGIPARFSGSGSGTDYTLTISSLEPEDFAVYYCQQGTTIPFTFGQG |
| | | TKLEIK |
| 19E9-2V H | 17 | QVQLQESGPGLVKPSETLSLTCTVSGYSITSDYAWNWIRQPPGKGLEWIGF |
| | | VSYSGTTKYNPSLKSRVTISRDTSKNQFSLKLSSVTAADTAVYYCARGYGF |
| | | DYWGQGTTVTVSS |
| 19E9-2 VL | 18 | EIVLTQSPATLSLSPGERATLSCSATSSISSIYFHWYQQKPGQAPRLLIYR |
| | | TSNLASGIPARFSGSGSGTSYTLTISSLEPEDFAVYYCQQGTTIPFTFGQG |
| | | TKLEIK |
| 19E9-3 VH | 19 | QVQLQESGPGLVKPSETLSLTCTVSGYSITSDYAWNWIRQPPGKGLEWIGF |
| | | VSYSGTTKYNPSLKSRVTISRDTSKNQFSLKLSSVTAADTAVYYCARGYGF |
| | | DYWGQGTTVTVSS |
| 19E9-3 VL | 20 | EIVLTQSPATLSLSPGERATLSCSATSSISSIYFHWYQQKPGQAPRLLIYR |
| | | TSNLASGIPARFSGSGSGTDYTLTISSLEPEDVAVYYCQQGTTIPFTFGQG |
| | | TKLEIK |
| 9C10 VH | 21 | QVQLQQPGAELVRPGTSVNLSCKASGYSFTRYWMNWVKQRPGQGLEWIGMI |
| | | HPSDSETRLNQKFKDKATLTVDKSSSTAYMQLSSPTSEDSAVYYCGYGNFF |
| | | YVMDYWGQGTSVTVSS |
| 9C10 VL | 22 | DILLTQSPAILSVSPGERVSFSCRASQSIGTSIHWYQQRTNGSPRLLIKFA |
| | | SESISGIPSRFSGSGSGTDFTLNINSVESEDIADYYCQQSNFWPFTFGSGT |
| | | KLEVK |
| 12A10 VH | 23 | QVQLQQSGADLVRPGASVRLSCRASGYSFTNYWMNWVKQRPGQGLEWIGMI |
| | | HPSDSETRLNQKFKDKATLTVDKSSNTAYMQLSSPTSEDSAIYYCARDDFF |
| | | TMDYWGQGTSVTVSSASTK |
| 12A10 VL | 24 | DILLTQSPAILSVSPGERVSFSCRASQNIVTSIHWYQQSTNGSPRLLIKYA |
| | | SESISGIPSRFSGSGSGTDFTLTINSVESEDVADYYCQQSNIWPLTFGAGT |
| | | KLELK |
| 10A7 VH | 25 | QVTLKESGPGILKPSQTLSLTCSFSGFSLSTSGMGVGWIRQPSGKGLEWLA |
| | | HIWWDDDRYYNPSLKSQLTISKDTSRNQVFLKITSVDTADTATYYCARRLN |
| | | GYFDYWGQGTTLTVSSASTK |
| 10A7 VL | 26 | DIVMTQSPATLSVTLGDRVSLSCRASQSISDYLHWYQQKSHESPRLLIKYA |
| | | SQSISGIPSRFSGSGSGSDFTLSINSVEPEDVGVYYCQNGHSFPFTFGSGT |
| | | KLEIK |
| 18E8 VH | 27 | DVQLQESGPDLVNPSQSLSLICTVTGYSITSDYSWHWIRQFPGNKLEWMGN |
| | | IHYSGRINYNPSLRSRISITRDTSKNQFFLQLISVTTEDTATYYCATRRTF |
| | | GNFEDYWGQGTTLTVSSASTK |
| 18E8 VL | 28 | QIVLSQSPATLSVSPGEKVTMTCRSSSSVNYMHWYQQKPGSSPKPWIYATS |
| | | TLASGVPARFSGSGSGTSYSLTISRVEAEDAATYYCQQWSSNPLTFGAGTK |
| | | LELK |
| 10F3 VH | 29 | MEWSWVFLFFLSVTTGVHSQVQLQQSAAELARPGASVKMSCKASGYTFTSY |
| | | TMHWVKQRPGQGLEWIGYINPSSGYTEYNQKFKDKTTLTVDKSSTTSYMQL |
| | | SSLTSDNSAVYYCARGGDWDVDWFVYWGQGTLVTVSAASTK |
| 10F3 VL | 30 | QIVLTQSPAIMSASPGEKVTITCSASSSISYMHWFQQKPGTSPKLWIYSTS |
| | | KLASGVPARFSGSGSGTSHSLTISRMEAEDAATYYCQHRSTYPFTFGSGTK |
| | | LEIK |
| 15F9 VH | 31 | DVQLQESGPDLVKPSQSLSLTCTVTGYSITSGYSWHWIRQFPGNKLEWMGF |
| | | IHYSGSTDYNPSLKSRISLTRDTSKNQFFLQLNSVSTEDTATYYCAKDYGH |
| | | WYFDVWGAGTTVTVSSASTK |
| 15F9 VL | 32 | MSVPTQVLGLLLLWLTDARCSIVMTQTPKFLLVSAGDRVTITCKASQSVSY |
| | | DVAWYQQKPGQSPKLLIFYASNRYTGVPARFTGSGYGTDFTFTISTVQAED |
| | | LAVYFCQQDYSSLTFGAGTKLELK |
| 14B4 VH | 33 | QVQLQQPGAELVRPGASVKLSCKASGYSFTSYWMNWMKQRPGQGLEWIGMI |
| | | HPSDSETRLNQKFKDKATLTVDKSSSTAYMQLNSPTSEDSAVYYCAREMGP |
| | | YTLDYWGQGTSVTVSSASTK |
| 14B4 VL | 34 | DILLTQSPAILSVSPGARVSFSCRASQNIDTSIHWYQQRTNGSPRLLIKYA |
| | | SESISGIPSRFSGSGSGTDFTLSINSVESEDIADYYCQQSNYWPLTFGAGT |
| | | KLELK |

In another embodiment, the immunoconjugate comprises an antibody that binds human MICA, comprising: the Kabat heavy chain CDR 1, 2 and 3 (HCDR1, HCDR2, HCDR3) amino acid sequences of antibody 6E4, 20C6, 16A8, 9C10, 19E9, 12A10, 10A7, 18E8, 10F3, 15F9 or 14B4 as shown in Table 2, optionally wherein one two, three or more amino acids in a CDR may be substituted by a different amino acid; and the Kabat light chain CDRs 1, 2 and 3 (LCDR1, LCDR2, LCDR3) amino acid sequences of the respective 6E4, 20C6, 16A8, 9C10, 19E9, 12A10, 10A7, 18E8, 10F3, 15F9 or 14B4 as shown in Table 2, optionally wherein one, two, three or more amino acids in a CDR may be substituted by a different amino acid.

TABLE 2

| | HCDR1 | | HCDR2 | | HCDR2 | |
|---|---|---|---|---|---|---|
| mAb | SEQ ID | Sequence | SEQ ID | Sequence | SEQ ID | Sequence |
| 6E4 | 35 | SYYAMS | 36 | TISRGGNYIYYTDSVKG | 37 | ISDYDGAWLAY |
| 20C6 | 41 | TSGMGVG | 42 | HIWWDDDKYYNPSLK | 43 | RTQGYFDY |
| 16A8 | 47 | RYAMS | 48 | TIFSGGSYTYYPDSV | 49 | PNWERTFDY |
| 19E9 | 53 | SDYAWN | 54 | FVSYSGTTKYNPSLKS | 55 | GYGFDY |
| 9C10 | 59 | RYWMN | 60 | MIHPSDSETRLNQKFKD | 61 | GNFFYVMDY |
| 12A10 | 65 | NYWMN | 66 | MIHPSDSETRLNQKFKD | 67 | DDFFTMDY |
| 10A7 | 71 | TSGMGVG | 72 | HIWWDDDRYYNPSLKS | 73 | RLNGYFDY |
| 18E8 | 77 | SDYSWH | 78 | NIHYSGRINYNPSLRS | 79 | RRTFGNFEDY |
| 10F3 | 83 | SYTMH | 84 | YINPSSGYTEYNQKFKD | 85 | GGDWDVDWFVY |
| 15F9 | 89 | SGYSWH | 90 | FIHYSGSTDYNPSLKS | 91 | DYGHWYFDV |
| 14B4 | 95 | SYWMN | 96 | MIHPSDSETRLNQKFKD | 97 | EMGPYTLDY |
| | LCDR1 | | LCDR2 | | LCDR3 | |
| mAb | SEQ ID | Sequence | SEQ ID | Sequence | SEQ ID | Sequence |
| 6E4 | 38 | RSSQSIIHTNGNTYLE | 39 | KISNRFS | 40 | FQGSHVPWT |
| 20C6 | 44 | RASQSISDYLH | 45 | YASQSIS | 46 | QNGHSFPWT |
| 16A8 | 50 | KSSQSLLNSSNQKNYL | 51 | FASTRES | 52 | QQHYSTPPT |
| 19E9 | 56 | SATSSISSIYFH | 57 | RTSNLAS | 58 | QQGTTIPFT |
| 9C10 | 62 | RASQSIGTSIH | 63 | ASESISG | 64 | QQSNFWPFT |
| 12A10 | 68 | RASQNIVTSIH | 69 | YASESIS | 70 | QQSNIWPLT |
| 10A7 | 74 | RASQSISDYLH | 75 | YASQSIS | 76 | QNGHSFPFT |
| 18E8 | 80 | RSSSSVNYMH | 81 | ATSTLAS | 82 | QQWSSNPLT |
| 10F3 | 86 | SASSSISYMH | 87 | STSKLAS | 88 | QHRSTYPFT |
| 15F9 | 92 | KASQSVSYDVA | 93 | YASNRYT | 94 | QQDYSSLT |
| 14B4 | 98 | RASQNIDTSIH | 99 | YASESIS | 100 | QQSNYWPLT |

Fragments and derivatives of antibodies (which are encompassed by the term “antibody” or “antibodies” as used in this application, unless otherwise stated or clearly contradicted by context), can be produced by techniques that are known in the art. “Fragments” comprise a portion of the intact antibody, generally the antigen binding site or variable region. Examples of antibody fragments include Fab, Fab′, Fab′-SH, F (ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Included, inter alia, are a nanobody, domain antibody, single domain antibody or a “dAb”.

In one embodiment, the antibody is humanized. “Humanized” forms of antibodies are specific chimeric immunoglobulins, immunoglobulin chains or fragments thereof (such as Fv, Fab, Fab′, F (ab′) 2, or other antigen-binding subsequences of antibodies) which contain minimal sequence derived from the murine immunoglobulin. For the most part, humanized antibodies are human immunoglobulins (recipient antibody) in which residues from a complementary-determining region (CDR) of the recipient are replaced by residues from a CDR of the original antibody (donor antibody) while maintaining the desired specificity, affinity, and capacity of the original antibody.

In some instances, Fv framework residues of the human immunoglobulin may be replaced by corresponding non-human residues. Furthermore, humanized antibodies can comprise residues that are not found in either the recipient antibody or in the imported CDR or framework sequences. These modifications are made to further refine and optimize antibody performance. In general, the humanized antibody will comprise substantially all of at least one, and typically two, variable domains, in which all or substantially all of the CDR regions correspond to those of the original antibody and all or substantially all of the FR regions are those of a human immunoglobulin consensus sequence. The humanized antibody optimally also will comprise at least a portion of an immunoglobulin constant region (Fc), typically that of a human immunoglobulin. For further details see Jones et al., Nature, 321, pp. 522 (1986); Reichmann et al, Nature, 332, pp. 323 (1988); Presta, Curr. Op. Struct. Biol., 2, pp. 593 (1992); Verhoeyen et Science, 239, pp. 1534; and U.S. Pat. No. 4,816,567, the entire disclosures of which are herein incorporated by reference.

The choice of human variable domains, both light and heavy, to be used in making the humanized antibodies is very important to reduce antigenicity. According to the so-called “best-fit” method, the sequence of the variable domain of an antibody is screened against the entire library of known human variable-domain sequences. The human sequence which is closest to that of the mouse is then accepted as the human framework (FR) for the humanized antibody (Sims et al., J. Immunol. 151, pp. 2296 (1993); Chothia and Lesk, J. Mol. 196, 1987, pp. 901). Another method uses a particular framework from the consensus sequence of all human antibodies of a particular subgroup of light or heavy chains. The same framework can be used for several different humanized antibodies (Carter et al., PNAS 89, pp. 4285 (1992); Presta et al., J. Immunol., 151, p. 2623 (1993)).

It is further important that antibodies be humanized with retention of high affinity for MICA receptors and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen (s), is achieved. In general, the CDR residues are directly and most substantially involved in influencing antigen binding. In a one example, the FRs of a humanized antibody chain are derived from a human variable region having at least about 60% overall sequence identity, and preferably at least about 80% overall sequence identity, with the variable region of the nonhuman donor (e.g., an 19E9 antibody). Optionally, the humanized heavy and/or light chain variable region shares at least about 60%, 70% or 80% overall sequence identity with the respective heavy and/or light chain variable region of the nonhuman donor (e.g., an 19E9 antibody).

Another method of making “humanized” monoclonal antibodies is to use a XenoMouse (Abgenix, Fremont, Calif.) as the mouse used for immunization. A XenoMouse is a murine host that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference. Human antibodies may also be produced according to various other techniques, such as by using, for immunization, other transgenic animals that have been engineered to express a human antibody repertoire (Jakobovitz et al., Nature 362 (1993) 255), or by selection of antibody repertoires using phage display methods. Such techniques are known to the skilled person and can be implemented starting from monoclonal antibodies as disclosed in the present application.

Antibody-Drug Conjugates

The antigen binding protein (e.g. antibody) molecule and non-antibody moiety can be connected by means of a linker. In such embodiments, the immunoconjugate is represented by Formula (I):

Ab-(X-(Z)ₙ)ₘ Formula (I)

wherein,

Ab is an anti-MICA antigen binding protein (e.g. an antibody or antibody fragment);

X is a moiety which connects Ab and Z, e.g., the residue of a linker following covalent linkage to one or both of Ab and Z;

Z is a cytotoxic agent;

n is an integer selected from 1 or 2; and

m is an integer selected from among 1 to 8, optionally m is an integer selected from among 1 to 6, optionally m is an integer selected from among 1 to 4, optionally m is 2 or 4.

In one embodiment, X represents a moiety that is cleavable, e.g., under physiological conditions, optionally under intracellular conditions.

The variable m represents the number of -X-Z moieties per antibody molecule in an immunoconjugate of formula (I). In various embodiments, m is 2 or 4. In some embodiments, m is 2 and n is 1. In some embodiments, m is 2 and n is 2. In some embodiments, m is 4 and n is 1. In some compositions comprising a plurality of immunoconjugates of formula (I), n is 1 and m is the average number of -X-(Z)ₙ moieties per Ab, in which case m can also be referred to as the average drug loading or drug:antibody ratio (DAR). Average drug loading or DAR may advantageously range from 2 to about 4 -X-(Z)ₙ moieties per Ab, wherein n is 1. In some embodiments, when m represents the average drug loading, m is about 2 (and n is 1), optionally wherein m is from about 1.6 to about 2.4, from about 1.8 to about 2.2, from about 1.6 to about 2, or from about 1.8 to about 2. In some embodiments, when m represents the average drug loading, m is about 4 (and n is 1), optionally wherein m is from about 3.6 to about 4.4, from about 3.8 to about 4.2, from about 3.6 to about 2, or from about 3.8 to about 2.

The number of -X-Z moieties per Ab may be characterized by conventional means such as mass spectroscopy, ELISA assay, and HPLC. The quantitative distribution of immunoconjugates in terms of m may also be determined. In some instances, separation, purification, and characterization of homogeneous immunoconjugates where m is a certain value, as distinguished from immunoconjugates with other drug loadings, may be achieved by means such as reverse phase HPLC or electrophoresis.

In some embodiments, e.g., as described in the Examples, the immunoconjugates of formula (I) exist as substantially homogenous compositions, wherein at least 80%, 90% or 95% of the immunoconjugates in a composition have the same m values (e.g., the same m value and the same n value); optionally n is 1 or 2 and m is 2 or 4; optionally m is 2 and n is 1 or 2; optionally m is 4 and n is 1.

A variety of methods can be used to covalently link a cytotoxic agent to an antibody, either non-specifically or specifically to a particular amino acid residue.

For example, different suitable linkers (e.g., heterobifunctional reagents for connecting an antibody molecule to a therapeutic agent or label) and methods for preparing immunoconjugates are known in the art. (See, for example, Chari et al, Cancer Research 52: 127-131 (1992)). The linker (e.g., X in formula I) can be cleavable, e.g., under physiological conditions, optionally as shown in the Examples under intracellular conditions, such that cleavage of the linker releases the cytotoxic agent in the intracellular environment.

The linker can be bonded to a chemically reactive group on the antibody moiety, e.g., to a free amino, imino, hydroxyl, thiol or carboxyl group (e.g., to the N- or C-terminus, to the epsilon amino group of one or more lysine residues, the free carboxylic acid group of one or more glutamic acid or aspartic acid residues, or to the sulfhydryl group of one or more cysteinyl residues). The site to which the linker is bound can be a natural residue in the amino acid sequence of the antibody moiety or it can be introduced into the antibody moiety, e.g., by DNA recombinant technology (e.g., by introducing a cysteine or protease cleavage site in the amino acid sequence) or by protein biochemistry (e.g., reduction, pH adjustment or proteolysis).

One of the most commonly used non-specific methods of covalent attachment is the carbodiimide reaction to link a carboxy (or amino) group of a compound to amino (or carboxy) groups of the antibody molecule. Additionally, bifunctional agents such as dialdehydes or imidoesters have been used to link the amino group of a compound to amino groups of an antibody molecule. Also available for attachment of drugs to antibody molecules is the Schiff base reaction. This method involves the periodate oxidation of a drug that contains glycol or hydroxy groups, thus forming an aldehyde which is then reacted with the antibody molecule. Attachment occurs via formation of a Schiff base with amino groups of the antibody molecule. Isothiocyanates can also be used as coupling agents for covalently attaching drugs to antibody molecule. Other techniques are known to the skilled artisan.

In certain embodiments, an intermediate, which is the precursor of the linker (X), is reacted with the cytotoxic agent (Z) under appropriate conditions. In certain embodiments, reactive groups are used on the cytotoxic agent and/or the intermediate. The product of the reaction between the cytotoxic agent and the intermediate, or the derivatized cytotoxic agent, is subsequently reacted with the antibody molecule under appropriate conditions.

In some embodiments, the linker (e.g., X in formula I) is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells. Most typical are peptidyl linkers that are cleavable by enzymes that are present in cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker). Other examples of such linkers are described, e.g., in U.S. Pat. No. 6,214,345, incorporated herein by reference in its entirety and for all purposes. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the val-cit linker). One advantage of using intracellular proteolytic release of the therapeutic agent is that the agent is typically attenuated when conjugated and the serum stabilities of the conjugates are typically high.

In other embodiments, the cleavable linker is pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene).

In yet other specific embodiments, the linker is a malonate linker (Johnson et al, 1995, Anticancer Res. 15: 1387-93), a maleimidobenzoyl linker (Lau et al. , 1995, BioorgMed Chem. 3(10): 1299-1304), or a 3′-N-amide analog (Lau et al, 1995, Bioorg. Med. Chem. 3(10):1305-12).

In some embodiments, a linker can function as a spacer to distance an antibody from an agent in order to avoid interference with binding capabilities. In one embodiment, a linker may comprise a stretcher unit, e.g., a molecule that forms a bond with a sulfur atom, a primary or secondary amino group or a carbohydrate group of the antibody, and which stretcher links the antibody to the cytotoxic agent (Z) or to an amino acid unit which is in turn linked to Z. When the stretcher is linked to an amino acid unit, the amino acid unit can be directly linked to Z or can comprise a spacer element (e.g., a non-self immolative or a self immolative spacer) linking the amino acid unit and Z. The amino acid unit can be a single amino acid or a peptide, e.g., valine-citrulline or phenyalanine-lysine.

In certain embodiments, site specific methods are used to conjugate a cytotoxic agent to an amino acid residue of the antibody. Various methods can be used to conjugate to a particular type of residue. Residues that can be conjugated to a cytotoxic agent according to such methods include any suitable natural amino acid residue, e.g. a cysteine, a lysine, a glutamine, or the asparagine at Kabat heavy chain position N297 (via the glycan bound to N297), as well as suitable unnatural amino acid residues, e.g., amino acids with bio-orthogonal reactive groups such as selenocysteine or acetylphenylalanine (pAcPhe),

In certain embodiments, site specific method, optionally enzyme catalyzed methods are used to conjugate a cytotoxic agent to an amino acid residue of the antibody.

In one embodiment, the antigen binding protein comprises an enzymatic recognition tag comprising an acceptor residue or sequence of amino acids comprising an acceptor residue that when incorporated into (e.g., appended to, inserted into) a polypeptide sequence of the antigen binding protein, under suitable conditions, is recognized by an enzyme and leads to cross-linking by the enzyme through a reaction between an amino acid (e.g. a side chain thereof) within the sequence of amino acids and a reaction partner (e.g. a linker that comprises the cytotoxic agent or a reactive group that is then used to further linker to a cytotoxic reagent in a further reactive step). The recognition tag may be a peptide sequence that is not naturally present in the polypeptide comprising the enzyme recognition tag.

For example, the formylglycine generating-enzyme (FGE) recognizes and acts specifically on a pentapeptide (CXPXR), oxidizing the cysteine to an unusual aldehyde bearing formylglycine residue (fGly); it recognizes a consensus amino acid sequence, termed the sulfatase motif, and converts a Cys in the consensus sequence to an FGly through a novel oxidation process. The broadly defined minimal consensus motif recognized by FGE is the pentamer CxPxR, where x primarily consists of a neutral amino acid residue. High conversion of Cys to FGly is possible when this small FGE target sequence, termed the aldehyde tag, is inserted into heterologous protein sequences and expressed using standard recombinant expression methods. The aldehyde generated by FGE on the target protein can be selectively reacted with alpha-nucleophiles, such as aminooxy- and hydrazide-bearing compounds, generating oxime- and hydrazone-ligated products, respectively.

In one example, a sortase enzyme is employed to catalyze the functionalization of acceptor amino acid residues on antibodies, e.g., within a sortase recognition tag peptide engineered within or appended to a constant region of the antibody. See, e.g., WO2013/155526 and WO2013/003555.

In another example, a transglutaminase (TGase) enzyme is employed to catalyze the stoichiometrically functionalization of acceptor glutamines on antibodies with a cytotoxic agent. For example, TGase-catalyzed methods can be used to functionalize antibodies with large and/or hydrophobic substrates (for example pyrrolobenzodiazepine dimers which represent highly hydrophobic organic molecules). Coupling of cytotoxic moieties to acceptor glutamines on antibody constant regions using TGase-catalyzed methods, and linkers suitable therefore, are described in PCT publication nos. WO2013/309283 and WO2014/202775 (Innate Pharma), the disclosures of which are incorporated herein by reference. Using this approach one can advantageously conjugate the glutamine naturally present at Kabat residue 295 and/or at a glutamine introduced at residue 297 via the substitution of the asparagine by a glutamine.

In another example, a enzymatic method using endoglycosidase can be used to first trim the glycan tree of the glycan at Kabat residue N297, leaving only the core N-acetylglucosamine (GlcNAc) moiety (the so-called GlcNAc-protein), followed by a strategy for functionalization of the glycan with a reactive group such an azide or other group that can be catalytically reacted in a “click chemistry” cycloaddition reaction with a linker comprising a complementary reactive group and the cytotoxic agent (Z). See, e.g., WO2014/065661. The resulting immunoconjugate will have a functionalized asparagine residue at Kabat position 297.

The site specific enzyme-based methods are thus particularly useful not only to conjugate a linker bearing a cytotoxic agent (Z) to an antibody, but also for use in two-step methods, wherein a reactive group or “handle” is installed on an antibody, followed by a step of reacting the antibody with a linker comprising a complementary reactive group and the cytotoxic agent (Z).

As an exemplary method, TGase have been used to illustrate the immunoconjugates. Enzymes of the TG-family catalyze covalent protein crosslinking by forming proteinase resistant isopeptide bonds between a lysine donor residue of one protein and an acceptor glutamine residue of another protein, and is accompanied by the release of ammonia. The catalytic mechanism of transglutaminases has been proposed as follows. After the Glycine-containing first substrate (an acceptor glutamine) binds to the enzyme, it forms a γ-glutamylthioester with the cysteine residue in the active center of TGase, known as the acylenzyme intermediate, accompanied by the release of ammonia. The second substrate (donor or K-substrate) then binds to the acylenzyme intermediate and attacks the thioester bond. The product (two proteins crosslinked by an Nε(γ-glutamyl)lysine isopetide bridge) is formed and released. This re-establishes the active-centre Cys residue of the enzyme in its original form and allows it to participate in another cycle of catalysis. The formation of the covalent acylenzyme intermediate is thought to be the rate-limiting step in these reactions. The catalytic triad of many transglutaminases is papain-like, containing Cys-His-Asp (where His is histidine and Asp is aspartic acid) and, crucially, a tryptophan (Trp) residue located 36 residues away from the active-centre Cys. In contrast, bacterial TG isolated from Streptoverticillium sp (vide supra) has an atypical catalytic triad and shows no sequence homology with the papain-like catalytic triad of other TGases.

TGases display strict specificity in recognition of glutamine protein substrates. However, TGases display broad specificity for recognition of the acyl-acceptor amine group, which can either be the ε-amino group of peptidyl lysine or a low-molecular mass primary amine (frequently a polyamine) (see, e.g., Folk, et al. (1980) J. Biol. Chem. 255, 3695-3700). Thus a wide range of lysine-based linkers can be envisaged. For example, in addition to lysine, the small lysine-mimicking primary amine 5-pentylamine (cadaverin) and variants or fragments thereof can efficiently bind to the acylenzyme intermediate, and a pseudo-isopeptide bond with the glutamine-containing protein is formed. See, e.g., Lorand, L. et al. (1979) Biochemistry 18, 1756-1765 (1979); Murthy, S. N. et al. (1994). J. Biol. Chem. 269, 22907-22911 (1994); Murthy, P. et al. (2009) Biochemistry (2009).

Bacterial, archaeal and eukaryotic TGases have been characterized and differ in several ways from mammalian TGases (Lorand, L. & Graham, R. M. (2003) Nat. Rev. Mol. Cell Biol. 4, 140-156). BTG and more generally microbial TGases (EC 2.3.2.13, protein-glutamine-γ-glutamyltransferase) such as Streptomyces mobaraensis are calcium-independent and have an amino acid sequence of) very different from those of mammalian TGs (Ando et al. (1989) Agric. Biol. Chem. 53, 2613-2617). BTG is furthermore much smaller (37.8 kDa versus 76.6 kDa for guinea pig liver TG). Additionally, BTG shows broader substrate specificity for the amine acceptor glutamine substrates in proteins than do mammalian TGases. These characteristics, together with a higher reaction rate, low cost of production, and a decreased tendency to catalyze deamidation make BTG a preferred enzyme for use herein.

An acceptor glutamine present on an antibody (e.g., part of the antibody's primary structure, including for example an antibody or antibody fragment with a peptide tag) will, under suitable conditions, be recognized by a TGase and covalently bound to a lysine-based linker. Resulting antibody conjugates can be analyzed using any suitable method. Preferably, the stoichiometry of the conjugated antibodies can be characterized by liquid chromatography mass spectrometry (LC/MS) using a top-down approach in order to assess the number of lysine-based linker and/or where applicable cytotoxic agent moieties conjugated to antibodies, and in particular the homogeneity of the composition. Conjugates can be reduced before LC/MS analysis and light chains and heavy chains are measured separately. Examples of glutamine-bearing peptide tags recognized by TGase that can be fused to and/or inserted into a light chain or heavy chain constant region include, e.g. those disclosed in WO2012/059882, WO2015/097267 and WO2014/072482, the disclosures of which, including any glutamine-containing peptide tags and motifs, are incorporated by reference. In other examples, peptide tags comprising a lysine are also known be recognized by a TGase and covalently bound to a glutamine-based linker. Such peptide tags can be placed within or appended to heavy or light chain constant region, e.g. positioned C-terminal of the C-terminus of the heavy and/or light chain of an antibody or antibody fragment, or optionally positioned within an Fc domain.

If the acceptor glutamine is the glutamine residue naturally present at residue Q295 (Kabat EU numbering) and/or a glutamine introduced by amino acid substitution at nearby residues such as residue N297, the antibodies that are to be conjugated to the lysine-based linker will be free of N-linked glycosylation at residue N297 (Kabat EU numbering). Full-length wild-type IgG antibodies may be prepared such that they lack N297-linked glycosylation, thereby permitting TGase-mediated conjugation onto glutamine residue Q295 (and/or at residue 297 if the antibody bears a N297Q substitution) naturally present in the CH2 domain. Enzymatic deglycosylation can be carried out as described herein or according to any suitable method. For example, antibody (1 mg) in PBS buffer (0.1 mol/L NaCl and 0.05 mol/L sodium phosphate buffer, pH 7.4) are incubated with 100 units (0.2 μL) of N-glycosidase F (PNGase F) from Flavobacterium meningosepticum (New England BioLabs, Ipswich, UK) at 37° C. overnight. The enzyme is then removed by centrifugation-dialysis (Vivaspin MWCO 50 kDa, Vivascience, Winkel, Switzerland). The product can be analyzed by LC/MS. Alternatively, antibody Fc domains can be engineered to introduce an amino acid substation that prevents N-linked glycosylation. For example a N297X substitution can be introduced into a heavy chain of an antibody, wherein X is any amino acid other than asparagine. When X is a glutamine residue, the glutamine at residue 297 will serve as an acceptor glutamine and will be coupled to a linker by TGase.

Antibodies can be engineered to have the desired number of acceptor glutamines. In one example, an antibody comprises a single acceptor glutamine at Kabat residue 295 on each heavy chain (e.g., the antibody comprises a N297X substitution, where X is any amino acid other than asparagine or glutamine, or the antibody is enzymatically deglycosylated); the antibody will have a total of two acceptor glutamines. In one example, an antibody comprises a single acceptor glutamine at residue 297 of each heavy chain (e.g., the antibody comprises a Q295X substitution, where X is any amino acid other than glutamine, and a N297Q substitution); the antibody will have a total of two acceptor glutamines. In one example, an antibody comprises an acceptor glutamine at residues 295 and 297 of each heavy chain (e.g., the antibody comprises a N297Q substitution); the antibody will have a total of four acceptor glutamines. In one example, an antibody comprises one or more acceptor glutamines within a TGase recognition tag fused, optionally via intervening amino acid residues, to a C-terminus of a light and/or heavy chain.

In one embodiment, the product is analyzed for drug loading (e.g., number of conjugates per antibody or DAR). Such methods can be used to determine the mean number of conjugates per antibody (e.g., the mean DAR) as well as the distribution of number of conjugates per antibody in a composition, i.e., the percentage of total antibody with any given level of drug loading or DAR. The portion of antibodies having a number (n) of conjugated acceptor glutamines (e.g., n=1, 2, 3 or 4) can be determined. One technique adapted to such determination and more generally drug loading is hydrophobic interaction chromatography (HIC), HIC can be carried out as described for example in Hamblett et al. (2004) Cancer Res. 10: 7063-7070; Wakankar et al. (2011) mAbs 3(2): 161-172; and Lyon et al (2012) Methods in Enzymology, Vol. 502: 123-138, the disclosure of which are incorporated herein by reference.

Examples of useful TGases include microbial transglutaminases, such as e.g., from Streptomyces mobaraense, Streptomyces cinnamoneum and Streptomyces griseocarneum (for discussion of suitable TGases, see, e.g., PCT publication nos. WO2013/309283 and WO2014/202775). A preferred TGase is bacterial transglutaminase (BTG) (see, e.g., EC 2.3.2.13, protein-glutamine-γ-glutamyltransferase). In a more preferred embodiment, the TGase is from S. mobaraense. In another embodiment, the TGase is a mutant TGase having at least 80% sequence homology with native TGase. A preferred example is recombinant bacterial transglutaminase derived from streptomyces mobaraensis (available from Zedira, Darmstadt, Germany).

The TGase-catalyzed reaction can be carried out under mild conditions, from several hours to a day (e.g., overnight). Recombinant BTG (EC 2.3.2.13) from streptomyces mobaraensis (Zedira, Darmstadt, Germany) can be used at a concentration of between 1 and 20 U/mL, preferably between 6 U/mL and 20 U/mL. The lysine-based linker substrates are reacted with antibody (1 mg/mL) at ligand concentrations between 400 and 600 mol/L, providing a 60 to 90-fold excess of the substrates over the antibody, or optionally at lower excess of substrates, e.g., 1- to 20-fold, or 10-20 fold. The reactions are performed in potassium-free phosphate buffered saline (PBS; pH 8) at 37° C. After 4 h to several days (depending on the antibody and the ligand), steady-state conditions are achieved. Excess ligand and enzyme are then removed using centrifugation-dialysis (Vivaspin MWCO 50 kDa, Vivascience, Winkel, Switzerland). Reactions are monitored by LC/MS. Higher amounts of TGase can be used as a function of different lysine-derivatives and substrates.

In one aspect, provided is a method for conjugating a cytotoxic agent (Z) to an antibody, comprising the steps of:

a) providing an antibody of the disclosure, wherein the antibody comprises at least one acceptor glutamine residue in or appended to a heavy and/or light chain constant region; and

b) reacting said antibody with a linker comprising a primary amine (a lysine-based linker) comprising a cytotoxic agent (Z), in the presence of a TGase, under conditions sufficient to obtain an antibody comprising an acceptor glutamine linked (covalently) to a cytotoxic agent (Z) via said linker.

Certain aspects of the disclosure are directed to a linking reagent that can be attached, by the action of a TGase, to a polypeptide at a glutamine residue (Q) within the sequence of the antibody (Ab). The linking reagent comprises a lysine derivative (Lys) or a functional equivalent thereof, that is connected to at least one reactive group (R) or a cytotoxic agent (Z). The lysine derivative (Lys) or a functional equivalent can comprise generally any primary amine chain which is a substrate for TGase, e.g., comprising an alkylamine, oxoamine. The reactive group is preferably a functionality that is insensitive to water but selectively undergoes a very high conversion addition reaction with a complementary reagent. The functional equivalent of a lysine derivative may comprise a 2 to 20 carbon chain, or a functional equivalent thereof, with an H₂N or H₂NCH₂ (aminomethylene) group, or a protected H₂N or H₂NCH₂ group that can be derived from the H₂N or aminomethylene positioned at one or more ends of the carbon chain. The functional equivalent of the carbon chain may comprise a chain of 3 to 20 atoms where one or more of the atoms other than the primary amine can be other than carbon, for example oxygen, sulfur, nitrogen, or other atoms, e.g., with an H₂NOCH₂ group, or a protected H₂NOCH₂ group positioned at one or more ends of the carbon chain. The oxygen, sulfur, or nitrogen atom can be of an ether, ester, thioether, thioester, amino, alkylamino, amido or alkylamido functionality within the carbon chain. Suitable linkers are described, for example, in PCT publication nos. WO2013/309283 and WO2014/202775.

One exemplary functional equivalent of the carbon chain is an oligo (ethylene oxide) chain. The functionality within the carbon chain can be included to couple the reactive group to the H₂N H₂NOCH₂ or H₂NCH₂ group or protected H₂N, H₂NOCH₂ or H₂NCH₂ group. The carbon chain, or its functional equivalent, can be substituted or unsubstituted. For example, the carbon chain, or its functional equivalent can comprise a plurality of (CH₂— CH₂—O—) groups, optionally (CH₂— CH₂—O—)ₙ group wherein n is an integer selected among the range of 1 to 6. The substituents can be alkyl groups, aryl groups, alkyl aryl groups, carboxylic acid groups, amide groups, hydroxy groups, or any other groups that do not compete with the amino group for, or inhibit, conjugation with a glutamine residue of the protein. Typically, when a substituent is present, its presence is in a convenient starting material, such as the carboxylic acid group of lysine, from which the lysine derivative results. The amine at the end of a carbon chain or functional equivalent is necessarily included in the linking reagent.

Examples of starting materials for the functional equivalent of lysine can be an α,ω-diaminoalkane, for example, 1,2-diaminoethane, 1,3-diaminopropane, 1,4-diaminobutane, 1,5-diaminopentane, 1,6-diaminohexane, 1,7-diaminoheptane, 1,8-diaminooctane, 1,9-diaminononane, 1,10-diaminodecane, 1,11-diaminoundecane, or 1,12-diaminododecane. Other starting materials for the functional equivalent of a lysine derivative can be α,ω-diamino oligo (ethylene oxide), for example, H₂N(CH₂CH₂O)ₓCH₂CH₂NH₂ where x is an integer selected among the range of 1 to 6. The α,ω-diamino oligo (ethylene oxide) can be a single oligomer or it can be a mixture of oligomers where x defines an average size. An exemplary protected H₂NCH₂ is the tert-butylcarbamate protected amine of tert-butyl N-(5-aminopentyl)carbamate (N-Boc-cadaverin).

Linking reagents used for direct (one-step) linking of a cytotoxic agent (Z) to an antibody will advantageously comprise an element that functions as a spacer to distance a large, charged or hydrophobic organic cytotoxic agent (Z) from the acceptor glutamine. The spacer may be embodied in the lysine derivative or functional equivalent thereof, or in a further element of the linker (e.g., an L, V and/or Y group as further described herein). In one embodiment, the element that functions as a spacer is a lysine derivative (Lys) or a functional equivalent thereof having a structure NH—(C)—, wherein (C) is a substituted or unsubstituted alkyl or heteroalkyl chain, wherein any carbon of the chain is optionally substituted with an alkoxy, hydroxyl, alkylcarbonyloxy, alkyl-S—, thiol, alkyl-C(O)S—, amine, alkylamine, amide, or alkylamide, and where the chain length is an integer greater than 10 atoms, an integer from among the range of 10 to 20. For example, C can be a carbon comprising framework of 10 to 20 atoms substituted at one or more atoms. In one embodiment, the linking reagent comprises an L, V and/or Y group that functions as a spacer and is positioned between the NH—(C)— group and the cytotoxic agent (Z), wherein L is a carbon-comprising framework of 1 to 200 atoms substituted at one or more atoms, optionally wherein the carbon comprising framework is a linear hydrocarbon, a symmetrically or asymmetrically branched hydrocarbon, monosaccharide, a glycan, disaccharide, linear or branched oligosaccharide (asymmetrically branched or symmetrically branched), other natural linear or branched oligomers (asymmetrically branched or symmetrically branched), amino acid residue, di-, tri- or oligopeptide, or any dimer, trimer, or higher oligomer (linear, asymmetrically branched or symmetrically branched) for example resulting from any chain-growth or step-growth polymerization process; V is a non-cleavable moiety or a conditionally-cleavable moiety, optionally following prior conditional transformation, which can be cleaved or transformed by a chemical, photochemical, physical, biological, or enzymatic process (e.g., cleavage of V ultimately leading to release of one or more moieties subsequently or ultimately linked to V, for example a Z moiety). In some embodiments, V is, preferably, a di-, tri-, tetra-, or oligopeptide (e.g., a valine-citrulline containing peptide, or the like) and Y is a spacer system (e.g., a self-eliminating spacer system or a non-self-elimination spacer system) which is comprised of 1 or more spacers. The spacer system Y may self-eliminating or non-self-eliminating. A “self-eliminating” spacer unit allows for release of the drug moiety without a separate hydrolysis step. When a self-eliminating spacer is used, after cleavage or transformation of V, the side of Y linked to V becomes unblocked, which results in eventual release of one or more moieties Z. Y may for example be any straight, branched and/or cyclic C₂₋₃₀ alkyl, C₂₋₃₀ alkenyl, C₂₋₃₀ alkynyl, C₂₋₃₀ heteroalkyl, C₂₋₃₀ heteroalkenyl, C₂₋₃₀ heteroalkynyl, optionally wherein one or more homocyclic aromatic compound radical or heterocyclic compound radical may be inserted; notably, any straight or branched C₂₋₅ alkyl, C₅₋₁₀ alkyl, C₁₁₋₂₀ alkyl, —O—C₁₋₅ alkyl, —O— C₅₋₁₀ alkyl, —O— C₁₁₋₂₀ alkyl, or (CH₂— CH₂—O—)₁₋₂₄ or (CH₂)ₓ₁—(CH₂—O—CH₂)₁₋₂₄—(CH₂)ₓ₂— group, wherein x1 and x2 are independently an integer selected among the range of 0 to 20, an amino acid, an oligopeptide, glycan, sulfate, phosphate, or carboxylate. Optionally, Y is absent. In some embodiments, Y is a C₂₋₆ alkyl group. The self-elimination spacer systems may for example be those described in WO 02/083180 and WO 2004/043493, which are incorporated herein by reference in their entirety, as well as other self-elimination spacers known to a person skilled in the art. In certain embodiments, a spacer unit of a linker comprises a p-aminobenzyl unit. In one such embodiment, a p-aminobenzyl alcohol is attached to an amino acid unit via an amide bond, and a carbamate, methylcarbamate, or carbonate is made between the benzyl alcohol and a cytotoxic agent. In one embodiment, the spacer unit is p-aminobenzyloxycarbonyl (PAB). Examples of self-eliminating spacer units further include, but are not limited to, aromatic compounds that are electronically similar to p-aminobenzyl alcohol (see, e.g., US 2005/0256030 A1), such as 2-aminoimidazol-5-methanoi derivatives (Hay et al. (1999) Bioorg. Med. Chem. Lett. 9:2237) and ortho- or para-aminobenzylacetals. Spacers can be used mat undergo cyclization upon amide bond hydrolysis, such as substituted and unsubstituted 4-aminobutyric acid amides (Rodrigues et al. Chemistry Biology, 1995, 2, 223) and 2-aminophenylpropionic acid amides (Amsberry, et al., J. Org. Chem., 1990, 55. 5867). Elimination of amine-containing drugs that are substituted at the a-position of glycine (Kingsbury, et al., J. Med. Chem., 1984, 27, 1447) are also examples of self-immolative spacers.

V may comprise for example a carbon comprising framework of 1 to 200 atoms, optionally a carbon comprising framework of at least 10 atoms, e.g., 10 to 100 atoms or 20 to 100 atoms, substituted at one or more atoms, optionally wherein the carbon comprising framework is a linear hydrocarbon or comprises a cyclic group, a symmetrically or asymmetrically branched hydrocarbon, monosaccharide, disaccharide, linear or branched oligosaccharide (asymmetrically branched or symmetrically branched), other natural linear or branched oligomers (asymmetrically branched or symmetrically branched), an amino acid, a di-, tri-, tetra-, or oligopeptide, or more generally any dimer, trimer, or higher oligomer (linear, asymmetrically branched or symmetrically branched) resulting from any chain-growth or step-growth polymerization process. V may for example be any straight, branched and/or cyclic C₂₋₃₀ alkyl, C₂₋₃₀ alkenyl, C₂₋₃₀ alkynyl, C₂₋₃₀ heteroalkyl, C₂₋₃₀ heteroalkenyl, C₂₋₃₀ heteroalkynyl, optionally wherein one or more homocyclic aromatic compound radical or heterocyclic compound radical may be inserted; notably, any straight or branched C₂₋₆ alkyl, C₅₋₁₀ alkyl, C₁₁₋₂₀ alkyl, —O— C₁₋₅ alkyl, —O— C₅₋₁₀ alkyl, —O— C₁₁₋₂₀ alkyl, or (CH₂— CH₂—O—)₁₋₂₄ or (CH₂)ₓ₁—(CH₂—O—CH₂)₁₋₂₄—(CH₂)ₓ₂-group, wherein x1 and x2 are independently an integer selected among the range of 0 to 20, an amino acid, an oligopeptide, glycan, sulfate, phosphate, or carboxylate. Optionally, V may be or absent. In some embodiments, V is a C₂-6 alkyl group.

In certain embodiment, V contains a di-, tri-, tetra-, or oligopeptide which consists of an amino acid sequence recognized by a protease. The tripeptide may be linked via its C-terminus to Y. In one embodiment, the C-terminal amino acid residue of the tripeptide is selected from arginine, citrulline, and lysine, the middle amino acid residue of the tripeptide is selected from alanine, valine, leucine, isoleucine, methionine, phenylalanine, cyclohexylglycine, tryptophan and proline, and the N-terminal ammo acid residue of the tripeptide is selected from any natural or unnatural amino acid. In one embodiment the disclosure provides to a compound wherein V comprises a dipeptide. The dipeptide may be linked via its C-terminus to Y. In one embodiment, the C-terminal amino acid residue of the dipeptide is selected from alanine, arginine, citrulline, and lysine, and the N-terminal amino acid residue of the dipeptide is selected from any natural or unnatural amino acid. In one embodiment, V is selected from phenylalanine-lysine and valine-citrulline.

In one embodiment provided is an antibody or antibody fragment as described in any embodiment herein, wherein the antibody comprises a functionalized acceptor glutamine residue, optionally wherein said acceptor glutamine residue is flanked at the +2 position by a non-aspartic acid residue, the functionalized acceptor glutamine residue having Formula II,

(Q)-N H—C-X-L-(V-(Y-(Z)_{z})_{q})ᵣ Formula II

or a pharmaceutically acceptable salt or solvate thereof,

wherein:

Q is a glutamine residue present in an antibody or antibody fragment;

C is a substituted or unsubstituted alkyl or heteroalkyl chain, optionally wherein any carbon of the chain is substituted with an alkoxy, hydroxyl, alkylcarbonyloxy, alkyl-S—, thiol, alkyl-C(O)S—, amine, alkylamine, amide, or alkylamide;

X is NH, O, S, absent, or a bond;

L is independently absent, a bond or a continuation of a bond, or a carbon comprising framework of 5 to 200 atoms substituted at one or more atoms, optionally, wherein the carbon comprising framework comprises a linear framework of 5 to 30 carbon atoms optionally substituted at one or more atoms, optionally wherein the carbon comprising framework is a linear hydrocarbon, a symmetrically or asymmetrically branched hydrocarbon, monosaccharide, disaccharide, linear or branched oligosaccharide (asymmetrically branched or symmetrically branched), other natural linear or branched oligomers (asymmetrically branched or symmetrically branched), or a dimer, trimer, or higher oligomer (linear, asymmetrically branched or symmetrically branched) resulting from any chain-growth or step-growth polymerization process;

r is an integer selected from among 1, 2, 3 or 4;

q is an integer selected from among 1, 2, 3 or 4;

z is an integer selected from among 1, 2, 3 or 4; and

V is independently absent, a non-cleavable moiety or a conditionally-cleavable moiety;

Y is independently absent, a bond or a continuation of a bond, or a spacer system which is comprised of 1 or more spacers; and

Z is a cytotoxic agent.

In one embodiment, r, q and z are each 1. In one embodiment, one of r, q and z is 2, and the other of r, q and z are each 1.

In one embodiment of such method, the Z comprises a cytotoxic agent (e.g. a DNA minor groove binding agent) that is water soluble.

In some embodiments, particularly when highly hydrophobic cytotoxic agents are conjugated to antibodies, organic solvent is generally required to maintain solubility and avoid formation of aggregated antibody-drug conjugates. Where a hydrophobic linker substrate cannot be solubilized without high concentrations of organic solvent (e.g., linkers comprising pyrrolobenzodiazepine moieties require as much as 50% solvent to permit highly homogenous coupling at a DAR of 2), a multi-step coupling process making use of a linker with a reactive moiety can be used to achieve highly homogenous compositions. Organic solvent at 5% or higher, however, inhibits the ability of TGase to conjugate efficiently, resulting in incomplete coupling (less than 90% or acceptor glutamines functionalized) when the linker with reactive moiety is coupled to an antibody, e.g., at residue Q295 and/or at residue 297 when the antibody comprises a N297Q mutation. Consequently, the coupling can be carried out by reacting an antibody comprising an acceptor glutamine residue with a linking reagent with reactive moiety (R), in the presence of a transglutaminase enzyme, wherein solvent (e.g., organic solvent, polar solvent, non-polar solvent, DMSO) is absent or is present at less than 10% (v/v), optionally further less than 5%, 4%, 3% or 2% (v/v). The resulting antibody is then reacted with a linker comprising a complementary reactive group (R′) and a hydrophobic drug (e.g., a pyrrolobenzodiazepine moiety) to yield an antibody coupled (e.g., via the reaction produce of R and R′) to the hydrophobic drug (Z). The reaction step with linker comprising a complementary reactive group (R′) and a hydrophobic drug can be carried out in the presence of solvent, e.g., wherein solvent (e.g., organic solvent, polar solvent, non-polar solvent, DMSO) is present in the reaction mixture, wherein solvent is present is present at more than 2%, 3%, 4%, 5%, 10%, 20%, 40% or 50% (v/v). In one embodiment of such method, the Z comprises a DNA minor groove binding agent that requires organic solvent for solubility (e.g. a DNA minor groove binding agent that is not water soluble).

The R and complementary R′ reactive groups can each be any suitable reactive moiety, for example a moiety comprising an unprotected or protected bioorthogonal-reaction compatible reactive group, for example an unprotected or protected thiol, epoxide, maleimide, haloacetamide, o-phoshenearomatic ester, azide, fulminate, sulfonate ester, alkyne, cyanide, amino-thiol, carbonyl, aldehyde, generally any group capable of oxime and hydrazine formation, 1,2,4,5-tetrazine, norbornene, other stained or otherwise electronically activated alkene, a substituted or unsubstituted cycloalkyne, generally any reactive groups which form via bioorthogonal cycloaddition reaction a 1,3- or 1,5-disubstituted triazole, any diene or strained alkene dienophile that can react via inverse electron demand Diels-Alder reaction, a protected or unprotected amine, a carboxylic acid, an aldehyde, or an oxyamine.

The reactive groups can for example chosen to undergo thio-maleimide (or haloacetamide) addition, Staudinger ligation, Huisgen 1,3-cycloaddition (click reaction), or Diels-Alder cycloaddition with a complementary reactive group attached to an agent comprising a therapeutic moiety, a diagnostic moiety, or any other moiety for a desired function. In one embodiment, the reactive group is a haloacetamide, (e.g., bromo-acetamide, iodo-acetamide, chloroacetamide). Such reactive groups will be more stable in vivo (and in serum) compared with maleimide groups.

In one advantageous embodiment, the reactive groups R and R′ are complementary reagents capable of undergoing a “click” reaction. For example a 1,3-dipole-functional compound can react with an alkyne in a cyclization reaction to form a heterocyclic compound, preferably in the substantial absence of added catalyst (e.g., Cu(I)). A variety compounds having at least one 1,3-dipole group attached thereto (having a three-atom pi-electron system containing 4 electrons delocalized over the three atoms) can be used to react with the alkynes disclosed herein. Exemplary 1,3-dipole groups include, but are not limited to, azides, nitrile oxides, nitrones, azoxy groups, and acyl diazo groups. Examples include o-phosphenearomatic ester, an azide, a fulminate, an alkyne (including any strained cycloalkyne), a cyanide, an anthracene, a 1,2,4,5-tetrazine, or a norbornene (or other strained cycloalkene).

In one aspect, a cytotoxic agent (Z) (e.g., a hydrophobic, high molecular weight and/or charged organic compound) can be conjugated to an anti-MICA antibody of the disclosure using a method comprising the steps of:

a) providing an anti-MICA antibody of the disclosure comprising at least one acceptor glutamine residue; and

b) reacting said antibody with a linker comprising a primary amine (a lysine-based linker) comprising a reactive group (R), in the presence of a TGase, under conditions sufficient to obtain an antibody comprising an acceptor glutamine linked (covalently) to a reactive group (R) via said linker, wherein the reaction mixture is free of organic solvent or contains less than 10% (v/v) or contains less than 5%, 4%, 3% or 2% (v/v) organic solvent; and

c) reacting, optionally in the presence of organic solvent (e.g., at least 2%, 3%, 4%, 5%, 10%, 20%, 25%, 40% or 50% (v/v) organic solvent):
(i) an antibody of step (b) comprising an acceptor glutamine linked to a reactive group (R) via the linker (the lysine-based linker), with
(ii) a compound comprising a cytotoxic agent (Z) and a reactive group (R′) capable of reacting with reactive group R,

under conditions sufficient to obtain an antibody comprising an acceptor glutamine linked to the cytotoxic agent (Z) via a linker comprising a primary amine (a lysine-based linker). In one embodiment of such method, the Z comprises a DNA minor groove binding agent that requires organic solvent for solubility (e.g. a DNA minor groove binding agent that is not water soluble). The resulting antibody can be characterized by the structure of formula III. In one embodiment provided is an antibody or antibody fragment according to any embodiment herein, wherein the antibody comprises a functionalized acceptor glutamine residue, optionally wherein said acceptor glutamine residue is flanked at the +2 position by a non-aspartic acid residue, the functionalized acceptor glutamine residue having the structure of Formula III,

(Q)-NH—C-X-L-(V-(Y-(M)_{z})_{q})ᵣ Formula III

or a pharmaceutically acceptable salt or solvate thereof,

wherein:

Q is a glutamine residue present in an antibody or antibody fragment;

C is a substituted or unsubstituted alkyl or heteroalkyl chain, optionally wherein any carbon of the chain is substituted with a alkoxy, hydroxyl, alkylcarbonyloxy, alkyl-S—, thiol, alkyl-C(O)S—, amine, alkylamine, amide, or alkylamide;

X is NH, O, S, absent, or a bond;

L is independently absent, a bond or a continuation of a bond, or a carbon comprising framework of 1 to 200 atoms substituted at one or more atoms, optionally, wherein the carbon comprising framework comprises a linear framework of 3 to 30 carbon atoms optionally substituted at one or more atoms, optionally wherein the carbon comprising framework is a linear hydrocarbon, a symmetrically or asymmetrically branched hydrocarbon, monosaccharide, disaccharide, linear or branched oligosaccharide (asymmetrically branched or symmetrically branched), other natural linear or branched oligomers (asymmetrically branched or symmetrically branched), or a dimer, trimer, or higher oligomer (linear, asymmetrically branched or symmetrically branched) resulting from any chain-growth or step-growth polymerization process;

r is an integer selected from among 1, 2, 3 or 4;

q is an integer selected from among 1, 2, 3 or 4;

z is an integer selected from among 1, 2, 3 or 4;

V is independently absent, a bond or a continuation of a bond, a non-cleavable moiety or a conditionally-cleavable moiety;

Y is independently absent, a bond or a continuation of a bond, or a spacer system which is comprised of 1 or more spacers;

M is independently: R or (RR′)-L′-(V′-(Y′-(Z)_{z′})_{q′})_{r′}, wherein

R is a reactive moiety;

(RR′) is an addition product between R and a complementary reactive moiety R′;

L′ is independently absent, a bond or a continuation of a bond, or a carbon comprising framework of 1 to 200 atoms substituted at one or more atoms, optionally, wherein the carbon comprising framework comprises a linear framework of 3 to 30 carbon atoms optionally substituted at one or more atoms, optionally wherein the carbon comprising framework is a linear hydrocarbon, a symmetrically or asymmetrically branched hydrocarbon, monosaccharide, disaccharide, linear or branched oligosaccharide (asymmetrically branched or symmetrically branched), other natural linear or branched oligomers (asymmetrically branched or symmetrically branched), or a dimer, trimer, or higher oligomer (linear, asymmetrically branched or symmetrically branched) resulting from any chain-growth or step-growth polymerization process;

V′ is independently absent, a bond or a continuation of a bond, a non-cleavable moiety or a conditionally-cleavable moiety;

Y′ is independently absent, a bond or a continuation of a bond, or a spacer system which is comprised of 1 or more spacers;

Z is a cytotoxic agent, and each Z is directly coupled to either Y or V when Y is absent, or L when both Y and V are absent; and

z′, q′ and r′ are each independently an integer selected from among 1, 2, 3 or 4. In one embodiment, r, r′, q, q′, z and z′ are each 1. In one embodiment, one of r, r′, q, q′, z and z′ is 2, and the other of r, r′, q, q′, z and z′ are each 1.

In one embodiment, R and R′ are moieties capable of undergoing a Huisgen 1,3-cycloaddition reaction or an inverse electron demand Diels-Alder cycloaddition reaction, wherein one of R and R′ is an azide or a tetrazine and the other is a strained alkene or strained alkyne. Optionally, one of R and R′ is an azide and the other is a cyclooctyne. Optionally, one of R and R′ is a tetrazine and the other is a trans-cyclooctene (TCO).

In one embodiment, V is absent, a bond or a continuation of a bond and V′ is a non-cleavable moiety or a conditionally-cleavable moiety. In one embodiment, Y is absent, a bond or a continuation of a bond and Y′ is a spacer system which is comprised of 1 or more spacers.

The immunoconjugate can be purified from reactants by employing methodologies well known to those of skill in the art, e.g., column chromatography (e.g., affinity chromatography, ion exchange chromatography, gel filtration, hydrophobic interaction chromatography), dialysis, diafiltration or precipitation. The immunoconjugate can be evaluated by employing methodologies well known to those skilled in the art, e.g., SDS-PAGE, mass spectroscopy, or capillary electrophoresis.

In one embodiment, Z is or comprises a DNA minor groove binding agent, optionally a water soluble DNA minor groove binding agent, optionally a non-water soluble DNA minor groove binding agent, e.g. a DNA minor groove binding agent having high hydrophobicity, a DNA minor groove binding agent requiring organic solvent, e.g. at least 10%, 20%, 30% or more (v/v) organic solvent for solubility. In another embodiment, Z is a spliceosome inhibitor, for example a thailanstatin compound or an analog thereof (see, e.g., Nicolaou et al., (2016) J Am Chem Soc. 138(24):7532 Puthenveetil et al., (2016) Bioconjugate Chem. 27 (8), pp 1880-1888). Examples of DNA minor groove binding agents are anthracyclines, calicheamicins, duocarmycins, and pyrrolobenzodiazepines (PBDs). All of these drugs function by binding the minor groove of DNA and causing DNA stand scission, alkylation, or cross-linking. A variety of types of such agents are known in the art. In one embodiment, the Z moiety comprises a pyrrolobenzodiazepine. In one embodiment, Z is a pyrrolobenzodiazepine monomer, a pyrrolobenzodiazepine dimer or a pyrrolobenzodiazepine trimer. In one embodiment, Z is a pyrrolobenzodiazepine comprising two pyrrolobenzodiazepine units. In one embodiment, Z is a pyrrolobenzodiazepine comprising three pyrrolobenzodiazepine units. In one embodiment, Z is a pyrrolobenzodiazepine multimer comprising more than three pyrrolobenzodiazepine units. Structures of PBDs, as well as formulas and methods of producing them are described for example in PCT publications Nos: WO 2013/177481, WO 2011/130616, WO 2004/043880, WO 2005/085251, WO2012/112687 and WO 2011/023883, the disclosures of each of which are incorporated herein by reference.

The pyrrolo[2,1-c][1,4] benzodiazepines are a family of sequence-selective, minor-groove binding DNA-interactive agents that covalently attach to guanine residues. It has been reported that the (S)-chirality at the C11a-position of PBDs provides them with the appropriate 3-dimensional shape to fit perfectly into the DNA minor groove. PBDs can have different effects and modes of action. PBDs can be DNA-binders or DNA-alkylators that do not cause crosslinking of DNA, or PBDs can be DNA cross-linkers.

The pyrrolobenzodiazepine unit or monomer can have a general structure as follows:

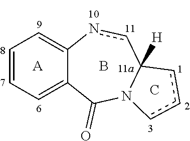

wherein the PBD can have different number, type and position of substituents, in both the aromatic A rings and pyrrolo C rings, and can vary in the degree of saturation of the C ring. In the B-ring there is either an imine (N═C), a carbinolamine (NH—CH(OH)), or a carbinolamine methyl ether (NH—CH(OMe)) at the N¹⁰—C¹¹ position which is the electrophilic centre responsible for alkylating DNA.

The biological activity of PBDs can be potentiated by joining two (or more) PBD monomers or units together, typically through their C8/C8′-hydroxyl functionalities via a flexible alkylene linker.

In one aspect of the any of the embodiments herein, a pyrrolobenzodiazepine monomer or unit is a pyrrolo[2,1-c][1,4]benzodiazepine. In one aspect of the any of the embodiments herein, a pyrrolobenzodiazepine dimer is a C8/C8′-linked pyrrolo[2,1-c][1,4]benzodiazepine dimer.

A PBD can be attached to a linker through any suitable position. For example, the PBD can be connected to a linker (e.g., to a Y or to V, or, when absent, to L in a compound of Formula II; or to a Y′ or to V′, or, when absent, to L′ in a compound of Formula III), via any of the positions in a PBD unit indicated below.

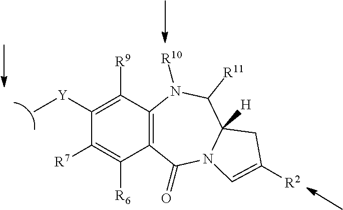

In one embodiment, a PBD dimer comprises the structure of the general formula below, with exemplary attachments points to other substituents or functionalities within a compound of Formula II or III indicated by arrows:

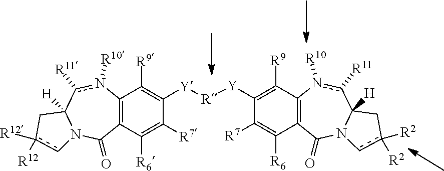

wherein:

R¹² and R^{12′}, and/or R² and R^{2′} together respectively form a double bond=CH₂ or ═CH—CH₃;
or
R^{2′} and R^{12′} are absent and R² and R¹² are independently selected from:
(iia) C₁₋₅ saturated aliphatic alkyl;
(iib) C₃₋₆ saturated cycloalkyl;
(iic)

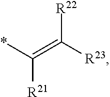

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;
(iid)

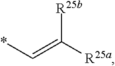

wherein one of R^{25a} and R^{25b} is H and the other is selected from:
phenyl, which phenyl is optionally substituted by a group selected from halo, methyl, methoxy; pyridyl; and thiophenyl; and
(iie)

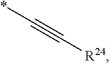

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;

R⁶ and R⁹ are independently selected from H, R, OH, OR, SH, SR, NH₂, NHR, NRR′, nitro, Me₃Sn and halo; where R and R′ are independently selected from optionally substituted C₁₋₁₂ alkyl, C₃₋₂₀ heterocyclyl and C₅₋₂₀ aryl groups;

R⁷ is selected from H, R, OH, OR, SH, SR, NH₂, NHR, NHRR′, nitro, Me₃Sn and halo;

either:

(a) R¹⁰ is H, and R¹¹ is OH, OR^{A}, where R^{A} is alkyl;

(b) R¹⁰ and R¹¹ form a nitrogen-carbon double bond between the nitrogen and carbon atoms to which they are bound; or

(c) R¹⁰ is H and R¹¹ is SO_{z}M, where z is 2 or 3 and M is a monovalent pharmaceutically acceptable cation;

R″ is a C₃₋₁₂ alkylene group, which chain may be interrupted by one or more heteroatoms, e.g., O, S, NR^{N2} (where R^{N2} is H or C₁₋₄ alkyl), and/or aromatic rings, e.g., benzene or pyridine;

Y and Y′ are selected from O, S, or NH; and

R^{6′}, R^{7′}, R^{9′} are selected from the same groups as R⁶, R⁷ and R⁹ respectively and

R^{10′} and R^{11′} are the same as R¹⁰ and R¹¹, wherein if R¹¹ and R¹¹ are SO_{z}M, M may represent a divalent pharmaceutically acceptable cation.

In another example, a PBD dimer comprises the structure of the general formula below:

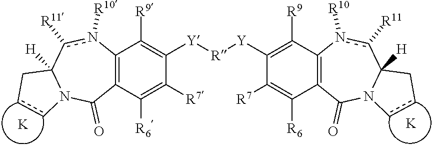

wherein R⁶, R⁷, R⁹, R^{6′}, R^{7′}, R^{9′}, R¹⁰, R¹¹, R^{10′} and R11′ are as defined above, and wherein the “K” ring is a substituted or unsubstituted aromatic or non-aromatic ring, optionally a 6-member ring, optionally a phenyl.

Examples of PBD dimers include:

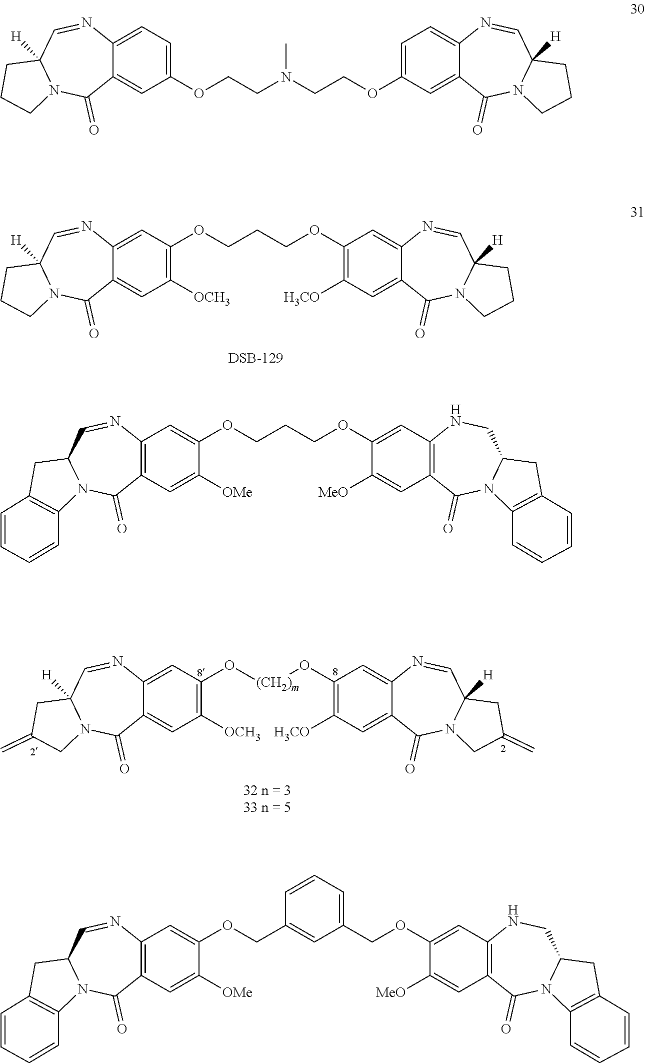

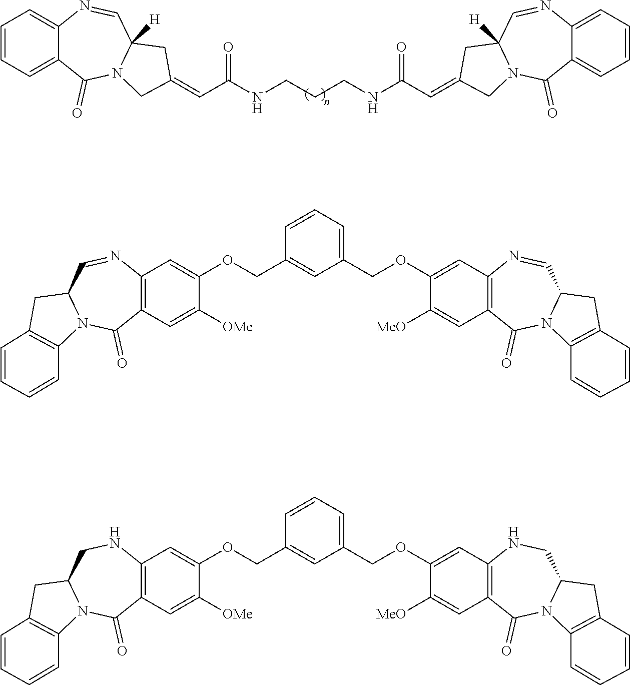

Constant Regions

In one embodiment, an immunoconjugate can be prepared such that it does not have substantial specific binding to human Fcγ receptors, e.g., any one or more of CD16A, CD16B, CD32A, CD32B and/or CD64). In one embodiment, they have no or low binding to each of human CD16A, CD16B, CD32A, CD32B and/or CD64. Antibodies may comprise constant regions of various heavy chains that are known to lack or have low binding to Fcγ receptors. Alternatively, antibody fragments that do not comprise (or comprise portions of) constant regions, such as F(ab′)2 fragments, can be used to avoid Fc receptor binding. Fc receptor binding can be assessed according to methods known in the art, including for example testing binding of an antibody to Fc receptor protein in an SPR (e.g. Biacore) assay. Also, generally any antibody IgG isotype can be used in which the Fc portion is modified (e.g., by introducing 1, 2, 3, 4, 5 or more amino acid substitutions) to minimize or eliminate binding to Fc receptors (see, e.g., WO 03/101485, the disclosure of which is herein incorporated by reference). Assays such as cell based assays, to assess Fc receptor binding are well known in the art, and are described in, e.g., WO 03/101485.

In one embodiment, an antigen binding protein (e.g. antibody) can comprise one or more specific mutations in the Fc region that result in “Fc silent” antibodies that have minimal interaction with effector cells. Silenced effector functions can be obtained by mutation in the Fc region of the antibodies and have been described in the art: N297A mutation, the LALA mutations, (Strohl, W., 2009, Curr. Opin. Biotechnol. vol. 20(6):685-691); and D265A (Baudino et al., 2008, J. Immunol. 181: 6664-69) see also Heusser et al., WO2012/065950, the disclosures of which are incorporated herein by reference. In one embodiment, an antibody comprises one, two, three or more amino acid substitutions in the hinge region. In one embodiment, the antibody is an IgG1 or IgG2 and comprises one, two or three substitutions at residues 233-236, optionally 233-238 (EU numbering). In one embodiment, the antibody is an IgG4 and comprises one, two or three substitutions at residues 327, 330 and/or 331 (EU numbering). Examples of silent Fc IgG1 antibodies are the LALA mutant comprising L234A and L235A mutation in the IgG1 Fc amino acid sequence. Another example of an Fc silent mutation is a mutation at residue D265, or at D265 and P329 for example as used in an IgG1 antibody as the DAPA (D265A, P329A) mutation (U.S. Pat. No. 6,737,056). Another silent IgG1 antibody comprises a mutation at residue N297 (e.g. N297A, N297S mutation), which results in aglycosylated/non-glycosylated antibodies. Other silent mutations include: substitutions at residues L234 and G237 (L234A/G237A); substitutions at residues S228, L235 and R409 (S228P/L235E/R409K,T,M,L); substitutions at residues H268, V309, A330 and A331 (H268Q/V309L/A330S/A331S); substitutions at residues C220, C226, C229 and P238 (C220S/C226S/C229S/P238S); substitutions at residues C226, C229, E233, L234 and L235 (C226S/C229S/E233P/L234V/L235A; substitutions at residues K322, L235 and L235 (K322A/L234A/L235A); substitutions at residues L234, L235 and P331 (L234F/L235E/P331S); substitutions at residues 234, 235 and 297; substitutions at residues E318, K320 and K322 (L235E/E318A/K320A/K322A); substitutions at residues (V234A, G237A, P238S); substitutions at residues 243 and 264; substitutions at residues 297 and 299; substitutions such that residues 233, 234, 235, 237, and 238 defined by the EU numbering system, comprise a sequence selected from PAAAP, PAAAS and SAAAS (see WO2011/066501).

In one embodiment, an antibody can comprise an Fc domain of human IgG1 origin, comprises a mutation at Kabat residue(s) 234, 235, 237, 330 and/or 331. One example of such an Fc domain comprises substitutions at Kabat residues L234, L235 and P331 (e.g., L234A/L235E/P331S or (L234F/L235E/P331S). Another example of such an Fc domain comprises substitutions at Kabat residues L234, L235, G237 and P331 (e.g., L234A/L235E/G237A/P331S). Another example of such an Fc domain comprises substitutions at Kabat residues L234, L235, G237, A330 and P331 (e.g., L234A/L235E/G237A/A330S/P331S). In one embodiment, the antibody comprises an Fc domain, optionally of human IgG1 isotype, comprising: a L234X₁ substitution, a L235X₂ substitution, and a P331X₃ substitution, wherein X₁ is any amino acid residue other than leucine, X₂ is any amino acid residue other than leucine, and X₃ is any amino acid residue other than proline; optionally wherein X₁ is an alanine or phenylalanine or a conservative substitution thereof; optionally wherein X₂ is glutamic acid or a conservative substitution thereof; optionally wherein X₃ is a serine or a conservative substitution thereof. In another embodiment, the antibody comprises an Fc domain, optionally of human IgG1 isotype, comprising: a L234X₁ substitution, a L235X₂ substitution, a G237X₄ substitution and a P331X₄ substitution, wherein X₁ is any amino acid residue other than leucine, X₂ is any amino acid residue other than leucine, X₃ is any amino acid residue other than glycine, and X₄ is any amino acid residue other than proline; optionally wherein X₁ is an alanine or phenylalanine or a conservative substitution thereof; optionally wherein X₂ is glutamic acid or a conservative substitution thereof; optionally, X₃ is alanine or a conservative substitution thereof; optionally X₄ is a serine or a conservative substitution thereof. In another embodiment, the antibody comprises an Fc domain, optionally of human IgG1 isotype, comprising: a L234X₁ substitution, a L235X₂ substitution, a G237X₄ substitution, G330X₄ substitution, and a P331X₅ substitution, wherein X₁ is any amino acid residue other than leucine, X₂ is any amino acid residue other than leucine, X₃ is any amino acid residue other than glycine, X₄ is any amino acid residue other than alanine, and X₅ is any amino acid residue other than proline; optionally wherein X₁ is an alanine or phenylalanine or a conservative substitution thereof; optionally wherein X₂ is glutamic acid or a conservative substitution thereof; optionally, X₃ is alanine or a conservative substitution thereof; optionally, X₄ is serine or a conservative substitution thereof; optionally X₅ is a serine or a conservative substitution thereof. In the shorthand notation used here, the format is: Wild type residue: Position in polypeptide: Mutant residue, wherein residue positions are indicated according to EU numbering according to Kabat.

In one embodiment, an antibody comprises an human IgG1 Fc domain comprising a L234A/L235E/N297X/P331S substitutions, L234F/L235E/N297X/P331S substitutions, L234A/L235E/G237A/N297X/P331S substitutions, or L234A/L235E/G237A/N297X/A330S/P331S substitutions, wherein X can be any amino acid other than an asparagine. In one embodiment, X is a glutamine; in another embodiment, X is not a residue other than a glutamine (e.g. a serine).

In one embodiment, an antibody comprises a heavy chain constant region comprising the amino acid sequence below, or an amino acid sequence at least 90%, 95% or 99% identical thereto but retaining the amino acid residues at Kabat positions 234, 235 and 331 (underlined):

(SEQ ID NO: 101)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E [symbol] G G P S V F L
F P P K P K D T L M I S R T P E V T C V V V D V S
H E D P E V K F N W Y V D G V E V H N A K T K P R
E E Q Y X S T Y R V V S V L T V L H Q D W L N G K
E Y K C K V S N K A L P A [symbol] I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K

In one embodiment, an antibody comprises a heavy chain constant region comprising the amino acid sequence below, or an amino acid sequence at least 90%, 95% or 99% identical thereto but retaining the amino acid residues at Kabat positions 234, 235 and 331 (underlined):

(SEQ ID NO: 102)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E [symbol] G G P S V F L
F P P K P K D T L M I S R T P E V T C V V V D V S
H E D P E V K F N W Y V D G V E V H N A K T K P R
E E Q Y X S T Y R V V S V L T V L H Q D W L N G K
E Y K C K V S N K A L P A [symbol] I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K

In one embodiment, an antibody comprises a heavy chain constant region comprising the amino acid sequence below, or an amino acid sequence at least 90%, 95% or 99% identical thereto but retaining the amino acid residues at Kabat positions 234, 235, 237, 330 and 331 (underlined):

(SEQ ID NO: 103)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E [symbol] G [symbol] S V F L F
P P K P K D T L M I S R T P E V T C V V V D V S H
E D P E V K F N W Y V D G V E V H N A K T K P R E
E Q Y X S T Y R V V S V L T V L H Q D W L N G K E
Y K C K V S N K A L P [symbol] I E K T I S K A K G Q P
R E P Q V Y T L P P S R E E M T K N Q V S L T C L
V K G F Y P S D I A V E W E S N G Q P E N N Y K T
T P P V L D S D G S F F L Y S K L T V D K S R W Q
Q G N V F S C S V M H E A L H N H Y T Q K S L S L
S P G K

In one embodiment, an antibody comprises a heavy chain constant region comprising the amino acid sequence below, or a sequence at least 90%, 95% or 99% identical thereto but retaining the amino acid residues at Kabat positions 234, 235, 237 and 331 (underlined):

(SEQ ID NO: 104)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E [symbol] G [symbol] P S V F L
F P P K P K D T L M I S R T P E V T C V V V D V S
H E D P E V K F N W Y V D G V E V H N A K T K P R
E E Q Y X S T Y R V V S V L T V L H Q D W L N G K
E Y K C K V S N K A L P A [symbol] I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K

In the sequence shown in SEQ ID NOS:101-104, X can be any amino acid, optionally a residue other than an asparagine. In one embodiment, X is a glutamine; in another embodiment, X is a residue other than a glutamine or an asparagine (e.g. X is an alanine or a serine).

Fc silent antibodies result in no or low ADCC activity, meaning that an Fc silent antibody exhibits an ADCC activity that is below 50%, optionally 30%, 20% or 10% specific cell lysis. Preferably an antibody substantially lacks ADCC activity, e.g., the Fc silent antibody exhibits an ADCC activity (specific cell lysis) that is below 5% or below 1%. Fc silent antibodies can also result in lack of FcγR-mediated cross-linking of MICA at the surface of a MICA-expressing cell.

In one embodiment, the antibody has a substitution in a heavy chain constant region at any one, two, three, four, five or more of residues selected from the group consisting of: 220, 226, 229, 233, 234, 235, 236, 237, 238, 243, 264, 268, 297, 298, 299, 309, 310, 318, 320, 322, 327, 330, 331 and 409 (numbering of residues in the heavy chain constant region is according to EU numbering according to Kabat). In one embodiment, the antibody comprises a substitution at residues 234, 235 and 322. In one embodiment, the antibody has a substitution at residues 234, 235 and 331. In one embodiment, the antibody has a substitution at residues 234, 235, 237 and 331. In one embodiment, the antibody has a substitution at residues 234, 235, 237, 330 and 331. In one embodiment, the Fc domain is of human IgG1 subtype. Amino acid residues are indicated according to EU numbering according to Kabat.

Uses in Therapy

Provided are compositions (e.g. pharmaceutical compositions) that comprise the immunoconjugates or antigen-binding proteins (e.g., antibodies) according to the invention. The immunoconjugates or antigen-binding proteins can be used to treat a variety of cancers characterized by MICA-expressing cells, e.g., malignant cells or MDSCs as may be found in tumor or tumor-adjacent tissues that express MICA (and optionally further MICB). For example the immunoconjugates can be used to treat an individual having a colorectal cancer, renal cell carcinoma, lung cancer (e.g. non-small cell lung carcinoma), melanoma, ovarian cancer, endometrial cancer, pancreatic cancer or a head and neck cancer. The antigen-binding protein, antibody or antibody fragment, via a cytotoxic agent bound thereto, can directly mediate the death, in vivo, of MICA positive cells such as cancer cells and/or MDSCs. The composition can be specified as comprising a pharmaceutically acceptable carrier.

The invention further provides a method of inhibiting the growth or activity of, and/or depleting, MICA-positive cells, in a patient in need thereof, comprising the step of administering to said patient a composition according to the disclosure. In one embodiment, provided is a method of delivering a cytotoxic agent to MICA-positive cells, in a patient in need thereof, comprising the step of administering to said patient a composition according to the disclosure. In one embodiment, provided is a method of increasing the biological activity (e.g. as assessed by increase proliferation of cells, pro-inflammatory cytokine production and/or markers of cytotoxicity, etc.) of effector cells (e.g. T cells, NK cells) that express NKG2D in an individual having a MICA-expressing cancer. Such treatment methods can be used for a MICA-expressing proliferative disorder, including, but not limited to the treatment of cancers.

In one aspect, the methods of treatment of the invention comprise administering to an individual a therapeutically effective amount of a composition comprising an antigen-binding compound of the disclosure that binds MICA. In one aspect, a therapeutically effective amount may be an amount sufficient to cause a detectable depletion (elimination) of MICA cells in vivo. In one aspect, the therapeutically effective amount is an amount that provides (e.g. achieves and/or maintains) a concentration in circulation (and/or in a tumor tissue) that corresponds to a concentration that causes a detectable depletion (death) of MICA-expressing cells (e.g. tumor cells) in vitro. In one aspect, the therapeutically effective amount is an amount that provides (e.g. achieves and/or maintains) a concentration in circulation (and/or in a tumor tissue) that corresponds to at least the EC₂₀, the EC₅₀, the EC₇₀ or the EC₁₀₀ for depletion of MICA cells (e.g. tumor cells) in vitro.

In one aspect, the therapeutically effective amount is furthermore an amount that does not cause a significant increase or induction in soluble MICA in circulation and/or a significant increase or induction in MICA shedding from tumor cells. In one aspect, the therapeutically effective amount is furthermore an amount that does not cause, or that prevents, a significant down-modulation of NKG2D receptor expression on the surface of immune effector cells (e.g. T cells, NK cells).

In one aspect, the therapeutically effective amount is an amount that provides (e.g. achieves and/or maintains) a concentration in circulation (and/or in a tumor tissue) that corresponds to a concentration that does not cause a significant increase or induction in soluble MICA and/or does not cause a significant increase or induction in MICA shedding from tumor cells, as assessed in an in vitro assay (e.g. in which MICA-expressing tumor cells are incubated with the anti-MICA antigen binding agent).

In one aspect, a therapeutically effective amount is an amount that does not significantly mediate ADCC towards MICA-expressing cells, for example the amount provides (e.g. achieves and/or maintains) a concentration in circulation (and/or in a tumor tissue) that is lower than the EC₅₀, the EC₂₀ or the EC₁₀ for ADCC, e.g. as assessed in an in vitro ADCC assay, or for example an amount that corresponds to a concentration that does not result in detectable ADCC as assessed in an in vitro cell cytotoxicity assay.

“EC₅₀” with respect to a particular biological activity (e.g. binding, mediating ADCC, depleting cells, for example) refers to the efficient concentration of an agent (e.g. anti-MICA immunoconjugate) which produces 50% of its maximum response or effect with respect to the particular biological activity. “EC₇₀” with respect to a biological activity, refers to the efficient concentration of an agent which produces 70% of its maximum response or effect with respect to such biological activity. “EC₁₀₀” with respect to the biological activity, refers to the efficient concentration of an agent which produces its substantially maximum response or effect with respect to such biological activity.

Determining the EC₂₀, the EC₅₀, the EC₇₀ or the EC₁₀₀ for depletion or cytotoxicity (e.g. direct, non-immune cell mediated cytotoxicity) towards MICA-expressing cells (e.g. tumor cells) in vitro can for example be carried out using any of the assays described herein (see, e.g., the method of Example 3).

Determining the EC₅₀, the EC₂₀ or the EC₁₀ for ADCC can for example be carried out using a as shown for example using a classical 4-h ⁵¹Cr-release assay. Briefly, the cytolytic activity of human NK cell line KHYG-1 (DSMZ (German Collection of Microorganisms and Cell Cultures, Braunschweig, Germany, product ref. ACC 725) transfected with human CD16 is assessed in a classical 4-h ⁵¹Cr-release assay in 96 well plates. C1R-MICA cells are labelled with ⁵¹Cr, then mixed with KHYG-transfected with hCD16 at an effector/target ratio equal to 20, in the presence of antibody at indicated concentrations and of 10 μg/ml F(ab′)² ON72 antibody (Beckman Coulter Inc.) to block any NKG2D-mediated cytotoxicity). After brief centrifugation and 4 hours of incubation at 37° C., 50 μL supernatant are removed, and the ⁵¹Cr release is measured with a TopCount NXT beta detector (PerkinElmer Life Sciences, Boston, Mass.). All experimental groups are analyzed in triplicate, and the percentage of specific lysis was determined as follows: 100×(mean cpm experimental release−mean cpm spontaneous release)/(mean cpm total release−mean cpm spontaneous release). Percentage of total release obtained by lysis of target cells with 2% Triton X100 (Sigma). See also, e.g., Example 11 of PCT publication no. WO2013/117649.

In one embodiment, the treatment does not cause an increase or induction of soluble (e.g. shed) MICA in circulation in an individual. In one embodiment, the treatment results in a decrease of soluble (e.g., shed) MICA in circulation in an individual. In one embodiment, the treatment does not result in a significant down-modulation of NKG2D receptor expression on the surface immune effector cells (e.g. T cells, NK cells). In one embodiment, the treatment results in an increase in NKG2D receptor expression on the surface of immune effector cells (e.g. T cells, NK cells).

In one embodiment, the treatment results in an increase in biological activity of effector cells that express NKG2D (e.g. NK and/or T cells), for example as assessed by increase proliferation of cells, pro-inflammatory cytokine production and/or markers of cytotoxicity, etc.

In one embodiment, an anti-MICA composition of the disclosure can advantageously be administered in an amount that achieves a concentration in circulation that is less than the concentration that achieves 50%, 20% or 10% receptor saturation (e.g., as assessed by titrating the anti-MICA composition on MICA-expressing cells, as assessed at e.g. 24 hours, 1 week, 2 weeks, 3 weeks or 1 month upon administration).

In one embodiment, an anti-MICA composition of the disclosure is administered in an amount that maintains a concentration (e.g. a trough concentration) that corresponds to at least the EC₂₀, the EC₅₀, the EC₇₀ or the EC₁₀₀ for depletion of MICA cells, and that provides less than 50%, 20% or 10% receptor saturation (e.g., as assessed by titrating the anti-MICA composition on MICA-expressing cells, at e.g. 24 hours, 1 week, 2 weeks, 3 weeks or 1 month upon administration). In one embodiment, the concentration is maintained until the subsequent administration of the composition. In one embodiment, the concentration is maintained for at least 1 week or at least 2 weeks.

The exemplary MICA binding protein used in the immunoconjugates of Examples 4 and 5 is antibody 19E9. The bivalent mean K_{D} (M) at pH 7.4 for MICA binding for biotin-conjugated mouse antibody 19E9 (on MICA*001-His) was 3.2*10¹³ M, while the monovalent affinity was 7.8*10⁻¹⁰ M (for detailed conditions, see Example 2 of WO2013/117647). Other antibodies can also be used similarly, for example antibodies characterized by a binding affinity (K_{D}), optionally wherein binding affinity is monovalent or bivalent, for a human MICA and/or MICB polypeptide (e.g. any one or more or all MICA and/or MICB alleles referred to herein, e.g. a MICA*001 polypeptide, a MICA*004 polypeptide, MICA*007 polypeptide and a MICA*008 polypeptide) of less than 10⁻⁹ M, preferably less than 10⁻¹⁰ M, preferably less than 10⁻¹¹ M, preferably less than 10⁻¹² M, or preferably less than 10⁻¹³M.

Antibody 19E9 is characterized by an EC₅₀, as determined by flow cytometry, of less than 2 μg/ml, for binding to C1R cells made to express at their surface a MICA protein (e.g., to each of a MICA*001 cell, a MICA*004 cell, a MICA*007 cell, and a MICA*008 cell, as further described herein). Other antibodies can also be used similarly, for example antibodies having an EC₅₀, as determined by flow cytometry, of less than 1 μg/ml, less than 0.5 μg/ml, or between 0.05 and 1 μg/ml, optionally between 0.001 and 1 μg/ml, or optionally between 0.01 and 0.2 μg/ml, for binding to cells made to express at their surface a MICA protein.

A composition of the disclosure can be administered, for example, at a dose of less than 1 mg/kg body weight, or at a dose of 0.05-0.5 mg/kg, optionally 0.01-0.1 mg/kg, 0.01-0.2 mg/kg, optionally 0.05-0.2 mg/kg, optionally 0.1 to 0.5 mg/kg, optionally 0.01 to 1 mg/kg, optionally 0.05 to 1 mg/kg or optionally 0.1 to 1 mg/kg body weight.

Exemplary treatment protocols for treating a human with a composition of the disclosure include, for example, administering to the patient an effective amount of with a composition of the disclosure, wherein the method comprises at least one administration cycle in which at least one dose of the composition is administered at a dose of less than 1 mg/kg body weight, or at a dose of 0.05-0.5 mg/kg, optionally 0.01-0.1 mg/kg, 0.01-0.2 mg/kg, optionally 0.05-0.2 mg/kg, optionally 0.1 to 0.5 mg/kg, optionally 0.01 to 1 mg/kg, optionally 0.05 to 1 mg/kg or optionally 0.1 to 1 mg/kg body weight. In one embodiment, the administration cycle is between 1 week and 8 weeks. In one embodiment, treatment with composition of the disclosure comprises at least one administration cycle in which the composition of the disclosure is administered at least twice in an effective amount as described herein, optionally at a dose of less than 1 mg/kg body weight, optionally at a dose of 0.05-0.5 mg/kg, optionally 0.01-0.1 mg/kg, 0.01-0.2 mg/kg, optionally 0.05-0.2 mg/kg, or optionally 0.1 to 0.5 mg/kg.

An exemplary treatment protocol for treating a human with a composition of the disclosure comprises administering the composition so at to achieve and/or maintain a continuous (minimum) blood concentration of the immunoconjugate corresponding to at least its EC₅₀ for cytotoxicity towards MICA-expressing tumor cells, optionally HCT116 cells. In another embodiment, an exemplary treatment protocol for treating a human with a composition of the disclosure comprises administering the composition so at to achieve and/or maintain a continuous (minimum) concentration in tumor tissue of the immunoconjugate corresponding to at least its EC₅₀ for cytotoxicity towards MICA-expressing tumor cells, optionally HCT116 cells. Optionally the concentration that is maintained in blood and/or tumor tissue is between 0.0005 and 0.01 μg/ml, optionally between 0.001 and 0.01 μg/ml. Optionally, the concentration is maintained between two subsequent administrations of the composition.

Optionally, at least 2, 3, 4, 5, 6, 7 or 8 doses of the composition of the disclosure are administered. In one embodiment, the composition of the disclosure is administered every week, every 2 weeks, every 3 weeks or every month.

The methods and compositions of the present invention can be utilized advantageously for the treatment of a variety of cancers, in particular MICA-expressing cancers, and other proliferative diseases including, but not limited to, carcinomas, colon cancer, bladder cancer, melanoma, lung cancer, hepatocellular cancer, glioblastoma, breast cancer, prostate cancer, hematological malignancies in general, acute myeloid leukemia, acute lymphatic leukemia, chronic myeloid leukemia and chronic lymphatic leukemia. More generally, exemplary cancers that can be treated include for example carcinomas, including that of the kidney, liver, head and neck, ovary, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma. Optionally, the cancer is a cancer characterized by cells (e.g. malignant cells, immunosuppressive cells) in the tumor environment or tumor tissue that express MICA and/or MICB proteins at their surface.

The treatment regimens and methods described herein may be used with or without a prior step of detecting the expression of MICA and/or MICB on cells in a biological sample obtained from an individual (e.g., a biological sample comprising cancer cells, cancer tissue or cancer-adjacent tissue). A patient whose tumor is characterized by cells that express MICA can then be treated with an anti-MICA antibody or composition. This can be accomplished by obtaining a sample of tumor cells from the site of the disorder, and testing e.g., using immunoassays, to determine the relative prominence of MICA and/or MICB and optionally further other markers on the cells. Other methods can also be used to detect expression of MICA and/or MICB and other genes, such as RNA-based methods, e.g., RT-PCR or Northern blotting. Optionally, soluble MICA and/or MICB is used as a marker for the presence of tumor cells expressing MICA and/or MICB at their surface. In one embodiment, a serum sample is obtained from an individual and the presence of soluble MICA and/or MICB is assessed, wherein a detection of soluble MICA and/or MICB in serum from an individual indicates that the individual has tumor comprising tumor cells that express MICA and/or MICB at their surface (membrane bound MICA and/or MICB).

In one embodiment, the disclosure provides a method for the treatment or prevention of a cancer in an individual in need thereof, the method comprising:

a) detecting cells (e.g. tumor cells, tumor infiltrating immune cells, tumor infiltrating macrophages) in a sample from the individual that express MICA and/or MICB, and

b) upon a determination that cells which express MICA and/or MICB are comprised in the sample, optionally at or above a reference level (e.g., a level corresponding to that of individuals having a benefit from treatment with an anti-MICA composition of the disclosure, administering to the individual an anti-MICA composition of the disclosure. The MICA and/or MICB reference level can be characterized by any suitable conventionally used reference level. For example, if at least 1%, optionally at least 5%, optionally at least 10%, optionally at least 50% of tumor cells or cells from a tumor tissue sample express MICA and/or MICB (e.g. using an immunohistochemistry-based assay), the sample can be determine to correspond to an individual that will derive substantial benefit from an agent of the disclosure. In one embodiment, the sample is from a tumor biopsy. In one embodiment, the same comprises tumor tissue and/or tumor-adjacent tissue.

In one embodiment, the disclosure provides a method for the treatment or prevention of a cancer in an individual in need thereof, wherein the individual has soluble MICA in circulation, optionally at elevated levels compared to a healthy individual. In one embodiment, the disclosure provides a method for the treatment or prevention of a cancer in an individual in need thereof, the method comprising:

a) determining whether the individual comprises circulating sMICA (e.g. obtaining a blood sample from the individual and detecting soluble MICA and/or MICB therein), and

b) upon a determination that the individual comprises circulating sMICA, optionally at or above a reference level (e.g., a level corresponding to a poor cancer prognosis, a level corresponding to that of individuals having a benefit from treatment with an anti-MICA composition of the disclosure, etc.), administering to the individual an anti-MICA composition of the disclosure.

The treatment may involve multiple rounds of administration. For example, following an initial round of administration, the level and/or activity of MICA-expressing cells (e.g., by detecting presence and/or levels of soluble MICA in serum of an individual), in an individual will generally be re-measured, and, if still elevated, an additional round of administration can be performed. In this way, multiple rounds of MICA detection and anti-MICA composition administration can be performed, e.g., until the disorder is brought under control.

In one embodiment, the treatment with an agent of the disclosure will result not cause a decrease in the frequency of activated, reactive, cytotoxic and/or IFNγ-production of NKG2D+ effector cells (e.g., NK cells) towards MICA-expressing tumor cells. In one embodiment, the treatment will result in an increase in the frequency of activated, reactive, cytotoxic and/or IFNγ-production of NKG2D+ effector cells (e.g., NK and/or T cells) towards MICA-expressing tumor cells.

The compositions described herein that are cytotoxic towards MICA and/or MICB-expressing cells without stabilizing sMICA or sMICB by protecting them from degradation can result in a progressive decrease of soluble MICA and/or MICB in circulation when the MICA and/or MICB-expressing tumor cells are eliminated. Consequently, the compositions and treatments can relieve sMICA and/or sMICB mediated host immunosuppression and thus be used advantageously in combination with immunotherapeutic agents to enhance an anti-cancer immune response, e.g. to enhance or stimulate an effector T cell and/or NK cell response against tumor cells. Such immunotherapeutic agents rely on the individual's immune system function for the ability to eliminate cancer cells and can be used beneficially with the compositions of the disclosure that can restore NKG2D expression and/or avoid down-regulation of NKG2D expression on immune effector cells.

Exemplary immunotherapeutic agents include antibodies that bind specifically to tumor antigens and are capable of mediating ADCC, as well as other depleting or non-depleting immunomodulatory agents such as agents that neutralize the inhibitory activity of inhibitory receptor on immune effector cells, or agents that activate the activating receptors on immune effector cells. In one example, the immunotherapeutic agent is an agent that neutralizes the inhibitory activity of PD-1. In one embodiment, the cancer is a head and neck squamous cell carcinoma, a non-small cell lung cancer (NSCLC), kidney cancer, gastrointestinal cancer, pancreatic or esophagus adenocarcinoma, renal cell carcinoma (RCC), melanoma, colorectal cancer or ovarian cancer, or a hematological malignancy.

Therapies for the treatment of a cancer using an agent of the disclosure that decreases MICA-mediated immunosuppression can advantageously involve administration of the MICA binding agent of the disclosure in combination with an immunotherapeutic agent, to treat subjects afflicted with cancer (e.g., advanced refractory or progressing solid or hematological tumors). In one embodiment, the invention provides a MICA binding agent of the disclosure and optionally further an immunotherapeutic agent in combination, for the treatment of subjects having a solid tumor characterized by MICA-expressing cells (e.g., a solid tumor, an advanced refractory solid tumor) or subjects having a hematological tumor characterized by MICA-expressing cells.

As used herein, combined administration includes simultaneous administration of the compounds in the same or different dosage form, as well as separate administration of the compounds (e.g., sequential administration). Thus, a MICA binding agent of the disclosure and an immunotherapeutic agent can be simultaneously administered in a single formulation. Alternatively, the MICA binding agent of the disclosure an immunotherapeutic agent can be formulated for separate administration and are administered concurrently or sequentially.

Accordingly, a MICA binding agent of the disclosure can be used to treat cancer in combination with a second or additional therapeutic agent.

In one embodiment, the second or additional therapeutic agent is an antibody or other Fc domain-containing protein capable of inducing ADCC toward a cell to which it is bound, e.g. via CD16 expressed by an NK cell. Typically, such antibody or other protein will comprise a domain that binds to an antigen of interest, e.g. an antigen present on a tumor cell (tumor antigen), and an Fc domain or portion thereof, and will exhibit binding to the antigen via the antigen binding domain and to Fcγ receptors (e.g. CD16) via the Fc domain. In one embodiment, its ADCC activity will be mediated at least in part by CD16. In one embodiment, the additional therapeutic agent is an antibody having a native or modified human Fc domain, for example a Fc domain from a human IgG1 or IgG3 antibody. The term “antibody-dependent cell-mediated cytotoxicity” or “ADCC” is a term well understood in the art, and refers to a cell-mediated reaction in which non-specific cytotoxic cells that express Fc receptors (FcRs) recognize bound antibody on a target cell and subsequently cause lysis of the target cell. Non-specific cytotoxic cells that mediate ADCC include natural killer (NK) cells, macrophages, monocytes, neutrophils, and eosinophils. The term “ADCC-inducing antibody” refers to an antibody that demonstrates ADCC as measured by assay(s) known to those of skill in the art. Such activity is typically characterized by the binding of the Fc region with various FcRs. Without being limited by any particular mechanism, those of skill in the art will recognize that the ability of an antibody to demonstrate ADCC can be, for example, by virtue of it subclass (such as IgG1 or IgG3), by mutations introduced into the Fc region, or by virtue of modifications to the carbohydrate patterns in the Fc region of the antibody. Examples of antibodies that induce ADCC include rituximab (for the treatment of lymphomas, CLL, trastuzumab (for the treatment of breast cancer), alemtuzumab (for the treatment of chronic lymphocytic leukemia) and cetuximab (for the treatment of colorectal cancer, head and neck squamous cell carcinoma). Examples of ADCC-enhanced antibodies include but are not limited to: GA-101 (hypofucosylated anti-CD20), margetuximab (Fc enhanced anti-HER2), mepolizumab, MEDI-551 (Fc engineered anti-CD19), obinutuzumab (glyco-engineered/hypofucosuylated anti-CD20), ocaratuzumab (Fc engineered anti-CD20), XmAb®5574/MOR208 (Fc engineered anti-CD19).

In one embodiment, the MICA binding agent of the disclosure augments the efficacy of an agent (e.g., an antibody) that inhibits CTLA-4 or the PD-1 axis (i.e. inhibits PD-1 or PD-L1). Antibodies that bind CTLA-4, PD1 or PD-L1 can be used, for example, at the exemplary the doses and/or frequencies that such agents are used as monotherapy, e.g., as described below.

In one embodiment, the second or additional second therapeutic agent is an agent (e.g., an antibody) that inhibits CTLA-4 or the PD-1 axis (i.e. inhibits PD-1 or PD-L1). Antibodies that bind CTLA-4, PD1 or PD-L1 can be used, for example, at the exemplary the doses and/or frequencies that such agents are used as monotherapy, e.g., as described below.

PD-1 is an inhibitory member of the CD28 family of receptors that also includes CD28, CTLA-4, ICOS and BTLA. PD-1 is expressed on activated B cells, T cells, and myeloid cells Okazaki et al. (2002) Curr. Opin. Immunol. 14: 391779-82; Bennett et al. (2003) J Immunol 170:711-8). Two ligands for PD-1 have been identified, PD-L1 and PD-L2, that have been shown to downregulate T cell activation upon binding to PD-1 (Freeman et al. (2000) J Exp Med 192:1027-34; Latchman et al. (2001) Nat Immunol 2:261-8; Carter et al. (2002) Eur J Immunol 32:634-43). PD-L1 is abundant in a variety of human cancers (Dong et al. (2002) Nat. Med. 8:787-9). The interaction between PD-1 and PD-L1 results in a decrease in tumor infiltrating lymphocytes, a decrease in T-cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well. Blockade of PD-1 can advantageously involve use of an antibody that prevents PD-L1-induced PD-1 signalling, e.g. by blocking the interaction with its natural ligand PD-L1. In one aspect the antibody binds PD-1 (an anti-PD-1 antibody); such antibody may block the interaction between PD-1 and PD-L1 and/or between PD-1 and PD-L2. In another aspect the antibody binds PD-L1 (an anti-PD-L1 antibody) and blocks the interaction between PD-1 and PD-L1.

There are currently at least six agents blocking the PD-1/PD-L1 pathway that are marketed or in clinical evaluation, any of these may be useful in combination with the anti-CD73 antibodies of the disclosure. One agent is BMS-936558 (Nivolumab/ONO-4538, Bristol-Myers Squibb; formerly MDX-1106). Nivolumab, (Trade name Opdivo®) is an FDA-approved fully human IgG4 anti-PD-L1 mAb that inhibits the binding of the PD-L1 ligand to both PD-1 and CD80 and is described as antibody 5C4 in WO 2006/121168, the disclosure of which is incorporated herein by reference. For melanoma patients, the most significant OR was observed at a dose of 3 mg/kg, while for other cancer types it was at 10 mg/kg. Nivolumab is generally dosed at 10 mg/kg every 3 weeks until cancer progression.

Another agent is durvalumab (Imfinzi®, MEDI-4736), an anti-PD-L1 developed by AstraZeneca/Medimmune and described in WO2011/066389 and US2013/034559.

Another agent is MK-3475 (human IgG4 anti-PD1 mAb from Merck), also referred to as lambrolizumab or pembrolizumab (Trade name Keytruda®) has been approved by the FDA for the treatment of melanoma and is being tested in other cancers. Pembrolizumab was tested at 2 mg/kg or 10 mg/kg every 2 or 3 weeks until disease progression.

Another agent is atezolizumab (Tecentriq®, MPDL3280A/RG7446, Roche/Genentech), a human anti-PD-L1 mAb that contains an engineered Fc domain designed to optimize efficacy and safety by minimizing FcγR binding and consequential antibody-dependent cellular cytotoxicity (ADCC). Doses of 51, 10, 15, and 25 mg/kg MPDL3280A were administered every 3 weeks for up to 1 year. In phase 3 trial, MPDL3280A is administered at 1200 mg by intravenous infusion every three weeks in NSCLC.

Further known PD-1 antibodies and other PD-1 inhibitors include Pidlizumab (CT-011; CureTech) (humanized IgG1 anti-PD1 mAb from CureTech/Teva), Pidlizumab (CT-011; CureTech) (see e.g., WO2009/101611), AMP-224 (a B7-DC/IgG1 fusion protein licensed to GSK), AMP-514 described in WO 2012/145493, antibody YW243.55.S70 (an anti-PD-L1) described in WO2010/077634, MDX-1105, also known as BMS-936559, is an anti-PD-L1 antibody developed by Bristol-Myers Squibb described in WO2007/005874, and antibodies and inhibitors described in WO2006/121168, WO2009/014708, WO2009/114335 and WO2013/019906, the disclosures of which are hereby incorporated by reference. Further examples of anti-PD1 antibodies are disclosed in WO2015/085847 (Shanghai Hengrui Pharmaceutical Co. Ltd.), for example antibodies having light chain variable domain CDR1, 2 and 3 of SEQ ID NO: 6, SEQ ID NO: 7 and/or SEQ ID NO: 8, respectively, and antibody heavy chain variable domain CDR1, 2 and 3 of SEQ ID NO: 3, SEQ ID NO: 4 or SEQ ID NO: 5, respectively, wherein the SEQ ID NO references are the numbering according to WO2015/085847, the disclosure of which is incorporated herein by reference. Antibodies that compete with any of these antibodies for binding to PD-1 or PD-L1 also can be used.

CTLA-4 (cytotoxic T-lymphocyte-associated protein 4), also known as CD152 is another inhibitory member of the CD28 family of receptors, and is expressed on T cells. Antibodies that bind and inhibit CTLA-4 are known in the art. In one example, the antibody is ipilimumab (trade name Yervoy®, Bristol-Myers Squibb), a human IgG antibody. In one example, the antibody is tremelimumab (CP-675,206; Medimmune/AstraZeneca). An exemplary administration regimen for Yervoy is 3 mg/kg invtravenously over 90 minutes every three weeks. In one example, the antibody used in combination with the anti-CD73 antibodies of the disclosure is an antibody that competes with ipilimumab or tremelimumab for binding to CTLA-4.

The MICA-binding immunoconjugate of the disclosure that reduce immunosuppression can also be used advantageously to treat cancer in an individual who is a poor responder to treatment with an immunomodulatory agent, such as an agent that binds a tumor antigen and mediates ADCC, or an agent that neutralizes the inhibitory activity of PD-1, for example, an individual who experiences or is predicted to have a high likelihood to experience (e.g. based on one or more prognostic factors) an incomplete response, lack of therapeutic response, detectable or residual cancer and/or progressive disease upon (during or following) treatment with the agent (e.g. an agent that neutralizes the inhibitory activity of PD-1). In one embodiment, the individual's cancer has not experienced a complete response or is predicted to have a likelihood (e.g. a high likelihood) not to experience a complete response upon (during or following) treatment with an agent that neutralizes the inhibitory activity of PD-1. In one embodiment, the individual's cancer has progressed (e.g. progressive disease) upon (during or following) treatment with an agent that neutralizes the inhibitory activity of PD-1. In one embodiment, the individual's cancer has partially responded or stabilized (partial response or stable disease) but is predicted to have a likelihood to progress upon (during or following) treatment with an agent that neutralizes the inhibitory activity of PD-1.

In one example, the individual has a cancer known to be poorly responsive to treatment with an agent that neutralizes the inhibitory activity of PD-1 (e.g. in monotherapy). Optionally the cancer is a head and neck squamous cell carcinoma, a non-small cell lung cancer (NSCLC), kidney cancer, gastrointestinal cancer, pancreatic or esophagus adenocarcinoma, breast cancer, renal cell carcinoma (RCC), melanoma, colorectal cancer or ovarian cancer. Such an individual can optionally furthermore be advantageously be treated with a MICA-binding immunoconjugate of the disclosure in combination with an agent that neutralizes the inhibitory activity of PD-1.

For example, the individual may have a cancer that is poorly responsive (or resistant or non-responsive), for example a cancer that has relapsed or progressed despite (e.g. during or following) treatment with an agent that neutralizes the inhibitory activity of PD-1. In one embodiment, the individual treated with a MICA-binding immunoconjugate has experienced an incomplete response (has not experienced a complete response (CR)) upon (during or following) treatment with an agent that neutralizes the inhibitory activity of PD-1 (e.g. as monotherapy or as combination therapy with an agent other than a MICA-binding immunoconjugate), or has experienced at least a partial response (PR) upon (during or following) treatment with an agent that neutralizes the inhibitory activity of PD-1, but whose cancer has relapsed or progressed. In any embodiment herein, treatment response can be defined and/or assessed according to well-known criteria, e.g. Response Evaluation Criteria In Solid Tumors (RECIST), such as version 1.1, see Eisenhauer et al. (2009) Eur. J. Cancer 45:228-247, or Immune-Related Response Criteria (irRC), see Wolchock et al. (2009) Clinical Cancer Research 15:7412-7420.

In one embodiment, an individual who is a poor responder (or who has a cancer that is poorly responsive) is an individual having a poor disease prognosis for treatment with an agent that neutralizes the inhibitory activity of PD-1. An individual having a poor disease prognosis can, for example, be determined to have a high or higher risk of cancer progression (e.g. compared to individuals having a good disease prognostic), based on one or more predictive factors. In one embodiment, a predictive factor(s) comprises presence or absence of a mutation in one or more genes. In one embodiment, the mutation defines a neo-epitope recognized by a T cell. In one embodiment, the predictive factor(s) comprises level(s) of expression of one or more genes or proteins in tumor cells, e.g. PD-L1, decreased or elevated levels of PD-L1 on tumor cells. In one embodiment, the predictive factor(s) comprises level(s) of expression of one or more genes or proteins in NK and/or CD8 T cells in circulation or in the tumor environment, e.g., PD-1. In one embodiment, the predictive factor(s) comprises mutational load in cancer cells, e.g. number of non-synonymous mutations per exome.

In the treatment methods, the MICA-binding compound and the second therapeutic agent can be administered separately, together or sequentially, or in a cocktail. In some embodiments, the antigen-binding compound is administered prior to the administration of the second therapeutic agent. For example, the MICA-binding compound can be administered approximately 0 to 30 days prior to the administration of the second therapeutic agent. In some embodiments, an MICA-binding compound is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days prior to the administration of the second therapeutic agent. In some embodiments, a MICA-binding compound is administered concurrently with the administration of the therapeutic agents. In some embodiments, a MICA-binding compound is administered after the administration of the second therapeutic agent. For example, a MICA-binding compound can be administered approximately 0 to 30 days after the administration of the second therapeutic agent. In some embodiments, a MICA-binding compound is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days after the administration of the second therapeutic agent.

Therapeutic formulations of compounds are prepared for storage by mixing the compound (e.g., antibody) having the desired degree of purity with optional pharmaceutically acceptable carriers, excipients, or stabilizers in the form of lyophilized formulations or aqueous solutions. For general information concerning formulations, see, e.g., Gilman et al. (eds.), The Pharmacological Bases of Therapeutics, 8^{1h} Ed. (Pergamon Press, 1990); Gennaro (ed.), Remington's Pharmaceutical Sciences, 18^{th} Edition (Mack Publishing Co., Easton, Pa., 1990); Avis et al. (eds.), Pharmaceutical Dosage Forms: Parenteral Medications (Dekker, New York, 1993); Lieberman et al. (eds.), Pharmaceutical Dosage Forms: Tablets (Dekker, New York, 1990); Lieberman et al. (eds.) Pharmaceutical Dosage Forms: Disperse Systems (Dekker, New York, 1990); and Walters (ed.), Dermatological and Transdermal Formulations (Drugs and the Pharmaceutical Sciences), Vol 119 (Dekker, New York, 2002).

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as ethylenediaminetetraacetic acid (EDTA); sugars such as sucrose, mannitol, trehalose, or sorbitol; salt-forming counter-ions such as sodium; metal complexes (e.g., Zn-protein complexes); and/or non-ionic surfactants such as TWEEN™, PLURONICS™, or PEG.

Lyophilized formulations adapted for subcutaneous administration are described, for example, in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

The formulation herein may also contain more than one active compound (a second medicament as noted above), preferably those with complementary activities that do not adversely affect each other. The type and effective amounts of such medicaments depend, for example, on the amount and type of compound present in the formulation, and clinical parameters of the subjects. The preferred such second medicaments are noted above.

Further aspects and advantages of this invention will be disclosed in the following experimental section, which should be regarded as illustrative and not limiting the scope of this application.

### EXAMPLES

### Example 1

### Production of ADCs

Antibodies 6E4, 9C10, 2006, 19E9, 18E8 having the murine VH and VL listed in Table 1 each bind with high affinity to MICA and MICB proteins, including MICA*001, *004, *007 and *008 alleles (as expressed by transfected human C1R cells). For this example, antibodies 6E4, 9C10, 2006, 19E9, 18E8 were produced as chimeric antibodies bearing human IgG1 constant regions, and deglycosylated with PNGaseF (New England Biolabs, 800U/mg of mAb) overnight at 37° C. Completion of the reaction was controlled by LC-ESI-MS.

A two-step process based on use of Click chemistry reactive groups was used in which a lysine based linker with a reactive group is first bound to the acceptor glutamines of the antibodies (in the absence of organic solvent), followed by reaction with a second compound that includes the PBD and a complementary reactive group (in the presence of organic solvent). To obtain the intermediate antibody bound to a reactive linker, 1 mg/mL deglycosylated mAb was incubated with 20 equivalents of a reactive lysine based linker (amino-PEG-azide, structure below) per site of coupling and 5 U/mL BTG overnight at 37° C. in PBS. The antibodies all comprised an acceptor glutamine at amino acid residue 295 (Kabat EU numbering) of their heavy chains, such that the reactive linker was conjugated to residue Q295 on each heavy chain of the antibodies (each antibody has two conjugated moieties; DAR=2), and lacking significant effector function.

The structure of the reactive linker was as follows:

NH2-PEG-N3 spacer:

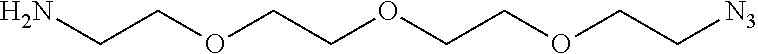 [SMILES: [N-]=[N+]=NCCOCCOCCOCCN]

The mAb-reactive linker conjugate was purified by affinity chromatography on protA.

Production of DNA Minor Groove Binder ADCs

In order to produce the final ADC comprising a pyrrolobenzodiazepine (PBD) dimer, the azide-functionalized antibodies above (2 mg/mL in PBS/1,2-propane-diol 50/50 v/v) were then incubated with 1.75 molar equivalent of DBCO-derivatized-PBD per site of coupling (the DBCO reacts with the azide). The mixture was incubated for 48-72h at RT with gentle agitation. Completion of the reaction was controlled by LC-ESI-MS. Excess of derivatized-PBD was removed by dialysis (MWCO=10 kDa), followed by purification by size exclusion chromatography (Superdex 200 10/300GL column, GEHealthcare). The final compounds were concentrated on Amicon 30K devices.

The structure of the DBCO-derivatized-PBD compound is shown as follows:

### DBCO-val-ala-PBD:

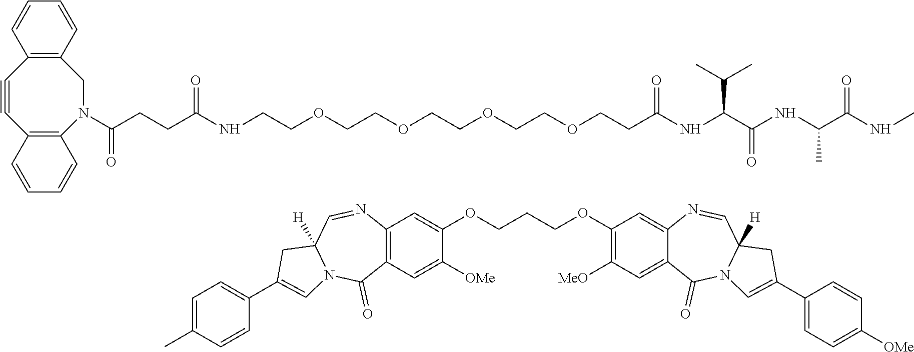

The final structure of the compound linked to the glutamine (represented by “mAb”) is shown as follows:

mAb-PEG-DBCO-PBD:

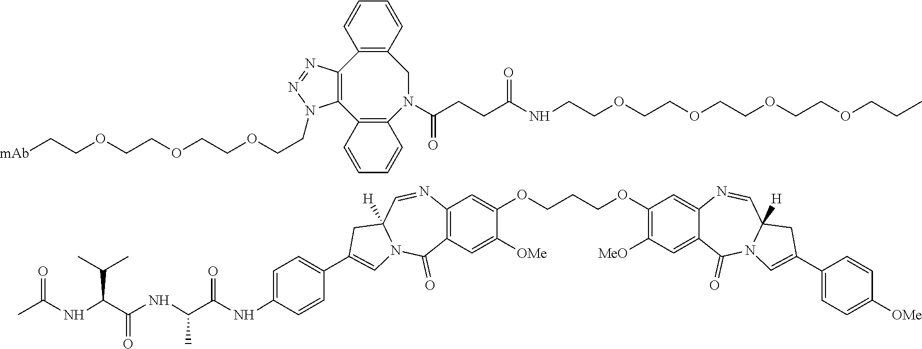

Production of Auristatin ADCs

In order to produce the final ADC comprising a monomethyl auristatin E, the azide-functionalized antibodies above (2 mg/mL in PBS) were then incubated with 1.5 molar equivalent of DBCO-derivatized-MMAE per site of coupling (the DBCO reacts with the azide). The mixture was incubated overnight at RT with gentle agitation. Completion of the reaction was controlled by LC-ESI-MS. Excess of derivatized-MMAE was removed by purification by size exclusion chromatography (Superdex 200 10/300GL column, GEHealthcare). The final compounds were concentrated on Amicon 30K devices.

The structure of the DBCO-derivatized-MMAE compound is shown as follows:

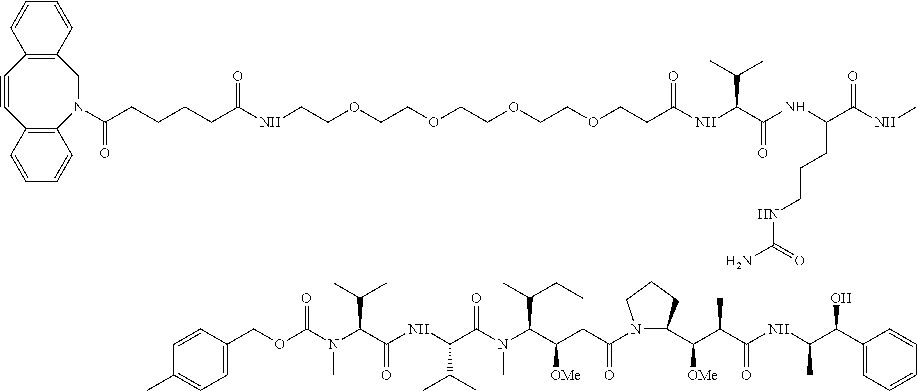

The final structure of the compound linked to the glutamine (represented by “mAb”) is shown as follows:

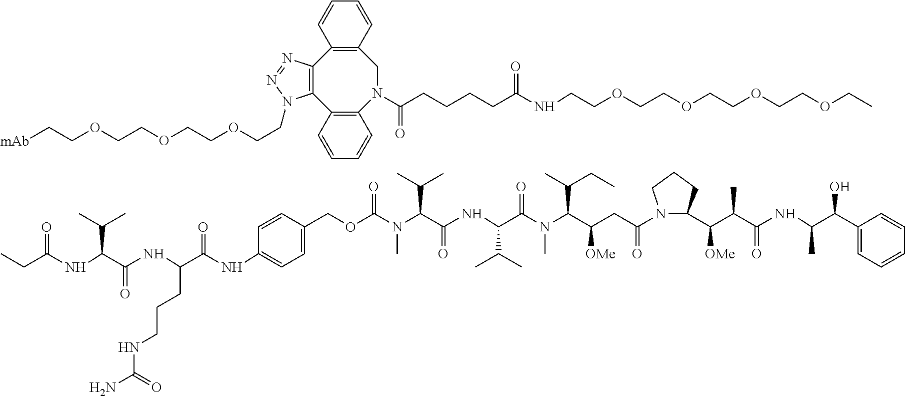

### Example 2

### Production of ADCs Having N297S Mutations

For this example, antibody 19E9, was produced as chimeric antibodies bearing human IgG1 constant regions comprising an asparagine to serine substation at Kabat heavy chain residue 297 (N297S), thereby eliminating native N297-linked glycosylation.

A two-step process based on use of Click chemistry reactive groups was used in which a lysine based linker with a reactive group is first bound to the acceptor glutamines of the antibodies (in the absence of organic solvent), followed by reaction with a second compound that includes the PBD and a complementary reactive group (in the presence of organic solvent). To obtain the intermediate antibody bound to a reactive linker, 5 mg/mL mutant mAb was incubated with 20 equivalents of a reactive lysine based linker (amino-PEG-azide, structure below) per site of coupling and 2 U/mL BTG overnight at 37° C. in PBS. The antibody comprised an acceptor glutamine at amino acid residue 295 (Kabat EU numbering) of their heavy chains, such that the reactive linker was conjugated to residue Q295 on each heavy chain of the antibodies (each antibody has two conjugated moieties; DAR=2), and lacking significant effector function.

The structure of the reactive linker was as follows:

NH2-PEG-N3 spacer:

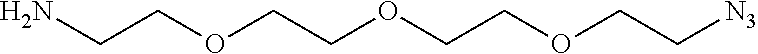 [SMILES: [N-]=[N+]=NCCOCCOCCOCCN]

The mAb-reactive linker conjugate was purified by affinity chromatography on protA.

Production of DNA Minor Groove Binder ADCs

In order to produce the final ADC comprising a pyrrolobenzodiazepine (PBD) dimer, the azide-functionalized antibody above (2 mg/mL in PBS/1,2-propane-diol 50/50 v/v) was then incubated with 1.75 molar equivalent of DBCO-derivatized-PBD per site of coupling (the DBCO reacts with the azide). The mixture was incubated for 48-72h at RT with gentle agitation. Completion of the reaction was controlled by LC-ESI-MS (DAR >1.9). Excess of derivatized-PBD was removed by dialysis (MWCO=10 kDa), followed by purification by size exclusion chromatography (Superdex 200 10/300GL column, GEHealthcare). The final compounds was concentrated on Amicon 30K devices.

The structure of the DBCO-derivatized-PBD compound is shown as follows:

### DBCO-val-ala-PBD:

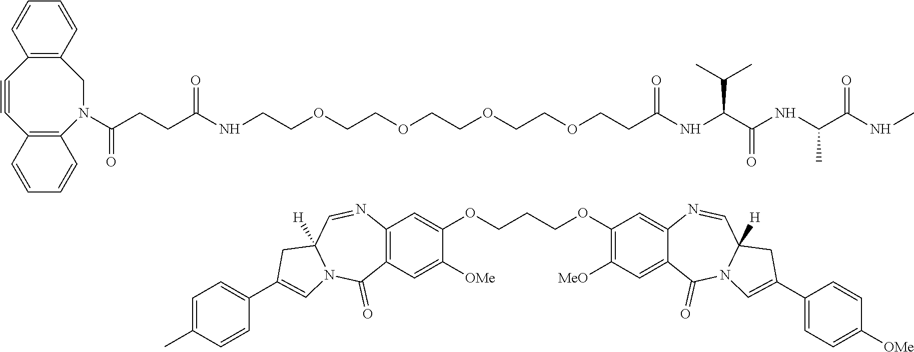

The final structure of the compound linked to the glutamine (represented by “mAb”) is shown as follows:

mAb-PEG-DBCO-PBD:

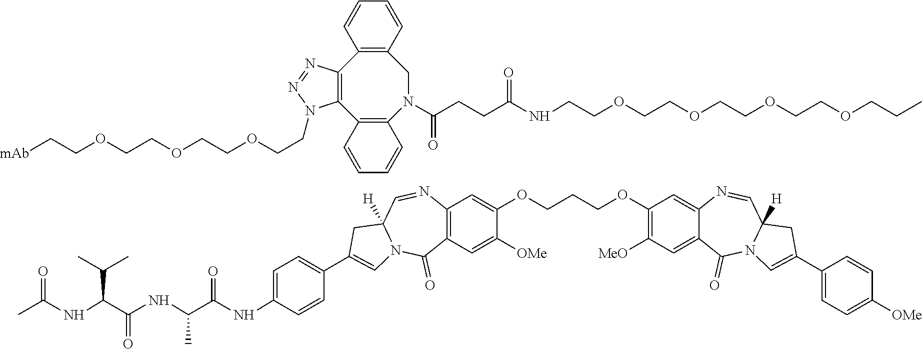

Determination of DAR by LC-ESI-MS

Drug Antibody Ratio (DAR) of ADCs was determined by LC-ESI-MS analysis of the reduced antibody. ADCs were reduced with DTT. ADC products were eluted on a BEH300-C4 (2.1×50 mm, 1.7 μm, Waters) heated to 80° C., at a flowrate of 0.2 mL/min, using the following gradient: 0-2 min, 10% B; 2-20 min, 10-40% B; 20-21 min, 40-90% B; 21-24 min, 90%; 24-25 min, 90-10% B; 25-30 min, 10% B (A: water+0.1% formic acid; B: acetonitrile+0.1% formic acid). Analytes were ionized by electrospray and detected by a Xevo G2S_QTof mass spectrometer (Waters) operating in positive TOF-MS mode. Raw data were analyzed with MassLynx software (Waters) and deconvolution was performed using MaxEnt1.

### Example 3

### Effector Function-Independent Anti-Tumoral Efficacy of DNA Minor Groove Binders in HCT116 Colon Carcinoma Model

### Cell Lines and Culture Media:

HCT116 (colon carcinoma) cells grown in RPMI 1640 Medium (Life Technologies SAS) supplemented with 10% decomplemented FBS (Life Technologies SAS)+2 mM L-Glutamine (Life Technologies SAS)+1 mM Sodium pyruvate (Life Technologies), at 37° C., 5% CO2.

### Antibody Drug Conjugates (ADCs):

Anti human MICA/B or negative control antibodies (Ab) were coupled with the PBD or MMAE toxins at a Drug/Ab ratio ˜2 as described in Example 2. The Ab clones used are: 6E4, 9C10, 2006, 19E9, 18E8 and Ab isotype control.

### Cytotoxicity Experiments:

Efficacy of PBD- or MMAE-coupled anti-MICA/B (or isotype control) antibodies (ADCs) was assessed by measuring cell confluence in the wells of 96-well plates over time using the IncuCyte™ Zoom™ apparatus (microscope within an incubator allowing pictures of each wells every 3 hours)Cytometry)(Essen Biosciences, Ann Arbor, Mich.). 2500 HCT116 cells were seeded in 100 μL of culture medium per well in flat bottom 96-wells plate (Corning) and cells were incubated for 4 hours at 37° C., 5% CO₂. 100 μL of 2× concentrated ADCs are added per wells (triplicates) in order to obtain 4 or 8 different final concentrations ranging from 10 μg/ml to 1 ng/ml+0 μg/ml control (depending on the toxin). Cells were then incubated at 37° C., 5% CO₂, for one week and four pictures/wells were taken every 6 hours during this week. Percentage of cell confluence at different time points were obtained using IncuCyte Zoom software and Area Under the Curve vs Log [ADCs] as well as EC₅₀ (also referred to as IC₅₀) were obtained using GraphPad Prism software.

### Cytometry Experiments:

MICA/B expression on cells was evaluated at the beginning of the cytotoxicity experiment using 100,000 cells/point. Cells were incubated on ice in 100 μl of FACS buffer (PBS 1×, SVF 0.2%, EDTA 2 mM, 0.22 μm filtered) containing either 10 μL of APC-conjugated anti-MICB or mouse IgG2b isotype control clones 236511 and 133303 respectively, R&D systems) or 1/10 of PE-conjugated anti MICA/B or isotype control (clones 19E9 and 13E4 respectively). Cells were washed twice and resuspended in FACS buffer containing 1/10000 SYTOX Blue Dead Cell Stain (Molecular Probes) just before using the cytometer (BD FACSCanto 10-Color System—BD Biosciences). Data were analysed using FlowJo software.

### Results:

Antibodies 6E4, 9C10, 2006, 19E9, 18E8 conjugated to the cytotoxic agent MMAE or a DNA minor groove binding agent (a PBD dimer) produced in Example 1 were tested for their ability to deplete HCT116 colon cancer cells. Antibodies were compared at a homogeneous drug:antibody ratio of about 2, wherein each antibody had one MMAE moiety bound to an amino acid residue in the CH2 domain of each heavy chain.

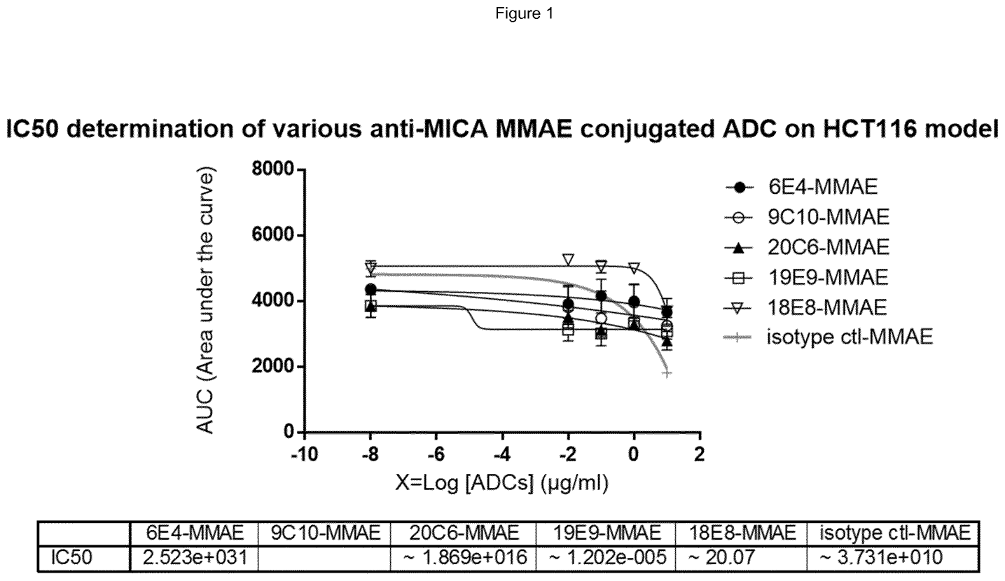

Results using MMAE-immunoconjugates are shown in FIG. 1. FIG. 1 shows the cytotoxicity of immunoconjuates towards HCT116 cells as a function of the concentration of the immunoconjugate, as well as the 10₅₀ (μg/ml) determination of MMAE-immunoconjugates. MMAE-immunoconjugates were tested at 0 μg/ml, 0.1 μg/ml, 0.5 μg/ml, 1 μg/ml and 10 μg/ml, however the MMAE-immunoconjugates did not show an ability at any concentration to deplete HCT116 cells compared to isotype control antibody conjugated to MMAE.

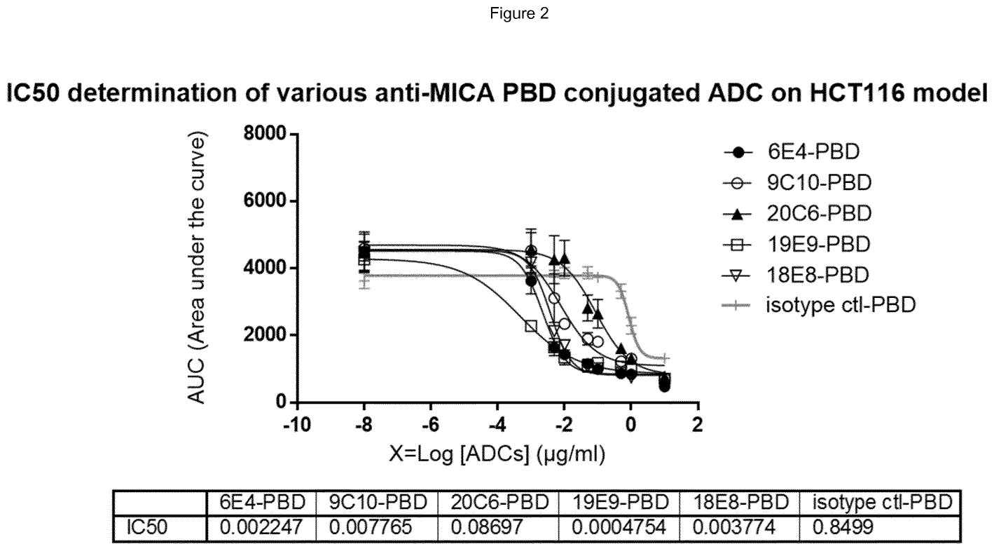

Results using PBD-immunoconjugates are shown in FIG. 2. FIG. 2 shows the cytotoxicity of immunoconjuates towards HCT116 cells as a function of the concentration of the immunoconjugate, as well as the IC₅₀ (μg/ml) determination of PBD-immunoconjugates. PBD-immunoconjugates were tested at 0 μg/ml, 0.0005 μg/ml, 0.001 μg/ml, 0.005 μg/ml, 0.01 μg/ml, 0.05 μg/ml, 0.1 μg/ml, 0.5 μg/ml, 1 μg/ml and 10 μg/ml.

Fluorescence intensity of MICA/B staining on HCT116 cells was verified by cytometry for QC.

In contrast to the MMAE-immunoconjugates which did not show significant efficacy, the DNA minor groove binder (PBD) immunoconjugates showed a strong increase in cytotoxicity compared to isotype control-PBD; the antibodies had particularly low EC₅₀ values, moreover surprising in view of the low DAR of 2. Furthermore, the antibody 19E9-based DNA minor groove binder immunoconjugate had a particularly low EC₅₀ of 0.0004754 μg/ml, followed by antibody 18E8 (EC₅₀ of 0.003774 μg/ml), antibody 6E4 (EC₅₀ of 0.002247 μg/ml) and antibody 9C10 (EC₅₀ of 0.007765 μg/ml).

### Example 4

### Efficacy of ADCs in NMRI Nude Mice Engrafted with HCT116 2M+Matrigel SC

Anti-MICA/B immunoconjugate produced with the N297S substitution based on antibody 19E9 was conjugated to DNA minor groove binding agent (PBD) as described in Example 1 was tested in a mouse NMRI tumor model.

HCT116 human colorectal carcinoma cells (ATCC ref CCL-247) which endogenously express MICA on their surface were harvested using PBS1X+2 mM EDTA and cultured in complete medium containing RPMI supplemented with 10% of Fetal Bovine Serum Heat Inactivated, 1% of L-glutamine and 1% Sodium Pyruvate. The cells were used after 5 or 6 passages of subculture; At each step of subculturing, cells were counted using a Malassez hemacytometer, and their viability was recorded using Trypan Blue coloration. The day of engraftment cells were centrifuged, washed in PBS 1X (Gibco; Life science Invitrogen) and resuspended to obtain the concentration needed. Cells were engrafted SC in 200 μL with Matrigel with a 27.5G needle (Matrigel Basement Membrane Matrix; BD Biosciences). Cells had a minimum of 85% viability to be engrafted. When tumors appeared, they were measured and groups with homogeneous and comparable tumor volume were formed for efficacy studies.

When tumors reached 50-100 mm³, mice were randomized for treatment intraperitoneally (i.p.) with either isotype control antibody (IC) or chimeric 19E9 antibody-PBD immunoconjugate as described in Example 2. Immunoconjugates were administered in a final volume of 100 μl. Immunoconjugates were administered at the dosage 0.05 mg/kg, 0.1 mg/kg or 0.2 mg/kg body weight. Mice receiving 0.05 mg/kg were treated once per week for 3 weeks. Mice receiving either 0.1 mg/kg or 0.2 mg/kg were treated once (once per week for one week). Each group included 10 mice. The tumor volumes were monitored by measuring length (A) and width (B) were measured using a digital caliper twice/week. The tumor volume was calculated as followed: (A×(B)2/2).

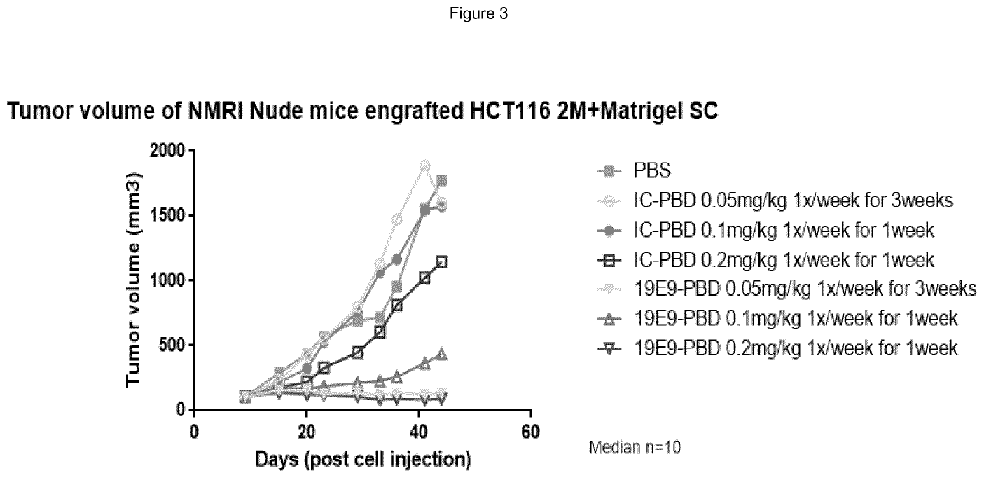

Results are shown in FIG. 3 (mean of 10 mice for each group). The anti-MICA-PBD immunoconjugates showed strong anti-tumoral activity even at the low doses, including at each of 0.05 mg/kg, 0.1 mg/kg or 0.2 mg/kg body weight, and effectively prevented increase in tumor volume in all mice and led to elimination of tumors. Of note, in the setting of repeat administration (the 0.05 mg/kg dose), even the lowest dose led to substantially full elimination of tumors.

### Example 5: Antibody-Mediated Stabilization of Soluble MICA In Vivo

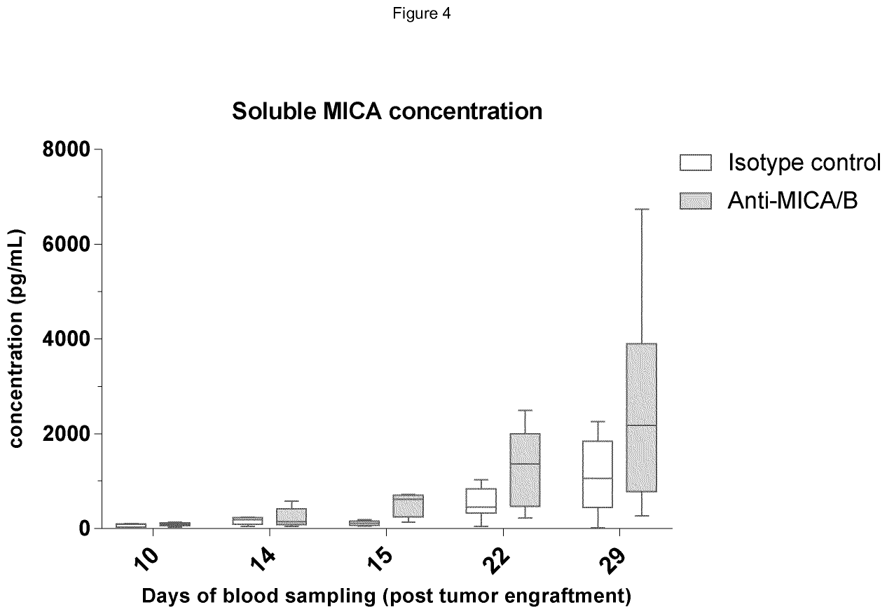

In order to define whether the anti-MICA antibody can stabilize soluble MICA, the following experiment was designed. CB17-SCID mice were engrafted s.c. with 15 million Raji-soluble MICA*001 tumor cell line. This cell line was engineered to secrete the extracellular domain of MICA*001 and does not express MICA*001 at the cell surface. Therefore, the anti-MICA antibody can only bind to circulating soluble MICA*001 and cannot bind to the tumor cells. 10 days after engraftment, mice were randomized in two treatment groups (10 mice per group). One group was treated with an isotype control, the other with the anti-MICA/B antibody produced as a human IgG1 isotype. 250 μg of antibodies were administered i.p. once a week. Tumor growth was similar in both treatment groups indicating that the anti-MICA antibody has no cytotoxic effect against a tumor cell not expressing MICA at the cell surface, as expected. Blood sampling was performed at indicated days to measure circulating soluble MICA concentration by ELISA. The ELISA was developed to detect MICA even in the presence of the anti-MICA antibody. FIG. 4 shows that upon treatment with anti-MICA antibody, more soluble MICA is detected in the anti-MICA antibody-treated group, indicating that soluble MICA half-life in the blood circulation is increased upon binding to anti-MICA antibodies.

### Example 6

### Efficacy of ADCs in Human Breast Cancer PDX Mouse Model Compared to Standard of Care

A murine patient-derived xenograft (PDX) model of cancer was used to evaluate anti-MICA/B immunoconjugate based on antibody 19E9 antibody conjugated to a DNA minor groove binding agent (PBD) as described in Example 1. The model makes use of SHO™ mice (Charles River Laboratories, Inc.), a result of intercrossing Crl:HA-Prkdcscid and Crl:SKH1-Hrhr stocks. The resulting animals are homozyogous for the Prkdcscid and the Hrhr mutations and thus exhibit the severe combined immunodeficiency phenotype characteristic of SCID mice and are also hairless.

SHO™ mice (groups of 10) were engrafted with HBCx-5 human breast cancer cells and randomized for treatment intraperitoneally (i.p.) with either bevacizumab (trade name: Avastin™), or isotype control antibody (IC) or chimeric 19E9 antibody bearing the N297S mutation on a human IgG1 Fc domain conjugated to DNA minor groove binder (PBD) at DAR 2 as described in Example 2. 48 mice with HBCx-5 subcutaneous growing tumor (P21.1.2/0) between 40 and 196 mm3 were allocated, according to their tumor volume to give homogenous mean and median tumor volume in each treatment arm. Bevacizumab was administered twice per week at 5 mg/kg body weight for 8 weeks. Isotype control antibody (IC) conjugated to DNA minor groove binder (PBD) at DAR 2 and chimeric 19E9 antibody conjugated to DNA minor groove binder (PBD) at DAR 2 were each administered at the dosage 0.05 mg/kg once per week for 8 weeks. Tumor volume was evaluated by measuring tumor diameters, with a calliper, biweekly during the whole experimental period. The formula TV (mm3)=[length (mm)×width (mm)2]/2 was used, where the length and the width are the longest and the shortest diameters of the tumor, respectively.

Soluble MICA was quantified using a sandwich Enzyme-Linked ImmunoSorbent Assay (ELISA). Appropriately-diluted unknown samples, calibrators and quality controls were distributed over a 96-well plate coated with a MICA-specific monoclonal antibody (clone AMO1; Novus Biologicals). After incubation, unbound substances were washed away and MICA captured by AMO1 was detected by the addition and incubation of a MICA/B specific biotinylated monoclonal antibody (clone BAMO3; Bamomab Inc.). The plate was then washed and streptavidin coupled to horseradish peroxidase (HRP) was added to all wells. After a final incubation and wash, the assay was revealed through the addition of a colorimetric reagent (3,3′,5,5′-Tetramethylbenzidine, TMB). The plate was read at 405 nm with a spectrophotometer. The developed color was proportional to the concentration of soluble MICA in the samples.

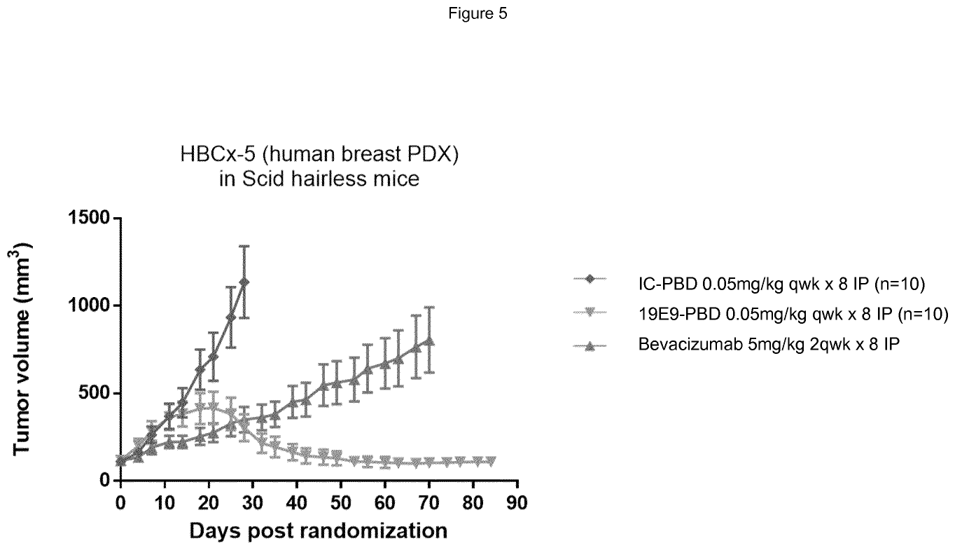

Results are shown in FIG. 5 (mean of 10 mice for each group). The anti-MICA-PBD immunoconjugate (19E9-PBD) showed strong anti-tumoral activity despite a dose of only 0.05 mg/kg once weekly, leading to substantially complete elimination of all tumors. The isotype control-PBD (IC-PBD) was not able to control tumor growth, while bevacizumab showed a partial slowing in growth of tumor volume, and this despite a dose 200 fold higher than that of 19E9-PBD.

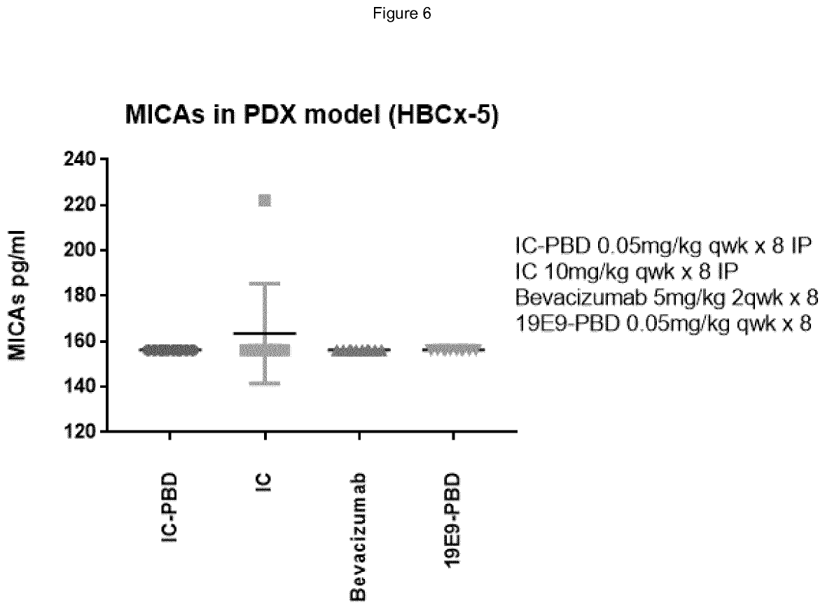

Soluble MICA (sMICA) concentration in blood from the PDX mice for each treatment was assessed in blood sampled just before mouse sacrifice. Interestingly, unlike what can be observed with treatment with anti-MICA antibody treatments in which an Fc-competent (e.g. human IgG1 that binds Fcγ receptors) is administered at a dose capable of mediating ADCC towards tumor cells (10 mg/kg), the anti-MICA-PBD immunoconjugates (19E9-PBD) administered at a dose of 0.05 mg/g did not cause an increase in soluble MICA in circulation (see FIG. 6).

### Example 7: Preparation of Fc-Mutated Antibodies

A parental antibody chADC2 binding to an antigen expressed at the surface of a variety of solid tumor types was modified by introduction of human VH and VL acceptor frameworks to yield humADC2, verified for retention of binding affinity to the target antigen, and was produced as a human IgG1 in a variety of different variants having different mutations in the heavy chain constant regions that each caused a reduction and/or loss of binding to human Fc receptors while retaining target antigen binding. The VH and Vk sequences of each antibody were cloned into vectors containing the huIgG1 CH1 constant domain and the huCk constant domain respectively. The two obtained vectors were co-transfected into the CHO cell line.

The amino acid sequence of the mutated Fc domains for each variant of the humADC2 antibody are shown below:

1. L234F/L235E/P331S mutation
(designated “humADC2-1”):
(SEQ ID NO: 105)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E [symbol] G G P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P A [symbol] I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K
2. L234A/L235E/P331S
mutation (designated “humADC2-2”):
(SEQ ID NO: 106)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E [symbol] G G P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P A [symbol] I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K
3. L234A/L235E/G237A/A330S/P331S mutation
(designated “humADC2-3”):
(SEQ ID NO: 107)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E [symbol] G [symbol] P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P [symbol] I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K
4. L234A/L235E/G237A/P331S mutation
(designated “humADC2-1”):
(SEQ ID NO: 108)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E [symbol] G [symbol] P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P A [symbol] I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K

### Example 8: Binding to FcγR

Antibodies having the Fc domains of shown in Example 7, as well are comparator antibodies, were evaluated to assess whether they could retain binding to Fcγ receptors.

SPR measurements were performed on a Biacore T100 apparatus (Biacore GE Healthcare) at 25° C. In all Biacore experiments HBS-EP+(Biacore GE Healthcare) and 10 mM NaOH, 500 mM NaCl served as running buffer and regeneration buffer respectively. Sensorgrams were analyzed with Biacore T100 Evaluation software. Recombinant human FcR's (CD64, CD32a, CD32b, CD16a and CD16b) were cloned, produced and purified.

Antibodies tested included: antibodies having wild type human IgG1 domain, antibodies having a human IgG4 domain with S241P substitution, human IgG1 antibodies having a N297S substitution, human IgG1 antibodies having L234F/L235E/P331S substitutions, human IgG1 antibodies having L234A/L235E/P331S substitutions, human IgG1 antibodies having L234A/L235E/G237A/A330S/P331S substitutions, and human IgG1 antibodies having L234A/L235E/G237A/P331S substitutions.

Antibodies were immobilized covalently to carboxyl groups in the dextran layer on a Sensor Chip CMS. The chip surface was activated with EDC/NHS (N-ethyl-N′-(3-dimethylaminopropyl) carbodiimidehydrochloride and N-hydroxysuccinimide (Biacore GE Healthcare)). Antibodies were diluted to 10 μg/ml in coupling buffer (10 mM acetate, pH 5.6) and injected until the appropriate immobilization level was reached (i.e. 800 to 900 RU). Deactivation of the remaining activated groups was performed using 100 mM ethanolamine pH 8 (Biacore GE Healthcare).

Monovalent affinity study was assessed following a classical kinetic wizard (as recommended by the manufacturer). Serial dilutions of soluble analytes (FcRs) ranging from 0.7 to 60 nM for CD64 and from 60 to 5000 nM for all the other FcRs were injected over the immobilized bispecific antibodies and allowed to dissociate for 10 min before regeneration. The entire sensorgram sets were fitted using the 1:1 kinetic binding model for CD64 and with the Steady State Affinity model for all the other FcRs.

The results are shown in Table 3, below. Results showed that while full length wild type human IgG1 bound to all human Fcγ receptors, and human IgG4 in particular bound significantly to CD64 (KD shown in the Table 3), the L234A/L235E/G237A/A330S/P331S substitutions and L234A/L235E/G237A/P331S substitutions abolished binding to CD64 as well as to CD16a.

### Example 9: BTG-Mediated Coupling onto Constant Regions of Fc-Mutated Antibodies

Antibodies humADC2-1, humADC2-2, humADC-3 and humADC2-4 produced in Example 7 were assessed for functionalization by bacterial transglutaminase. Acceptor glutamines within the Fc domain with a small lysine-based linker comprising a reactive group (an azide). The glutamine naturally present in the CH2 domain was chosen for evaluation (glutamine at Kabat residue 295 of each heavy chain).

5 mg/mL of each Fc-mutated antibody of Example 7 was deglycosylated with PNGase F overnight at 37° C. The deglycosylated mAb was then incubated with 20 equivalents of NH₂—PEG-N3 spacer per site of coupling (structure shown below) and 2 U/mL BTG overnight at 37° C. in PBS. The reaction was monitored by LC/MS (ESI-TOF).

NH2-PEG-N3 spacer:

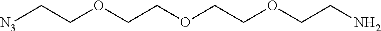 [SMILES: [N-]=[N+]=NCCOCCOCCOCCN]

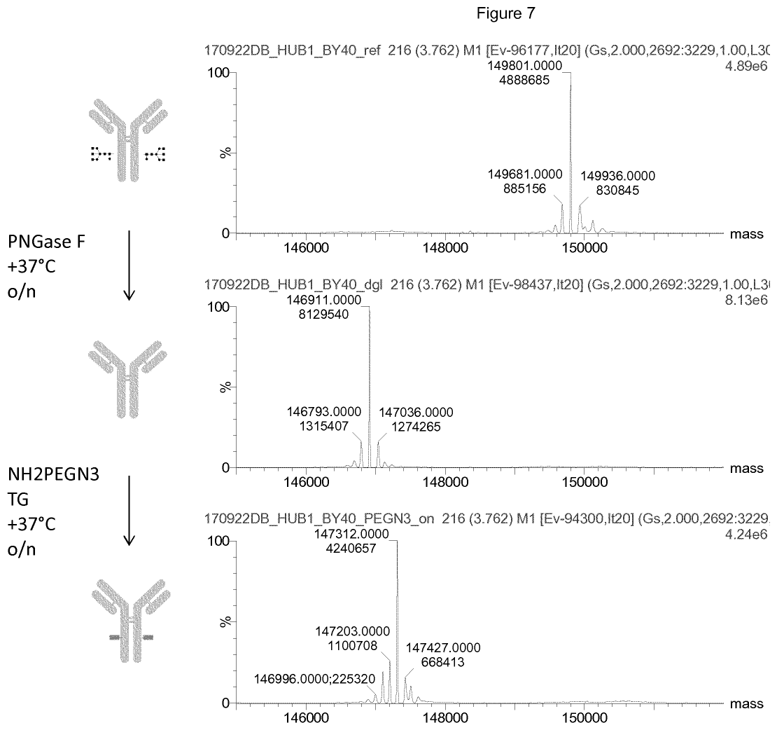

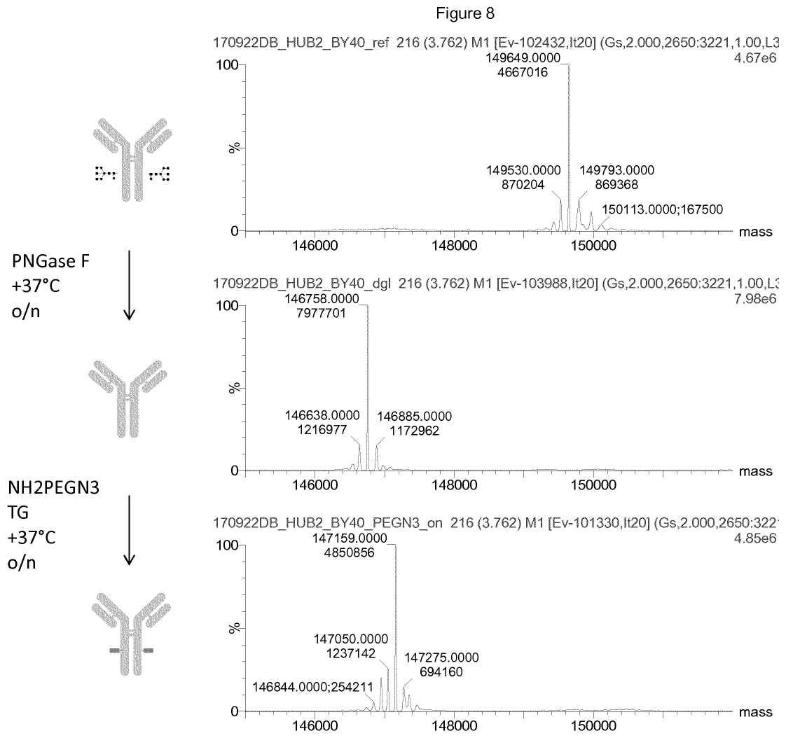

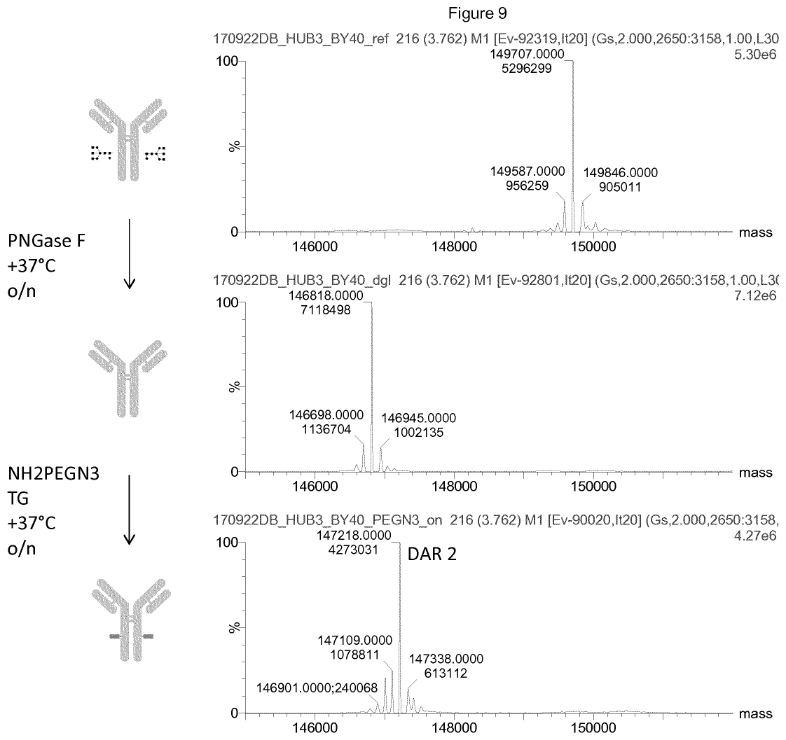

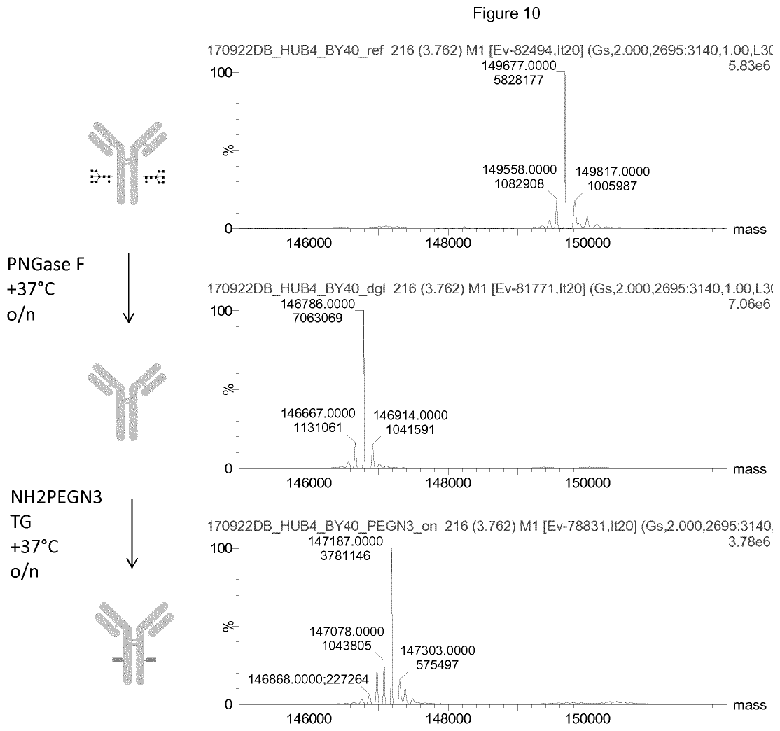

Results showed that BTG was able to couple the acceptor glutamine at residue 295 substantially completely, obtaining a drug:antibody ratio of 2.0 for each of antibodies humADC2-1, humADC2-2, humADC-3 and humADC2-4. Results are shown in FIGS. 7, 8, 9 and 10, respectively. FIG. 7 shows LC/MS analysis of the glycosylated starting antibody humADC2-1 (top panel), the deglycosylated antibody humADC2-1 (middle panel), and the antibody humADC2-1 coupled to NH₂—PEG-N3 (one NH₂—PEG-N3 on each acceptor glutamine per heavy chain). FIG. 8 shows LC/MS analysis of the glycosylated starting antibody humADC2-2 (top panel), the deglycosylated antibody humADC2-2 (middle panel), and the antibody humADC2-2 coupled to NH₂—PEG-N3 (one NH₂—PEG-N3 on each acceptor glutamine per heavy chain). FIG. 9 shows LC/MS analysis of the glycosylated starting antibody humADC2-3 (top panel), the deglycosylated antibody humADC2-3 (middle panel), and the antibody humADC2-3 coupled to NH₂—PEG-N3 (one NH₂—PEG-N3 on each acceptor glutamine per heavy chain). FIG. 10 shows LC/MS analysis of the glycosylated starting antibody humADC2-4 (top panel), the deglycosylated antibody humADC2-4 (middle panel), and the antibody humADC2-4 coupled to NH₂—PEG-N3 (one NH₂—PEG-N3 on each acceptor glutamine per heavy chain).

TABLE 3

| | | | | L234A/ | | | |
|---|---|---|---|---|---|---|---|
| | | | | L235E/ | L234A/ | Wild type | |
| | | L234F/ | L234A/ | G237A/ | L235E/ | human | Human IgG4 |
| | | L235E/ | L235E/ | A330S/ | G237A/ | IgG1 | antibody |
| Human Fc | N297S | P331S | P331S | P331S | P331S | antibody | with S241P |
| receptor | KD (nM) | KD (nM) | KD (nM) | KD (nM) | KD (nM) | KD (nM) | KD (nM) |
| CD64 | 278 | 933 | 1553 | No binding | No binding | 12.74 | 96.83 |
| CD32a | No binding | 14250 | 19900 | 18190 | 16790 | 2075 | 3218 |
| CD32b | No binding | 17410 | 79830 | 21800 | 16570 | 3914 | 2659 |
| CD16a(F) | No binding | 35580 | No binding | No binding | No binding | 961.9 | Low |
| | | | | | | | binding |
| CD16a(V) | No binding | 8627 | 9924 | No binding | No binding | 733.7 | 8511 |
| CD16b | No binding | No binding | No binding | No binding | No binding | 15020 | Low |
| | | | | | | | binding |
| FcRn | 712 | 627 | 1511 | 714 | 758 | 1272 | 1176 |

All headings and sub-headings are used herein for convenience only and should not be construed as limiting the invention in any way. Any combination of the above-described elements in all possible variations thereof is encompassed by the invention unless otherwise indicated herein or otherwise clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents, The description herein of any aspect or embodiment of the invention using terms such as reference to an element or elements is intended to provide support for a similar aspect or embodiment of the invention that “consists of′,” “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

All publications and patent applications cited in this specification are herein incorporated by reference in their entireties as if each individual publication or patent application were specifically and individually indicated to be incorporated by reference.

1-48. (canceled)

49. A method of treating an individual having a cancer characterized by MICA-expressing cells, comprising administering to the individual an antigen binding protein having the structure of formula I:
Ab-(X-Z)ₘ (formula I)
or a pharmaceutically acceptable salt or solvate thereof,
wherein,
Ab is an antibody or antibody fragment that binds a human MICA polypeptide, wherein the antibody or antibody fragment lacks an Fc domain or comprises an Fc domain of human origin that is modified to reduce binding to a human Fey receptor;
X is a moiety which connects Ab and Z;
Z comprises a DNA minor groove binding agent; and
m is 2.

50. The method of claim 49, wherein the antibody comprises an Fc domain of IgG1 isotype that lacks binding to human Fcγ receptors human CD16A, CD16B, CD32A, CD32B and CD64, wherein the Fc domain comprises an amino acid substitution at Kabat residue(s) 234, 235, 237, 297, 330 and/or 331.

51. The method of claim 50, wherein the Fe domain comprises L234A/L235E/N297X/P331S substitutions, L234F/L235E/N297X/P331S substitutions, L234A/L235E/G237A/N297X/P331S substitutions, or L234A/L235E/G237A/N297X/A330S/P331S substitutions, wherein X is any amino acid other than an asparagine.

52. The method of claim 49, wherein the antibody comprises a functionalized acceptor glutamine at Kabat residue 295 and/or 297 of each heavy chain.

53. The method of claim 49, wherein the cancer is a colorectal cancer, renal cell carcinoma, lung cancer, melanoma, ovarian cancer, endometrial cancer, pancreatic cancer or a head and neck cancer.

54. The method of claim 49, wherein the treatment is capable of causing the death of MICA-expressing tumor cells without inducing an increase in soluble MICA in circulation.

55. The method of claim 49, wherein the antigen binding protein is administered in an amount between 0.01 and 0.1 mg/kg body weight.

56. The method of claim 49, wherein the antigen binding protein is administered in an amount between 0.01 and 0.2 mg/kg body weight.

57. An antigen binding protein having the structure of formula I:
Ab-(X-Z)ₘ (formula I)
or a pharmaceutically acceptable salt or solvate thereof,
wherein,
Ab is an antibody or antibody fragment that binds a human MICA polypeptide, wherein the antibody or antibody fragment lacks an Fc domain or comprises an Fc domain of human origin that is modified to reduce binding to a human Fcγ receptor;
X is a moiety which connects Ab and Z;
Z comprises a DNA minor groove binding agent; and
m is 2.

58. The antigen binding protein of claim 57, wherein X comprises a linker that is cleaved by an intracellular peptidase or protease enzyme, optionally, a lysosomal or endosomal protease.

59. The antigen binding protein of claim 57, wherein the antibody or antibody fragment comprises a functionalized amino acid residue (B) comprising the structure:
(B)-L″-Y-Z
or a pharmaceutically acceptable salt or solvate thereof,
wherein:
B is an amino acid residue present within or appended to a constant region of the antibody or antibody fragment;
L″ is a linker covalently bonded to the amino acid residue B;
Y is a spacer system; and
Z comprises a DNA minor groove binding agent.

60. The antigen binding protein of claim 57, wherein the antibody or antibody fragment comprises a functionalized amino acid residue (B) comprising the structure comprising the structure:
(B)-L″-RR′—Y-Z
or a pharmaceutically acceptable salt or solvate thereof,
wherein:
B is an amino acid residue present within or appended to a constant region of the antibody or antibody fragment;
L″ is a linker covalently bonded to the amino acid residue B;
(RR′) is an addition product between a reactive moiety R and a complementary reactive moiety R′;
Y is a spacer system; and
Z comprises a DNA minor groove binding agent.

61. The antigen binding protein of claim 57, wherein Z is a pyrrolobenzodiazepine dimer.

62. The antigen binding protein of claim 57, wherein the antigen binding protein competes for binding to the same epitope on MICA and/or MICB as an antibody selected from the group consisting of: 19E9, 18E8, 9C10 or 6E4.

63. The antigen binding protein of claim 57, wherein the antigen binding protein is an antibody or antibody fragment comprising a functionalized acceptor glutamine residue (Q) comprising the structure:
(Q)-L″-Y-Z
or a pharmaceutically acceptable salt or solvate thereof,
wherein:
Q is a glutamine residue present within or appended to a constant region of the antibody or antibody fragment;
L″ is a lysine-based linker in which the nitrogen atom is covalently bonded to the γ carbon of Q as a secondary amine;
Y is a spacer system; and
Z comprises a cytotoxic agent, optionally a DNA minor groove binding agent, optionally a pyrrolobenzodiazepine.

64. The antigen binding protein of claim 57, wherein the antibody comprises an Fc domain of IgG1 isotype that has decreased binding to human Fcγ receptors human CD16A, CD16B, CD32A, CD32B and CD64, wherein the Fc domain comprises an amino acid substitution at Kabat residue(s) 234, 235, 237, 297, 330 and/or 331.

65. The antigen binding protein of claim 64, wherein the Fc domain comprises L234A/L235E/N297X/P331S substitutions, L234F/L235E/N297X/P331S substitutions, L234A/L235E/G237A/N297X/P331S substitutions, or L234A/L235E/G237A/N297X/A330S/P331S substitutions, wherein X is any amino acid other than an asparagine.

66. A composition comprising a plurality of immunoconjugates according to claim 57, wherein the composition is characterized by a ratio of cytotoxic agent moieties to antibody molecules (drug:antibody ratio) of 1.8 to 2.0.


SEQUENCE LISTING (108 sequences)

SEQ ID NO: 1 - protein, 274 aa, Homo sapiens
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 2 - protein, 274 aa, Homo sapiens
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNVETEEWTVPQSSRAQTLAMNVRNFLKEDAMKTKTHYHAMHADCLQELRRYLESSVVLRRRVPPMVNVTRSEASEGNITVTCRASSFYPRNITLTWRQDGVSLSHDTQQWGDVLPDGNGTYQTWVATRICQGEEQRFTCYMEHSGNHSTHPVPS

SEQ ID NO: 3 - protein, 274 aa, Homo sapiens
EPHSLRYNLTVLSWDGSVQSGFLAEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETEEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 4 - protein, 274 aa, Homo sapiens
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNLETEEWTVPQSSRAQTLAMNVRNFLKEDAMKTKTHYHAMHADCLQELRRYLESGVVLRRTVPPMVNVTRSEASEGNITVTCRASSFYPRNIILTWRQDGVSLSHDTQQWGDVLPDGNGTYQTWVATRICRGEEQRFTCYMEHSGNHSTHPVPS

SEQ ID NO: 5 - protein, 274 aa, Homo sapiens
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNLETEEWTVPQSSRAQTLAMNVRNFLKEDAMKTKTHYHAMHADCLQELRRYLESSVVLRRTVPPMVNVTRSEASEGNITVTCRASSFYPRNIILTWRQDGVSLSHDTQQWGDVLPDGNGTYQTWVATRICRGEEQRFTCYMEHSGNHSTHPVPS

SEQ ID NO: 6 - protein, 383 aa, Homo sapiens [sequence omitted]

SEQ ID NO: 7 - protein, 118 aa, Mus musculus
EVQLVESGGALVKPGGSLKLSCAASGFTFSYYAMSWVRQTPEKRLEWVATISRGGNYIYYTDSVKGRFTISRDNAKNTLYLQMTSLRSEDTAMFYCASISDYDGAWLAYWGQGTLVTV

SEQ ID NO: 8 - protein, 112 aa, Mus musculus
DVLMTQTPLSLPVSLGDQASISCRSSQSIIHTNGNTYLEWYLQKPGQSPKLLIYKISNRFSGVPDRFSGSGSGTDFTLKISRVEAEDLGVYYCFQGSHVPWTFGGGTKLEIK

SEQ ID NO: 9 - protein, 118 aa, Mus musculus
QITLKESGPGILKPSQTLSLTCSFSGFSLSTSGMGVGWIRQPSGKGLEWLAHIWWDDDKYYNPSLKSQLTISKDTSRNQVFLRITSVDTADTATYYCARRTQGYFDYWGQGTTLTVSS

SEQ ID NO: 10 - protein, 107 aa, Mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPWTFGGGTKLEIK

SEQ ID NO: 11 - protein, 118 aa, Mus musculus
EVQLVESGGGLVKPGGSLKLSCAASGFTFSRYAMSWVRQTPEKRLEWVATIFSGGSYTYYPDSVKGRFTISRDNANNTLYLQMSSLKAEDTAMYFCARPNWERTFDYWGQGTTLTVSS

SEQ ID NO: 12 - protein, 113 aa, Mus musculus
DIVMTQSPSSLAMSVGQKVTMSCKSSQSLLNSSNQKNYLAWYQQKPGQSPKLLVYFASTRESGVPDRFMGSGSGTDFTLTISSVQAEDLADYFCQQHYSTPPTFGGGTKLEIK

SEQ ID NO: 13 - protein, 133 aa, Mus musculus
MRVLILLWLFTAFPGLLSDVQLQESGPGLVKPSQSLSLTCTVTGYSITSDYAWNWIRQFPGNKLEWMGFVSYSGTTKYNPSLKSRISITRDTSENQFFLQLNSVTSEDTATYYCARGYGFDYWGQGTTLTVSS

SEQ ID NO: 14 - protein, 108 aa, Mus musculus
EIVLTQSPTTMAASPGEKITITCSATSSISSIYFHWYQQRPGFSPKLLIYRTSNLASGVPARFSGSGSGTSYSLTIGTMEAEDVATYYCQQGTTIPFTFGSGTKLEIK

SEQ ID NO: 15 - protein, 115 aa, artificial
  Chimeric
QVQLQESGPGLVKPSETLSLTCTVSGYSITSDYAWNWIRQPPGKGLEWIGFVSYSGTTKYNPSLKSRVTISRDTSKNQFSLKLSSVTAADTAVYYCARGYGFDYWGQGTTVTVSS

SEQ ID NO: 16 - protein, 108 aa, Artificial
  Chimeric
EIVLTQSPATLSLSPGERATLSCSATSSISSIYFHWYQQKPGQAPRLLIYRTSNLASGIPARFSGSGSGTDYTLTISSLEPEDFAVYYCQQGTTIPFTFGQGTKLEIK

SEQ ID NO: 17 - protein, 115 aa, Artificial; identical to SEQ ID NO: 15
  Chimeric

SEQ ID NO: 18 - protein, 108 aa, Artificial
  Chimeric
EIVLTQSPATLSLSPGERATLSCSATSSISSIYFHWYQQKPGQAPRLLIYRTSNLASGIPARFSGSGSGTSYTLTISSLEPEDFAVYYCQQGTTIPFTFGQGTKLEIK

SEQ ID NO: 19 - protein, 115 aa, Artificial; identical to SEQ ID NO: 15
  Chimeric

SEQ ID NO: 20 - protein, 108 aa, Artificial
  Chimeric
EIVLTQSPATLSLSPGERATLSCSATSSISSIYFHWYQQKPGQAPRLLIYRTSNLASGIPARFSGSGSGTDYTLTISSLEPEDVAVYYCQQGTTIPFTFGQGTKLEIK

SEQ ID NO: 21 - protein, 118 aa, Mus musculus
QVQLQQPGAELVRPGTSVNLSCKASGYSFTRYWMNWVKQRPGQGLEWIGMIHPSDSETRLNQKFKDKATLTVDKSSSTAYMQLSSPTSEDSAVYYCGYGNFFYVMDYWGQGTSVTVSS

SEQ ID NO: 22 - protein, 107 aa, Mus musculus
DILLTQSPAILSVSPGERVSFSCRASQSIGTSIHWYQQRTNGSPRLLIKFASESISGIPSRFSGSGSGTDFTLNINSVESEDIADYYCQQSNFWPFTFGSGTKLEVK

SEQ ID NO: 23 - protein, 121 aa, Mus musculus
QVQLQQSGADLVRPGASVRLSCRASGYSFTNYWMNWVKQRPGQGLEWIGMIHPSDSETRLNQKFKDKATLTVDKSSNTAYMQLSSPTSEDSAIYYCARDDFFTMDYWGQGTSVTVSSASTK

SEQ ID NO: 24 - protein, 107 aa, Mus musculus
DILLTQSPAILSVSPGERVSFSCRASQNIVTSIHWYQQSTNGSPRLLIKYASESISGIPSRFSGSGSGTDFTLTINSVESEDVADYYCQQSNIWPLTFGAGTKLELK

SEQ ID NO: 25 - protein, 122 aa, Mus musculus
QVTLKESGPGILKPSQTLSLTCSFSGFSLSTSGMGVGWIRQPSGKGLEWLAHIWWDDDRYYNPSLKSQLTISKDTSRNQVFLKITSVDTADTATYYCARRLNGYFDYWGQGTTLTVSSASTK

SEQ ID NO: 26 - protein, 107 aa, Mus musculus
DIVMTQSPATLSVTLGDRVSLSCRASQSISDYLHWYQQKSHESPRLLIKYASQSISGIPSRFSGSGSGSDFTLSINSVEPEDVGVYYCQNGHSFPFTFGSGTKLEIK

SEQ ID NO: 27 - protein, 123 aa, Mus musculus
DVQLQESGPDLVNPSQSLSLICTVTGYSITSDYSWHWIRQFPGNKLEWMGNIHYSGRINYNPSLRSRISITRDTSKNQFFLQLISVTTEDTATYYCATRRTFGNFEDYWGQGTTLTVSSASTK

SEQ ID NO: 28 - protein, 106 aa, Mus musculus
QIVLSQSPATLSVSPGEKVTMTCRSSSSVNYMHWYQQKPGSSPKPWIYATSTLASGVPARFSGSGSGTSYSLTISRVEAEDAATYYCQQWSSNPLTFGAGTKLELK

SEQ ID NO: 29 - protein, 143 aa, Mus musculus
MEWSWVFLFFLSVTTGVHSQVQLQQSAAELARPGASVKMSCKASGYTFTSYTMHWVKQRPGQGLEWIGYINPSSGYTEYNQKFKDKTTLTVDKSSTTSYMQLSSLTSDNSAVYYCARGGDWDVDWFVYWGQGTLVTVSAASTK

SEQ ID NO: 30 - protein, 106 aa, Mus musculus
QIVLTQSPAIMSASPGEKVTITCSASSSISYMHWFQQKPGTSPKLWIYSTSKLASGVPARFSGSGSGTSHSLTISRMEAEDAATYYCQHRSTYPFTFGSGTKLEIK

SEQ ID NO: 31 - protein, 122 aa, Mus musculus
DVQLQESGPDLVKPSQSLSLTCTVTGYSITSGYSWHWIRQFPGNKLEWMGFIHYSGSTDYNPSLKSRISLTRDTSKNQFFLQLNSVSTEDTATYYCAKDYGHWYFDVWGAGTTVTVSSASTK

SEQ ID NO: 32 - protein, 122 aa, Mus musculus; identical to SEQ ID NO: 31

SEQ ID NO: 33 - protein, 122 aa, Mus musculus
QVQLQQPGAELVRPGASVKLSCKASGYSFTSYWMNWMKQRPGQGLEWIGMIHPSDSETRLNQKFKDKATLTVDKSSSTAYMQLNSPTSEDSAVYYCAREMGPYTLDYWGQGTSVTVSSASTK

SEQ ID NO: 34 - protein, 107 aa, Mus musculus
DILLTQSPAILSVSPGARVSFSCRASQNIDTSIHWYQQRTNGSPRLLIKYASESISGIPSRFSGSGSGTDFTLSINSVESEDIADYYCQQSNYWPLTFGAGTKLELK

SEQ ID NO: 35 - protein, 6 aa, Mus musculus
SYYAMS

SEQ ID NO: 36 - protein, 17 aa, Mus musculus
TISRGGNYIYYTDSVKG

SEQ ID NO: 37 - protein, 11 aa, Mus musculus
ISDYDGAWLAY

SEQ ID NO: 38 - protein, 16 aa, Mus musculus
RSSQSIIHTNGNTYLE

SEQ ID NO: 39 - protein, 7 aa, Mus musculus
KISNRFS

SEQ ID NO: 40 - protein, 9 aa, Mus musculus
FQGSHVPWT

SEQ ID NO: 41 - protein, 7 aa, Mus musculus
TSGMGVG

SEQ ID NO: 42 - protein, 15 aa, Mus musculus
HIWWDDDKYYNPSLK

SEQ ID NO: 43 - protein, 15 aa, Mus musculus
HIWWDDDKYYNPSLK

SEQ ID NO: 44 - protein, 11 aa, Mus musculus
RASQSISDYLH

SEQ ID NO: 45 - protein, 7 aa, Mus musculus
YASQSIS

SEQ ID NO: 46 - protein, 9 aa, Mus musculus
QNGHSFPWT

SEQ ID NO: 47 - protein, 5 aa, Mus musculus
RYAMS

SEQ ID NO: 48 - protein, 15 aa, Mus musculus
TIFSGGSYTYYPDSV

SEQ ID NO: 49 - protein, 9 aa, Mus musculus
PNWERTFDY

SEQ ID NO: 50 - protein, 16 aa, Mus musculus
KSSQSLLNSSNQKNYL

SEQ ID NO: 51 - protein, 7 aa, Mus musculus
FASTRES

SEQ ID NO: 52 - protein, 9 aa, Mus musculus
QQHYSTPPT

SEQ ID NO: 53 - protein, 6 aa, Mus musculus
SDYAWN

SEQ ID NO: 54 - protein, 16 aa, Mus musculus
FVSYSGTTKYNPSLKS

SEQ ID NO: 55 - protein, 6 aa, Mus musculus
GYGFDY

SEQ ID NO: 56 - protein, 12 aa, Mus musculus
SATSSISSIYFH

SEQ ID NO: 57 - protein, 7 aa, Mus musculus
RTSNLAS

SEQ ID NO: 58 - protein, 9 aa, Mus musculus
QQGTTIPFT

SEQ ID NO: 59 - protein, 5 aa, Mus musculus
RYWMN

SEQ ID NO: 60 - protein, 17 aa, Mus musculus
MIHPSDSETRLNQKFKD

SEQ ID NO: 61 - protein, 9 aa, Mus musculus
GNFFYVMDY

SEQ ID NO: 62 - protein, 11 aa, Mus musculus
RASQSIGTSIH

SEQ ID NO: 63 - protein, 7 aa, Mus musculus
ASESISG

SEQ ID NO: 64 - protein, 9 aa, Mus musculus
QQSNFWPFT

SEQ ID NO: 65 - protein, 5 aa, Mus musculus
NYWMN

SEQ ID NO: 66 - protein, 17 aa, Mus musculus
MIHPSDSETRLNQKFKD

SEQ ID NO: 67 - protein, 8 aa, Mus musculus
DDFFTMDY

SEQ ID NO: 68 - protein, 11 aa, Mus musculus
RASQNIVTSIH

SEQ ID NO: 69 - protein, 7 aa, Mus musculus
YASESIS

SEQ ID NO: 70 - protein, 9 aa, Mus musculus
QQSNIWPLT

SEQ ID NO: 71 - protein, 7 aa, Mus musculus
TSGMGVG

SEQ ID NO: 72 - protein, 16 aa, Mus musculus
HIWWDDDRYYNPSLKS

SEQ ID NO: 73 - protein, 8 aa, Mus musculus
RLNGYFDY

SEQ ID NO: 74 - protein, 11 aa, Mus musculus
RASQSISDYLH

SEQ ID NO: 75 - protein, 7 aa, Mus musculus
YASQSIS

SEQ ID NO: 76 - protein, 9 aa, Mus musculus
QNGHSFPFT

SEQ ID NO: 77 - protein, 6 aa, Mus musculus
SDYSWH

SEQ ID NO: 78 - protein, 16 aa, Mus musculus
NIHYSGRINYNPSLRS

SEQ ID NO: 79 - protein, 10 aa, Mus musculus
RRTFGNFEDY

SEQ ID NO: 80 - protein, 10 aa, Mus musculus
RSSSSVNYMH

SEQ ID NO: 81 - protein, 7 aa, Mus musculus
ATSTLAS

SEQ ID NO: 82 - protein, 9 aa, Mus musculus
QQWSSNPLT

SEQ ID NO: 83 - protein, 5 aa, Mus musculus
SYTMH

SEQ ID NO: 84 - protein, 17 aa, Mus musculus
YINPSSGYTEYNQKFKD

SEQ ID NO: 85 - protein, 11 aa, Mus musculus
GGDWDVDWFVY

SEQ ID NO: 86 - protein, 10 aa, Mus musculus
SASSSISYMH

SEQ ID NO: 87 - protein, 7 aa, Mus musculus
STSKLAS

SEQ ID NO: 88 - protein, 9 aa, Mus musculus
QHRSTYPFT

SEQ ID NO: 89 - protein, 6 aa, Mus musculus
SGYSWH

SEQ ID NO: 90 - protein, 16 aa, Mus musculus
FIHYSGSTDYNPSLKS

SEQ ID NO: 91 - protein, 9 aa, Mus musculus
DYGHWYFDV

SEQ ID NO: 92 - protein, 11 aa, Mus musculus
KASQSVSYDVA

SEQ ID NO: 93 - protein, 7 aa, Mus musculus
YASNRYT

SEQ ID NO: 94 - protein, 8 aa, Mus musculus
QQDYSSLT

SEQ ID NO: 95 - protein, 5 aa, Mus musculus
SYWMN

SEQ ID NO: 96 - protein, 17 aa, Mus musculus
MIHPSDSETRLNQKFKD

SEQ ID NO: 97 - protein, 9 aa, Mus musculus
EMGPYTLDY

SEQ ID NO: 98 - protein, 11 aa, Mus musculus
RASQNIDTSIH

SEQ ID NO: 99 - protein, 7 aa, Mus musculus
YASESIS

SEQ ID NO: 100 - protein, 9 aa, Mus musculus
QQSNYWPLT

SEQ ID NO: 101 - protein, 330 aa, Homo sapiens
  X (180)..(180): X may be any amino acid
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 330 aa, Homo sapiens
  X (180)..(180): X may be any amino acid
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 330 aa, Homo sapiens
  X (180)..(180): X may be any amino acid
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYXSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 330 aa, Homo sapiens
  misc_feature (180)..(180): Xaa can be any naturally occurring amino acid
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYXSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 330 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK